(19) United States
(12) Patent Application Publication
(10) Pub. No.: US 2026/0327286 A1
(43) Pub. Date: Oct. 22, 2026

(54) ENRICHMENT OF CLINICALLY RELEVANT CELL TYPES USING RECEPTORS

(71) Applicant: The Board of Trustees of the Leland Stanford Junior University, Stanford, CA (US)

(72) Inventors: Michael Kyle Cromer, Stanford, CA (US); Matthew H. Porteus, Stanford, CA (US); Carsten Charlesworth, Stanford, CA (US)

(73) Assignee: The Board of Trustees of the Leland Stanford Junior University, Stanford, CA (US)

(21) Appl. No.: 19/119,379

(22) PCT Filed: Oct. 16, 2023
(86) PCT No.: PCT/US2023/076969

§ 371 Date: Apr. 8, 2025

Related U.S. Application Data
(60) Provisional application No. 63/416,906, filed on Oct. 17, 2022.

(51) Int. Cl. C07K 14/705; A61K 35/12; A61K 35/28; A61K 45/06; A61K 48/00; C12N 5/0789; C12N 9/22; C12N 15/10; C12N 15/113; C12N 15/864

(52) U.S. Cl. CPC C07K 14/705; A61K 35/28; A61K 45/06; A61K 48/00; C12N 5/0647; C12N 9/226; C12N 15/102; C12N 15/113; C12N 15/8645; A61K 2035/124; C07K 2319/03; C07K 2319/70; C12N 2310/20; C12N 2310/315; C12N 2310/321; C12N 2506/11; C12N 2510/00; C12N 2750/14143; C12N 2830/34

(57) ABSTRACT

The present disclosure provides methods and compositions for tunable differentiation of hematopoietic stem and progenitor cells (HSPCs) in response to small molecules by using genetically modified HSPCs that express chimeric transmembrane receptors.

### CROSS-REFERENCE TO RELATED APPLICATIONS

This application claims priority to U.S. Provisional Application No. 63/416,906, filed Oct. 17, 2022, the disclosure of which is herein incorporated by reference in its entirety for all purposes.

### BACKGROUND

Although it is possible to cure hemoglobinopathies such as sickle cell anemia and beta-thalassemia using allogeneic hematopoietic stem cell transplantation (allo-HSCT), less than 20% of patients have an available immunologically matched donor without the disease. Therefore, a number of gene therapies and genome editing strategies have been developed to address this need, by correcting a patient's own HSCs ex vivo and then transplanting the cells back into the patient via autologous-HSCT (auto-HSCT). Using a patient's own cells thereby minimizes the risk of immune rejection or graft-versus-host disease (GvHD). However, in spite of the tremendous cost and dramatically reduced expected lifespan associated with sickle cell disease and beta-thalassemia, potentially curative allo- and auto-HSCT is only performed rarely, in the most severe instances of disease. This is because current HSCT protocols require a devastating myeloablation regimen to clear out the bone marrow (BM) niche to make room for transplanted HSCs to engraft. These radio- or chemo-therapeutic conditioning regimens carry a high risk of short-term and long-term effects, such as prolonged immunosuppression resulting from non-specific clearance of a patient's immune cells as well as the possibility of malignancy caused by the genotoxic stress placed on the BM.

The general requirement for clearance of the BM niche stands as a huge barrier to efficacy in the clinic. All current and future protocols require an intensive myeloablation step to achieve high frequencies of healthy donor or genome-edited HSC chimerism in the BM. The current threshold needed to achieve a functional cure (i.e., transfusion-independence) is ~25% corrected HSC chimerism, which then is expected to yield ~25% corrected red blood cell (RBC) chimerism in the bloodstream. However, this assumes that HSC chimerism in the BM is roughly equivalent to RBC chimerism in the bloodstream (in the absence of any positive selection for corrected cells, which occurs to a very minimal degree in the hemoglobinopathies).

While edited HSCs yield genome-corrected cells of all lineages (T cells, B cells, macrophages, etc.), the only cell type of clinical relevance to the hemoglobinopathies is the RBC. Therefore, technology to bias edited HSCs toward the erythroid lineage could allow low HSC chimerism in the BM to yield high levels of RBC chimerism in the bloodstream.

Truncations in the erythropoietin receptor (EPOR) have been shown to cause benign erythrocytosis-yielding non-pathogenic hyper-production of RBCs. One mutation, which often presents as a heterozygous nonsense mutation, truncates a portion of the C-terminus of EPOR thereby removing a negative inhibitory binding domain for SHP1. Further studies have shown that this does not create a constitutively active EPOR signaling cascade, but rather imparts hyper-sensitivity to the EPO cytokine.

Introduction of truncated EPOR to HSCs, either by disrupting the endogenous locus or driving expression of a truncated EPOR (tEPOR) cDNA, can impart a dramatic erythroid bias to edited HSCs. These EPOR truncations were also effectively paired with genome editing-based beta-thalassemia correction strategy in order to enrich for RBCs harboring a corrective edit. However, this system relies on activation of the truncated receptor by endogenous EPO cytokine levels, with little ability to titrate the effect post-transplantation. A concern with this strategy is that overexpression of rEPOR cDNA could eventually lead to pathogenic expansion of the erythroid compartment in a clinical presentation akin to polycythemia vera. Therefore, an ideal solution would place activation of the EPOR signaling cascade under control of a clinically relevant small molecule, to allow inducible erythroid bias to be initiated post-transplantation.

There is therefore a need for new compositions and methods for selectively driving the differentiation of stem cells, such as the differentiation of hematopoietic stem cells towards red blood cells. The present disclosure satisfies this need and provides other advantages as well.

### BRIEF SUMMARY

In one aspect, the present disclosure provides a chimeric transmembrane receptor polypeptide comprising: an extramembrane dimerizer domain, wherein the extramembrane dimerizer domain induces dimerization of the chimeric transmembrane receptor polypeptide upon recognition of a dimerization signal: a transmembrane domain; and an intramembrane domain, wherein the intramembrane domain is configured to induce activation of one or more intramembrane signal pathways upon dimerization of the chimeric transmembrane receptor polypeptide in a modified primary human cell comprising the chimeric transmembrane receptor polypeptide, and wherein the one or more intramembrane signaling pathways promote survival, proliferation, and/or differentiation of the modified primary human cell.

In some embodiments, the dimerization signal is a pharmaceutically acceptable small molecule dimerization signal.

In some embodiments, the extramembrane dimerizer domain comprises an FKBP domain, an mFRB domain, an HSV-TK dimerization domain, a rapamycin-inducible dimerization domain, a rapalogue-inducible dimerization domain, or a combination thereof. In some embodiments, the extramembrane dimerizer domain comprises an FKBP domain and the small molecule dimerization signal comprises AP20187 (BB dimerizer).

In some embodiments, the intramembrane domain comprises an EPOR intracellular domain, an SCF or CDI 17 intracellular domain, a TPOR or MPL intracellular domain, an EGFR intracellular domain, an RET intracellular domain, a CSF1R intracellular domain, an IGF1R intracellular domain, or a combination thereof. In some embodiments, the intramembrane domain comprises an EPOR intracellular domain and wherein dimerization of the chimeric transmembrane receptor polypeptide induces activation of intramembrane signal pathways resulting in increased survival, increased proliferation, and/or increased erythroid differentiation of the modified primary human cell upon recognition of the dimerization signal. In some embodiments, the intramembrane domain comprises a TPOR intracellular domain and wherein dimerization of the chimeric transmembrane receptor polypeptide induces activation of intramembrane signal pathways resulting in increased survival, increased proliferation, and/or increased erythroid differentiation of the modified primary human cell upon recognition of the dimerization signal. In some embodiments, the intramembrane domain comprises an SCF intracellular domain and wherein dimerization of the chimeric transmembrane receptor polypeptide induces activation of intramembrane signal pathways resulting in increased survival, increased proliferation, and/or increased erythroid differentiation of the modified primary human cell upon recognition of the dimerization signal.

In some embodiments, the extramembrane dimerizer domain is immediately adjacent to the transmembrane domain. In some embodiments, the extracellular domain does not bind erythropoietin (EPO).

In some embodiments, the chimeric transmembrane receptor polypeptide further comprises a signal peptide. In some embodiments, the signal peptide promotes membrane localization of the chimeric transmembrane receptor polypeptide. In some embodiments, the signal peptide comprises an IL6 signal peptide, an EPOR signal peptide, a lysozyme C signal peptide, an angiotensinogen signal peptide, an RNASE1 signal peptide, an RNASE3 signal peptide, or a modified human albumin signal peptide.

In some embodiments, the chimeric transmembrane receptor polypeptide further comprises a linker peptide. In some embodiments, the linker peptide comprises the amino acid sequence GGGGS.

In some embodiments, the chimeric transmembrane receptor polypeptide comprises an amino acid sequence having at least 80% identity to at least one of SEQ ID NOS: 1 to 3 or 7 to 11.

In another aspect, the present disclosure provides a recombinant nucleic acid encoding any of the chimeric transmembrane receptor polypeptides described herein.

In another aspect, the present disclosure provides a DNA construct comprising a promoter operably linked to the recombinant nucleic acid. In some embodiments, the promoter is an endogenous EPOR promoter, an endogenous HBA1 promoter, an endogenous TPOR promoter, a constitutive SFFV promoter, a constitutive PGK promoter, or a constitutive UbC promoter.

Also provided is a vector comprising any of the recombinant nucleic acids described herein or any of the DNA constructs described herein.

In another aspect, the present disclosure provides a host cell comprising any of the recombinant nucleic acids described herein, any of the DNA constructs described herein, or any of the vectors described herein. In some embodiments, the recombinant nucleic acid, DNA construct, or vector is integrated into the EPOR locus, the CCR5 locus, or the HBA1 locus. In some embodiments, the host cell is a eukaryotic cell. In some embodiments, the host cell is a primary human cell. In some embodiments, the host cell is a hematopoietic stem cell, a hematopoietic progenitor cell, a T cell, a B cell, an airway basal stem cell, or a pluripotent stem cell (PSC). In some embodiments, the host cell was derived from a patient who is a carrier of an allele that causes a genetic disorder. In some embodiments, the genetic disorder is beta-thalassemia, sickle cell disease (SCD), severe combined immunodeficiency (SCID), mucopolysaccharidosis type 1, Cystic Fibrosis, Gaucher disease, Krabbe disease, X-linked chronic granulomatous disease (X-CGD), or a combination thereof. In some embodiments, the genetic disorder is a hemoglobinopathy. In some instances, the hemoglobinopathy is beta-thalassemia or sickle cell disease (SCD). In some embodiments, the genome of the host cell is edited to contain a corrected version of the allele that causes the genetic disorder.

In another aspect, the present disclosure provides a method of inducing erythroid differentiation of a hematopoietic stem and progenitor cell (HSPC), the method comprising contacting an HSPC that comprises a recombinant nucleic acid encoding a chimeric transmembrane receptor polypeptide with the dimerization signal.

In another aspect, the present disclosure provides a method of tuning red blood cell levels in a patient, the method comprising: (i) editing an HSPC so that it can express at least one of the chimeric transmembrane receptor polypeptides described herein; (ii) transferring the resulting HSPC to the patient; (iii) administering a first quantity of the dimerization signal to the patient; (iv) monitoring red blood cell levels in the patient; and (v) administering a second quantity of the dimerization signal to the patient that is the same, less, or more than the first quantity of dimerization signal.

In another aspect, the present disclosure provides a method of increasing the proportion of red blood cells with an altered version of an allele associated with a genetic disorder, the method comprising: (i) creating an edited HSPC by introducing an altered version of the allele associated with a genetic disorder and a polynucleotide that encodes at least one of the chimeric transmembrane receptor polypeptides described herein into an HSPC; (ii) transferring the edited HSPC to the patient; and (iii) administering the dimerization signal to the patient. In some embodiments, the HSPC is derived from the patient's own cells. In some embodiments, the HSPC is derived from an allogeneic donor's cells. In some embodiments, the genetic disorder is beta-thalassemia, sickle cell disease (SCD), severe combined immunodeficiency (SCID), mucopolysaccharidosis type 1, Cystic Fibrosis. Gaucher disease. Krabbe disease, X-linked chronic granulomatous disease (X-CGD), or a combination thereof.

In another aspect, the present disclosure provides a method of genetically modifying a primary human cell, the method comprising introducing into the cell a chimeric transmembrane receptor polypeptide described herein. In some embodiments, the method comprises: (i) introducing into the cell a site-directed nuclease (SDN) targeted to a cleavage site at a genetic locus of interest; and (ii) introducing a homologous repair template into the cell, wherein the homologous repair template comprises a nucleotide sequence that is homologous to the locus of interest, wherein the site-directed nuclease cleaves the locus at the cleavage site, and the homologous repair template is integrated at the site of the cleaved locus by homology directed repair (HDR). In some embodiments, the SDN is an RNA-guided nuclease and the method further comprises introducing into the cell a single guide RNA (sgRNA) targeting the cleavage site, wherein the sgRNA directs the RNA-guided nuclease to the cleavage site. In some embodiments, the sgRNA comprises 2′-O-methyl-3′-phosphorothioate (MS) modifications at one or more nucleotides. In some embodiments, the MS modifications are present at the terminal nucleotides of the 5′ and 3′ ends. In some embodiments, the RNA-guided nuclease is Cas9. In some embodiments, the sgRNA and RNA-guided nuclease are introduced into the cell as a ribonucleoprotein (RNP). In some embodiments, the RNP is introduced into the cell by electroporation. In some embodiments, the homologous repair template is introduced into the cell using an adeno-associated virus serotype 6 (AAV6) vector. In some embodiments, the primary human cell is a hematopoictic stem cell, a hematopoietic progenitor cell, a T cell, a B cell, an airway basal stem cell, or a pluripotent stem cell (PSC). In some embodiments, the locus of interest is a gene selected from the group consisting of Erythropoietin Receptor (EPOR), Hemoglobin Subunit Beta (HBB), C-C Motif Chemokine Receptor 5 (CCR5), Interleukin 2 Receptor Subunit Gamma (IL2RG), Hemoglobin Subunit Alpha 1 (HBA1), Stimulator Of Interferon Response cGAMP Interactor 1 (STING1) and Cystic Fibrosis Transmembrane Conductance Regulator (CFTR).

In another aspect, the present disclosure provides a method of treating a genetic disorder in a human subject in need thereof, the method comprising: (i) providing an isolated primary cell from the subject: (ii) genetically modifying the primary cell using the method described herein, wherein the integration of the homologous donor template at the locus of interest in the cell alters an allele at the locus that is associated with the genetic disorder or leads to the expression of a therapeutic protein in the cell that is absent or deficient in the subject; and (iii) reintroducing the genetically modified cell into the subject. In some embodiments, the genetic disorder is beta-thalassemia, sickle cell disease (SCD), severe combined immunodeficiency (SCID), mucopolysaccharidosis type 1, Cystic Fibrosis, Gaucher disease, Krabbe disease, X-linked chronic granulomatous disease (X-CGD), or a combination thereof.

In another aspect, the present disclosure provides a method of generating a population of red blood cell n vitro, the method comprising: (i) editing one or more HSPCs to express at least one of the chimeric transmembrane receptor polypeptides as disclosed herein; and (ii) contacting the one or more HSPCs with a dimerization signal. In some embodiments, the dimerization signal is a small molecule dimerization signal. In some embodiments, the small molecule dimerization signal is AP20187 (BB dimerizer). In some embodiments, step (ii) further comprises contacting the one or more HSPCs with erythropoietin (EPO). In some embodiments, the method is conducted in a bioreactor.

### BRIEF DESCRIPTION OF THE DRAWINGS

FIG. 1: Diagram of novel AAV integration vectors that place coding regions from FKBP (taken from Martin et al. Nature Communications, 2020) and EPOR in various orientations. Each of the initial constructs targeted the CCR5 safe harbor site, integration at which was mediated by cutting with a Cas9 complexed with established CCR5 gRNA and CCR5 homology arms flanking the AAV integration cassette (Gomez-Ospina, et al. 2019). The integration cassette was comprised of a strong, constitutive SFFV promoter, followed by the FKBP-EPOR coding sequence, then followed by a 2A-YFP-bGH reporter coding sequence.

FIG. 2: Schematic showing insertion of chimeric receptors into HSPCs and screening for BB dimerizer-dependent activation of RBC differentiation in the absence of EPO. Primary human CD34+-enriched HSPCs were plated and expanded in HSC expansion media, and then edited using a CRISPR-Cas/AAV platform to introduce FKBP-EPOR constructs into the HSPCs at the target genomic locus (e.g., the CCR5 safe harbor site). Erythroid differentiation and RBC production were monitored for 16 days in cells from the same donor that were edited as a single treatment in the presence or absence of BB dimerizer (AP20187). Although not indicated in the diagram, day 16 samples exposed to BB dimerizer had many more cells than day 16 samples without exposure to BB dimerizer because erythroid differentiation is associated with dramatic expansion of cells.

FIG. 3: Flow cytometry schematics of WT HSPCs transformed with a mock cassette. Cells were stained at day 14 of RBC differentiation with the standard erythroid differentiation protocol. After using antibodies for CD34, CD45, CD71, and GPA, cells were analyzed by flow cytometry with (right) and without (left) incubation in the presence of BB dimerizer.

FIG. 4: Flow cytometry schematics of WT HSPCs transformed with FKBP-EPOR chimeras 1.4 and 1.5. Cells were stained at day 14 of RBC differentiation with the standard erythroid differentiation protocol. After using antibodies for CD34, CD45, CD71, and GPA, cells were analyzed by flow cytometry with (FIG. 4A) and without (FIG. 4B) incubation in the presence of BB dimerizer.

FIG. 5: Graph showing fold increase in GFP expression in the presence of BB dimerizer relative to the absence of BB dimerizer in live cells containing FKBP-EPOR chimeras 1.4 and 1.5.

FIG. 6: Flow cytometry schematics of second generation of FKBP-EPOR chimeras. Cells were maintained in RBC medium with or without BB, as indicated. FIG. 6A shows the effects of adding a native EPOR signal peptide (SP) to the N-terminus of the receptor encoded by the 1.5 integration cassette (MKC121) and integrating at the CCR5 locus. FIG. 6B shows the effects of adding an IL6 signal peptide to the N-terminus of the receptor encoded by the 1.5 integration cassette (MKC120) and integrating at the CCR5 locus. FIG. 6C shows the effects of a cassette with the same orientation of FKBP and EPOR as 1.5, but using truncated EPOR (tEPOR), integrated at the strong RBC-specific safe harbor locus at HBA (MKC125)(Cromer, et al. Nature Medicine 2021).

FIG. 7: Direct comparison of the flow cytometry schematics of first generation (with cassette 1.5) and second generation (with cassette 2.1) FKBP-EPOR chimeras. Cells were maintained in RBC medium with or without BB dimerizer. To determine the relative potency of adding the IL6 signal peptide, compared to the effective 1.5 chimera from the first-generation vector designs, HSPCs were edited with each vector and analyzed by flow cytometry following RBC differentiation without EPO and with (FIG. 7A) or without BB dimerizer (FIG. 7B).

FIG. 8: Flow cytometry schematics of cells that were edited to contain the IL6-signal peptide containing version of vector 1.5 (FIG. 8A) or the native EPOR signal peptide-containing version of vector 1.5 (FIG. 8B) and kept in HSPC ex vivo culture media without EPO (Dever, et al. Nature 2016) with or without BB dimerizer. Cells were maintained at 100K cells/mL for 7 days post-editing in this media and then stained for the RBC antibody panel.

FIGS. 9 and 10 show flow cytometry schematics of cells which contain an IL6-containing polypeptide with the FKBP extracellular domain attached to a truncated EPOR (tEPOR) integrated at the HBA/locus (MKC135). Cells were incubated without EPO and with (FIGS. 9A and 10A) or without (FIGS. 9B and 10B) BB dimerizer after RBC differentiation without Epo.

FIG. 11 and Table 1 further describe constructs and cell lines used in the experiments described herein.

FIG. 12 shows a summary of data from flow cytometry experiments with some of the edited HSPCs after differentiation in RBC media without EPO and +/−BB dimerizer.

FIG. 13 shows hemoglobin tetramer HPLC data indicating that cells edited with iEPOR version 2.1, 2.2, or 2.3 can produce normal hemoglobin with addition of BB alone equivalent to unedited cells +EPO. Cells edited with iEPOR 2.2 become “leaky” and produce high quantities of hemoglobin even in the absence of EPO and BB.

FIG. 14 illustrates a bicistronic expression vector (MCK144) that combines a beta-thalassemia correction scheme with inducible EPOR expression. This cassette may both correct the molecular pathology of beta-thalassemia and promote RBC differentiation of edited cells.

FIG. 14A shows a diagram of vectors used to edit WT primary human HSPCs. FIG. 14B shows flow cytometry schematics of the differentiated cells incubated with (right) or without (middle) BB dimerizer. A mock sample without a chimeric receptor transgene is shown for comparison on the left.

FIG. 15 depicts the effects of pairing a second-generation RBC drive with a beta-thalassemia correction strategy. FIG. 15A depicts the second-generation vector used in this study. FIG. 15B shows % RBC differentiation of edited cells at day 14 as determined by flow cytometry. The middle and right panels depict % targeted alleles (equivalent to the percentage of edited cells that express RBC markers) as determined by ddPCR in two separate WT HSPC donors over the course of RBC differentiation without EPO and +/−BB dimerizer. The ddPCR assay was set up to quantify the frequency of unedited cells at the integration site compared to a genomic reference probe near the HBA1 locus, which was validated on unedited cells. The inverse of this was assumed to be the frequency of edited cells. FIG. 15C shows hemoglobin tetramer HPLC data indicating BB alone can generate functional RBCs with hemoglobin production equivalent to unedited cells +EPO.

FIG. 16 describes the effects of editing WT human HSPCs that containing inducible chimeric truncated EPOR (IL6-FKBP div.tEPOR) (MKC145) whose coding region was integrated at the endogenous EPOR locus and whose expression is under the control of the EPOR promoter. FIG. 16A depicts the overall scheme that could be used for integrating iEPOR, itEPOR, or div.itEPOR into the endogenous EPOR locus. Div.itEPOR (encoded by SEQ ID NO: 22) is an inducible chimeric receptor with a truncated EPOR that has a codon-diverged sequence relative to native tEPOR due to the incorporation of silent mutations. The presence of silent mutations limits homology to the native sequence so as to prevent premature recombination that would result in incomplete insertion of the integration cassette. As determined by ddPCR and flow cytometry, we observe a BB dimerizer-dependent increase in the number of GFP+ cells in cells with this chimeric transgene integration at the EPOR locus, and many of these are CD34-CD45-CD71+ RBCs (compare FIGS. 16B and 16C).

FIG. 17 shows flow cytometry schematics of WT human HSPCs that were edited with inducible truncated EPOR (IL6-FKBP-tEPOR) whose coding region was integrated at the CCR5 locus and whose expression was under the control of the PGK promoter (MKC158). Although the chimeric protein driven by the PGK promoter appears to be expressed at low levels, there is an observable increase in the overall % of GFP+ cells in the sample with BB dimerizer (FIG. 17A) relative to the sample without BB dimerizer (FIG. 17B), and many of these cells appear to be CD34-CD45-CD71+ RBCs.

FIG. 18 shows HPLC profiles of fetal and adult hemoglobin (HbF and HbA, respectively) within edited and unedited HSPCs either with or without induction. FIG. 18A shows HPLC profile of HbF and HbA from WT unedited umbilical cord blood-derived HSPCs induced with Epo. FIGS. 18B and 18C show hemoglobin HPLC profiles of HSPCs grown with (black) or without (gray) BB dimerizer after editing with the indicated inducible chimeric receptor construct or with a mock cassette. Since some samples had overlapping profiles with and without BB dimerizer (e.g., MKC120 and Mock sample), insets show the HPLC profile of the corresponding sample without BB dimerizer.

FIG. 19 shows bulk RNA-seq results of ~18,000 genes with assigned expression values (as normalized read counts) to compare transcriptional response to native EPOR+EPO vs. iEPOR+BB after genome editing of healthy donor HSPCs and in vitro RBC differentiation. Native EPOR and iEPOR expression levels are annotated for Mock “unedited” cells at dO and d14 of RBC diff (Mock d0 and Mock d14+EPO) followed by cells edited with iEPOR version 2.3 expressed by strong-RBC specific promoter HBA1 (HBA1(iEPOR)+BB), endogenous EPOR promoter (EPOR(iEPOR)+BB), and constitutive-expressing hPGK promoter (PGK(iEPOR)+BB).

FIG. 20 describes IL6-FKBP chimeras that could place SCF, TPOR, and EGFR signaling under control of BB dimerizer. In particular, it depicts three vector cassettes that were cloned and packaged into AAV DNA repair vectors to be used to test other inducible signaling receptor cassettes. The top cassette encodes a chimeric FKBP-SCF-IC receptor. The middle cassette encodes a chimeric FKBP-TPOR-IC receptor. The bottom cassette encodes a chimeric FKBP-EGFR-IC receptor.

FIG. 21 shows flow cytometry schematics of WT primary human HSPCs that were edited with chimeric TPOR and maintained in HSPC media without EPO and with (FIG. 21B) or without (FIG. 21C) BB dimerizer for 7 days post-editing relative to a mock control (FIG. 21A). Cells were stained with an HSC-specific antibody panel developed by the Ravindra Majeti lab at Stanford, which includes markers for CD34, CD90, and CD123.

FIG. 22 summarizes flow cytometry schematics for WT primary human HSPCs that were edited to express an inducible chimeric TPOR (IL6-FKBP-TPOR or iTPOR) receptor or an inducible chimeric SCF (IL6-FKBP-SCF or iSCF) receptor and maintained in HSPC media with and without BB dimerizer for 7 days post-editing. FIG. 22A shows a BB-dependent increase in GFP expression in cells edited with either an inducible chimeric TPOR receptor or an inducible chimeric SCF receptor relative to cells edited with a mock control. FIGS. 22B and 22C show the targeted allele frequency for cells edited with either the BB-inducible chimeric TPOR receptor or the BB-inducible chimeric SCF receptor, respectively.

FIG. 23 shows editing efficiency of the PGK-driven iEPORv2.3 in iPSCs with or without AZD, a small molecule used to increase genome editing frequency, indicating high editing rates with the PGK-driven iEPORv2.3 integration cassette in human iPSCs.

FIG. 24A depicts an iPSC>HSC>RBC differentiation workflow using STEMdiff Hematopoietic Kit. FIG. 24B shows no major differences in cell proliferation between unedited cells cultured with BB (+BB) and iEPOR-edited cells cultured without BB (−BB) at the first stage of differentiation (iPSC>HPC). FIG. 24C shows a dramatic increase of RBC differentiation in iEPOR-edited cells cultured +BB during stage 1 iPSC>HPC, indicating that some frequency of early differentiation driven by BB-induced iEPOR signaling is occurring at this stage.

FIG. 25A depicts an iPSC>HSC>RBC differentiation workflow using in-house differentiation protocol for CD34s. FIG. 25B shows iEPOR-edited cells cultured with BB but without EPO (−EPO/+BB) lead to an intermediate cell expansion in comparison to cells cultured with EPO over the second stage of differentiation (HPC>RBC), indicating that BB can be used to replace EPO in the media and lead to substantial RBC production. FIG. 25B also shows the highest cell production in iEPOR-edited cells cultured with EPO and BB (+EPO/+BB), indicating that BB stimulation of iEPOR leads to an elevated boost even on top of natural EPO stimulation.

FIG. 26 shows RBC proliferation and differentiation differences between cells with or without EPO, indicating that in absence of EPO, only iEPOR-edited cells with BB (+BB) are able to effectively differentiate and proliferate.

### DETAILED DESCRIPTION

### 1. Introduction

The present disclosure provides methods and compositions for tunable differentiation of a hematopoietic stem and progenitor cell (HSPC) in response to a small molecule. In some instances, this is an FDA-approved orally bioavailable small molecule, e.g., AP20187, also referred to as “B/B homodimerizer,” or “BB dimerizer.” The methods and compositions use a CRISPR-Cas system to introduce into cells a chimeric transmembrane receptor polypeptide that dimerizes in response to an extracellular dimerization signal. Dimerization triggers a signaling cascade, the consequence of which is increased survival, increased proliferation, and/or increased erythroid differentiation.

In certain embodiments, the HSPC is also genetically modified using a CRISPR-Cas system to correct an allele that causes a genetic disorder. In those instances, the methods and compositions described herein may provide an improved means for treating a genetic disorder. Current treatments for genetic disorders using edited HSPCs require a devastating mycloablation regimen to clear out the bone marrow in order for edited HSPCs to sufficiently engraft. The methods and compositions described herein allow for selective differentiation of edited HSPCs such that lower levels of engraftment are sufficient to treat a disorder. Therefore, the methods and compositions described herein overcome at least one significant recognized obstacle to treating genetic disorders.

### 2. General

Practicing this disclosure utilizes routine techniques in the field of molecular biology. Basic texts disclosing the general methods of use in this disclosure include Sambrook and Russell, Molecular Cloning. A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Let. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a.” “an,” and “the” include plural referents unless the context clearly dictates otherwise. Thus, for example, reference to “a cell” includes a plurality of such cells, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1,04X, 1.05X, 1.06X, 1.07X, 1.08X, 1.09X, 1.1X, 1.11 X, 1.12X, 1.13X, 1.14X, 1.15X, 1.16X, 1.17X, 1.18X, 1.19X, and 1.2X. Thus, “about X” is intended to teach and provide written description support for a claim limitation of, e.g.. “0.98X.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

An “expression cassette” or “cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a first polynucleotide or polypeptide is “heterologous” to an organism or a second polynucleotide or polypeptide sequence if the first polynucleotide or polypeptide originates from a foreign species compared to the organism or second polynucleotide or polypeptide, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “expression” and “expressed” refer to the production of a transcriptional and/or translational product, e.g., of a tEPOR encoding mRNA or an encoded tEPOR protein. In some embodiments, the term refers to the production of a transcriptional and/or translational product encoded by a gene or a portion thereof. The level of expression of a DNA molecule in a cell may be assessed on the basis of either the amount of corresponding mRNA that is present within the cell or the amount of protein encoded by that DNA produced by the cell.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants of a protein can have an increased stability, assembly, or activity as described herein.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (l), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93%, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J Mol. Biol. 215: 403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues: always <0). For amino acid sequences, a scoring matrix is used to calculate the cumulative score. Extension of the word hits in each direction are halted when: the cumulative alignment score falls off by the quantity X from its maximum achieved value: the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff& Henikoff, Proc. Natl. Acad Sci. USA 89:10915 (1989)).

The BLAST algorithm also performs a statistical analysis of the similarity between two sequences (see, e.g., Karlin & Altschul, Proc. Nat'l. Acad. Si. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acids. CRISPR-Cas systems are found in a wide range of bacterial and archacal organisms. CRISPR-Cas systems fall into two classes with six types, I, II, III, IV, V, and VI as well as many sub-types, with Class 1 including types I and III CRISPR systems, and Class 2 including types II, IV, V and VI: Class 1 subtypes include subtypes I-A to I-F, for example. See, e.g., Fonfara et al., Nature 532, 7600 (2016): Zetsche et al., Cell 163, 759-771 (2015): Adli et al. (2018). Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are affected by multiple Cas proteins, with Cas3 providing the endonuclease activity, whereas in class 2 systems they are all carried out by a single Cas, Cas9.

A “homologous repair template” or “donor template” refers to a polynucleotide sequence that can be used to repair a double stranded break (DSB) in the DNA. e.g., a CRISPR/Cas9-mediated break at a CCR5 locus as induced using the herein-described methods and compositions. The homologous repair template comprises homology to the genomic sequence surrounding the DSB, i.e., comprising CCR5 homology arms. In particular embodiments, two distinct homologous regions are present on the template, with each region comprising at least 50, 100, 200, 300, 400, 500, 600, 700, 800, 900, 400-1000, 500-900, or more nucleotides of homology with the corresponding genomic sequence. In particular embodiments, the templates comprise two homology arms comprising, e.g., about 900 nucleotides of homology, with one arm extending upstream starting at the translation start site, and the other arm extending downstream from the sgRNA target site. The repair template can be present in any form. e.g., on a plasmid that is introduced into the cell, as a free floating doubled stranded DNA template (e.g., a template that is liberated from a plasmid in the cell), or as single stranded DNA. In particular embodiments, the template is present within a viral vector, e.g., an adeno-associated viral vector such as AAV6. The templates of the present disclosure can also comprise a transgene, e.g., a chimeric transmembrane receptor transgene and optionally a therapeutic transgene as described herein.

As used herein, “homologous recombination” or “HR” refers to insertion of a nucleotide sequence during repair of double-strand breaks in DNA via homology-directed repair mechanisms. This process uses a “donor template” or “homologous repair template” with homology to nucleotide sequence in the region of the break as a template for repairing a double-strand break. The presence of a double-stranded break facilitates integration of the donor sequence. The donor sequence may be physically integrated or used as a template for repair of the break via homologous recombination, resulting in the introduction of all or part of the nucleotide sequence. This process is used by a number of different gene editing platforms that create the double-strand break, such as meganucleases, such as zinc finger nucleases (ZFNs), transcription activator-like effector nucleases (TALENs), and the CRISPR-Cas9 gene editing systems. In particular embodiments. HR involves double-stranded breaks induced by CRISPR-Cas9.

EPOR (erythropoietin receptor) is the receptor for erythropoietin (EPO), a cytokine that regulates the proliferation and differentiation of erythroid precursor cells. When italicized (i.e., EPOR), EPOR refers to a polynucleotide (e.g., gene, locus, transgene, coding sequence, cDNA, expression cassette) encoding EPOR. Upon binding of EPO, EPOR activates JAK2 tyrosine kinase, which in turn activates different intracellular pathways such as Ras/MAP kinase, PI3 kinase, and STAT transcription factors. EPOR is a member of the cytokine receptor family, and the EPOR gene is located on human chromosome 19p (19p 13.2). The NCBI gene ID for human EPOR is 2057, and the UniProt ID for human EPOR is P19235, the entire disclosures of which are herein incorporated by reference. The reference WT EPOR cDNA sequence used here is shown in SEQ ID NO: 20.

Truncated EPOR, or tEPOR (encoded by tEPOR), refers to forms of the EPO receptor, or to polynucleotides encoding the receptor forms, that lack a portion or all of the receptor's cytoplasmic domain. For example, in some embodiments a tEPOR lacks the 70 C-terminal amino acids of full-length EPOR. In some embodiments, a tEPOR lacks all 236 amino acids of the cytoplasmic domain. In some embodiments, a tEPOR lacks, e.g., about 10, 20, 30, 40, 50, 60, 70, 80, 90, 100, 110, 120, 130, 140, 150, 160, 170, 180, 190, 200, 210, 220, 230, 10-236, 10-50, 50-60, 60-70, 65-75, 70-80, 80-90, 90-100, 100-150, 150-200, or 200-236 amino acids. In some embodiments, a tEPOR lacks a binding site and/or does not interact with the tyrosine phosphatase SHP-1 (or SHPTP-1), which normally plays a role in inhibiting EPOR signaling. In some embodiments, a coding sequence (e.g., gene or transgene) encoding a tEPOR comprises a nonsense mutation in exon 7 or exon 8, and/or encodes any of the herein-described forms of truncated EPOR. Nonsense mutations causing the expression of truncated EPOR act as dominant mutations that render cells hypersensitive to EPO, leading to an ability to undergo effective proliferation and differentiation in the presence of reduced amounts of EPO, and to show enhanced levels of proliferation and differentiation in the presence of normal EPO levels. An exemplary tEPOR cDNA is shown herein as SEQ ID NO: 21. As used herein, tEPOR can refer to any nucleotide sequence comprising about 70%, 75%, 80%, 85%, 90%, 95%, 96%, 97%, 98%, 99%, or more homology to SEQ ID NO: 21 or a subsequence thereof.

“iEPOR” and “itEPOR” proteins comprise a signal peptide (e.g., IL6) and FKBP and EPOR polypeptides, or fragments thereof, in the same orientation as FKBP and EPOR polypeptides in 1.5. Similarly, “iTPOR” and “iSCF” are used to describe proteins comprising a signal peptide and the orientation of FKBP and EPOR polypeptides as in 1.5, with polypeptides from TPOR or SCF instead of EPOR.

As used herein, the terms “hematopoietic stem and progenitor cell” and “HSPC” refer to a hematopoietic stem cell (HSC), a hematopoietic progenitor cell (HPC), or a population of hematopoictic stem cells and hematopoictic progenitor cells.

### 4. Designing Chimeric Transmembrane Receptor Polypeptides

In one aspect, the present disclosure provides a chimeric transmembrane receptor polypeptide comprising: an extramembrane dimerizer domain, wherein the extramembrane dimerizer domain induces dimerization of the chimeric transmembrane receptor polypeptide upon recognition of a dimerization signal; a transmembrane domain; and an intramembrane domain, wherein the intramembrane domain is configured to induce activation of one or more intramembrane signal pathways upon dimerization of the chimeric transmembrane receptor polypeptide in a modified primary human cell comprising the chimeric transmembrane receptor polypeptide, and wherein the one or more intramembrane signaling pathways promote survival, proliferation, and/or differentiation of the modified primary human cell. A chimeric polypeptide may, for example, comprise fragments of distinct proteins and contain functional properties derived from each of the distinct proteins.

In some embodiments, the dimerization signal is a pharmaceutically acceptable small molecule dimerization signal. In some embodiments, the dimerization signal is an orally bioavailable small molecule. In some embodiments, the small molecule dimerization signal comprises AP20187, AP21967, ganciclovir, rapamycin or an analog thereof. In some embodiments, the small molecule dimerization signal comprises AP20187. AP20187 is a cell permeable small molecule approved by the FDA that is orally bioavailable and can be used to induce dimerization of FK506-binding protein (FKBP).

In some embodiments, the extramembrane dimerizer domain comprises an FKBP domain, or a variant or a fragment thereof. The chimeric transmembrane receptor polypeptide extramembrane domain can include or consist of a heterodimer FKBP/FRB domain. As used herein, the terms “variant,” and “fragment,” refer to a polypeptide related to a wild-type polypeptide, for example, either by amino acid sequence, structure (e.g., secondary and/or tertiary), activity (e.g., enzymatic activity) and/or function. Variants and fragments of a polypeptide can include one or more amino acid variations (e.g., mutations, insertions, and deletions), truncations, modifications, or combinations thereof compared to a wild-type polypeptide. A variant or fragment can include at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the sequence, structure, activity, and/or function of the corresponding wild-type polypeptide. In some embodiments, the extramembrane dimerizer domain comprises an mFRB domain, an HSV-TK dimerization domain, a rapamycin-inducible dimerization domain, a rapalogue-inducible dimerization domain, or a combination thereof. As disclosed herein, the mFRB domain can heterodimerize with the FKBP domain using AP21967; the HSV-TK domain can homodimerize with ganciclovir; and the FKBP domain can homodimerize with AP20187, AP21967, rapamycin or its various analogs (i.e., rapalogs).

In some embodiments, the intramembrane domain comprises an EPOR intracellular domain, a stem cell factor (SCF) intracellular domain or a myeloprolifereatie leukemia virus oncogene (MPL) intracellular domain, a thrombopoietin (TPOR) intracellular domain or a CD117 intracellular domain, an epidermal growth factor (EGFR) intracellular domain, an RET intracellular domain, a macrophage colony-stimulating factor 1 receptor (CSF1R) intracellular domain, an insulin-like growth factor (IGF1R) intracellular domain, or a combination thereof.

In some embodiments, the intramembrane domain comprises an EPOR intracellular domain and wherein dimerization of the chimeric transmembrane receptor polypeptide induces activation of intramembrane signal pathways resulting in increased survival, increased proliferation, and/or increased erythroid differentiation of the modified primary human cell upon recognition of the dimerization signal. In some embodiments, the intramembrane domain comprises a TPOR intracellular domain and wherein dimerization of the chimeric transmembrane receptor polypeptide induces activation of intramembrane signal pathways resulting in increased survival, increased proliferation, and/or increased erythroid differentiation of the modified primary human cell upon recognition of the dimerization signal. In some embodiments, the intramembrane domain comprises an SCF intracellular domain and wherein dimerization of the chimeric transmembrane receptor polypeptide induces activation of intramembrane signal pathways resulting in increased survival, increased proliferation, and/or increased erythroid differentiation of the modified primary human cell upon recognition of the dimerization signal.

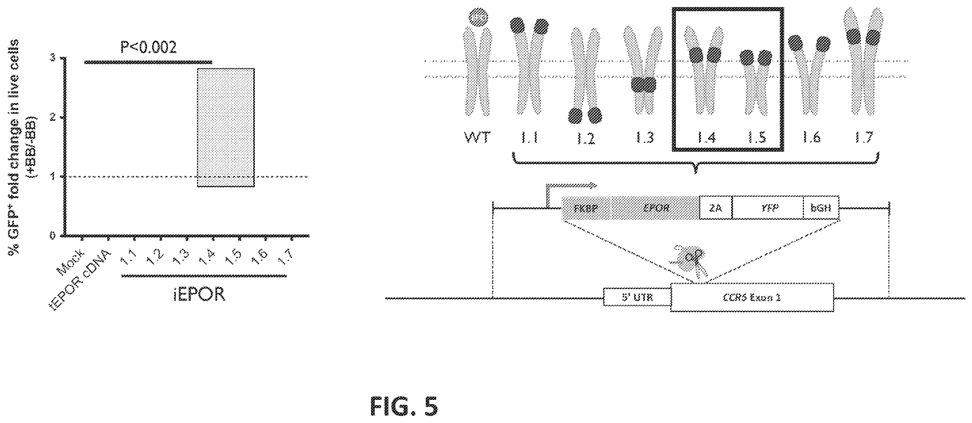

In some embodiments, the extramembrane dimerizer domain is immediately adjacent to the transmembrane domain. As shown in FIG. 5, only a subset of the chimeric FKBP-EPOR polypeptides (1.4 and 1.5) caused BB-induced differentiation of HSPCs. Unexpectedly, both of these chimeric polypeptides contained an extracellular dimerizer domain immediately adjacent to the transmembrane domain. For 1.4 and 1.5, the immediately adjacent domains are separated by only a linker peptide sequence, but in other contexts immediately adjacent domains may contain multiple linker peptide sequences, e.g., two linker peptide sequences, three linker peptide sequences, four linker peptide sequences, five linker peptide sequences, or six linker peptide sequences.

Construct 1.5 was subsequently used to create second generation constructs since it is a smaller cassette, which can be more easily included in an AAV repair template, and it fully removes the extracellular domain of EPOR (as indicated in FIG. 5), ensuring that it is no longer responsive to EPO protein.

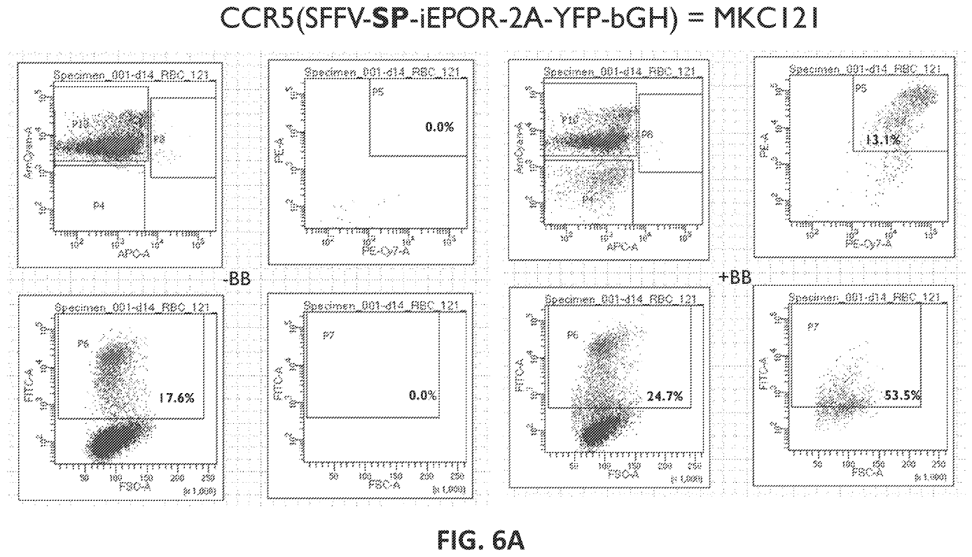

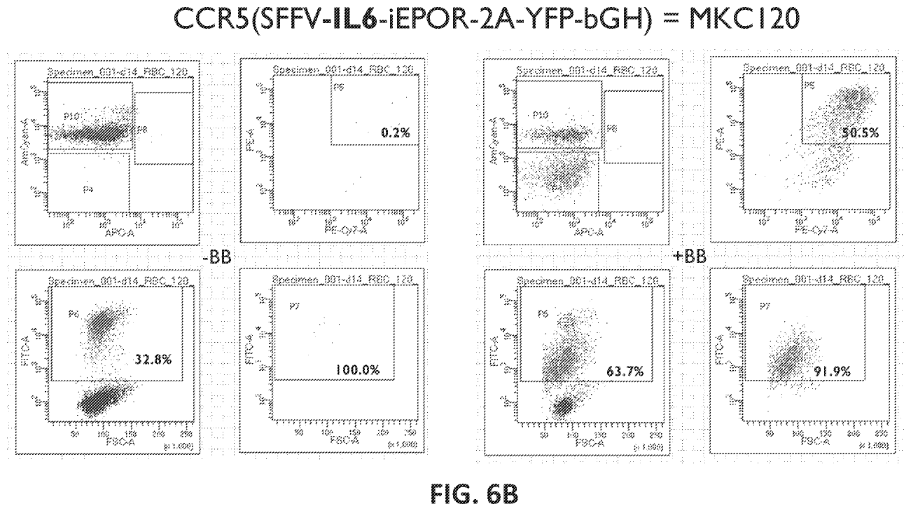

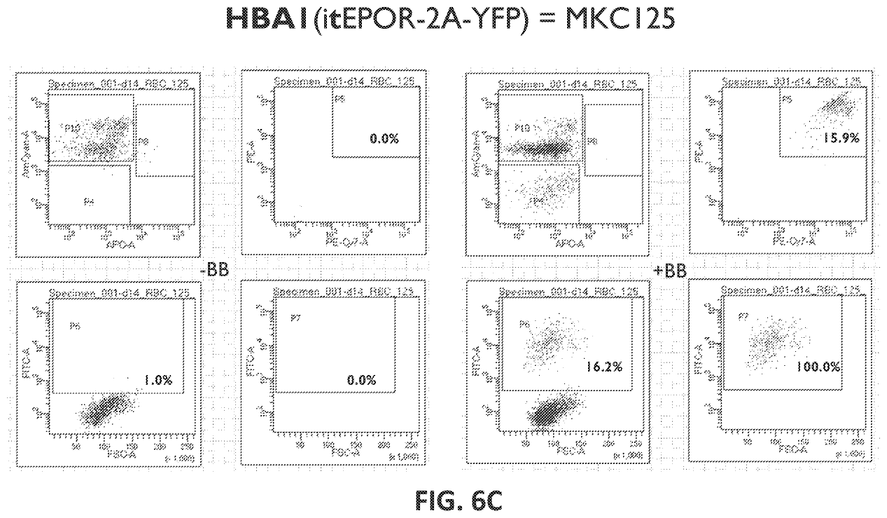

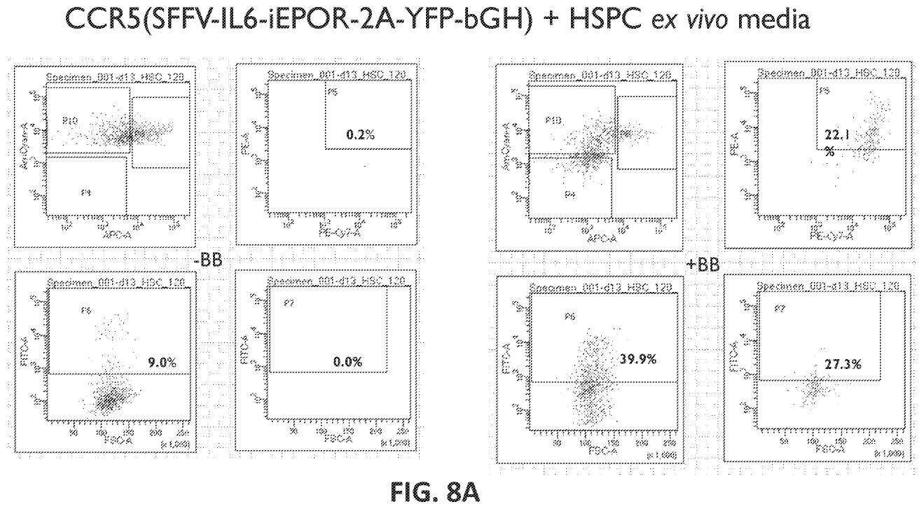

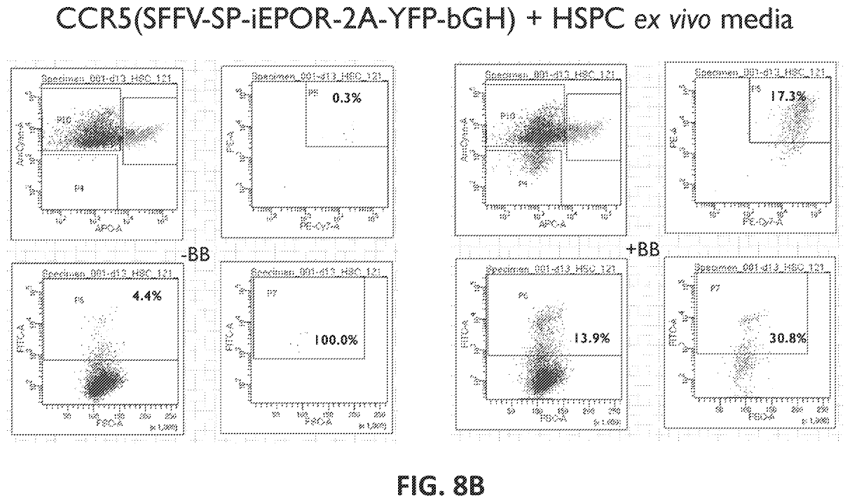

In some embodiments, the chimeric transmembrane receptor polypeptide further comprises a signal peptide. In some embodiments, the signal peptide promotes cell surface membrane localization of the chimeric transmembrane receptor polypeptide. In some embodiments, the signal peptide comprises an IL6 signal peptide (such as SEQ ID NO: 4), an EPOR signal peptide (such as SEQ ID NO: 5), a lysozyme C signal peptide, an angiotensinogen signal peptide, an RNASE1 signal peptide, an RNASE3 signal peptide, or a modified human albumin signal peptide. Both the IL-6 and EPOR signal peptides (indicated as SP) enhanced BB-dependent differentiation as shown in FIGS. 6A and 6B. Surprisingly, these constructs were able to induce BB-driven differentiation in Porteus HSPC ex vivo culture media (media known to preserve HSPC stem-ness: Dever et al., Nature 2016), as shown in FIGS. 8A and 8B. This suggests that this is a strong, pro-RBC differentiation strategy that is activated by BB dimerizer.

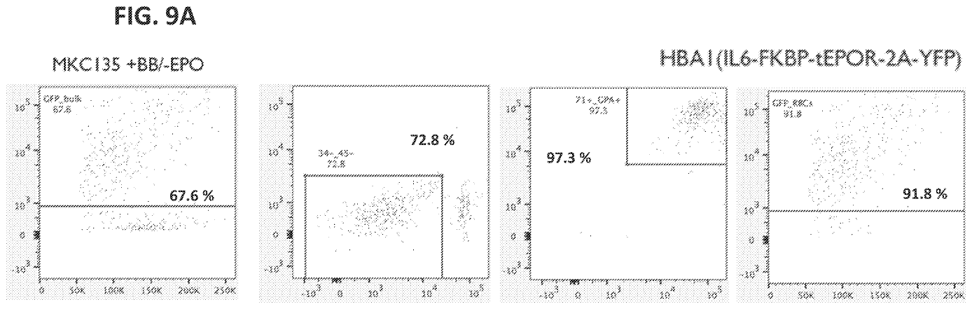

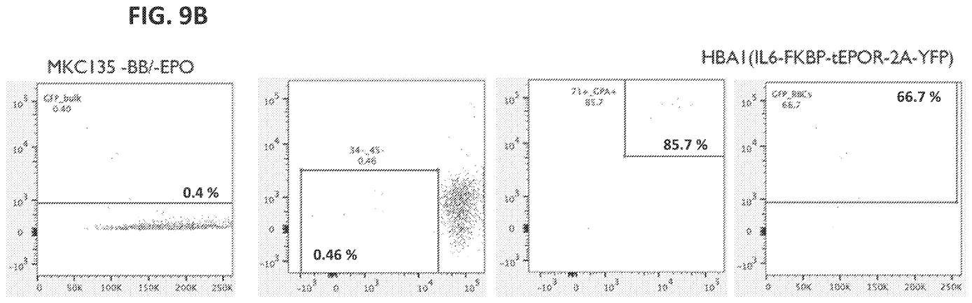

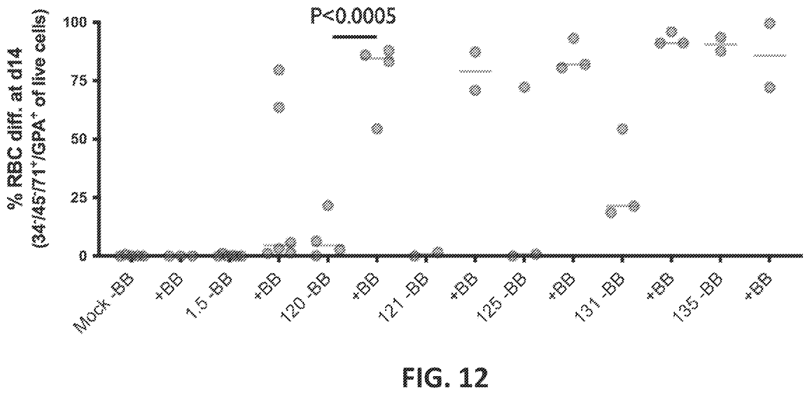

Combining optimization strategies created a single editing vector which integrates an IL-6-containing FKBP-tEPOR construct at the HBA1 locus. The resulting construct was so potent that the cell line (MKC135) exhibited BB-independent RBC differentiation, as described in FIG. 9B and FIG. 12. This may be due to the fact that expression of an iEPOR (IL-6-FKBP-EPOR) from the HBA1 locus results in supraphysiological levels of this receptor translocating to the cell surface. As a consequence, these chimeric EPOR monomers may incidentally collide and activate some degree of EPOR signaling even in the absence of BB dimerizer. This demonstrates that optimizations can achieve extremely high-functioning signaling receptors.

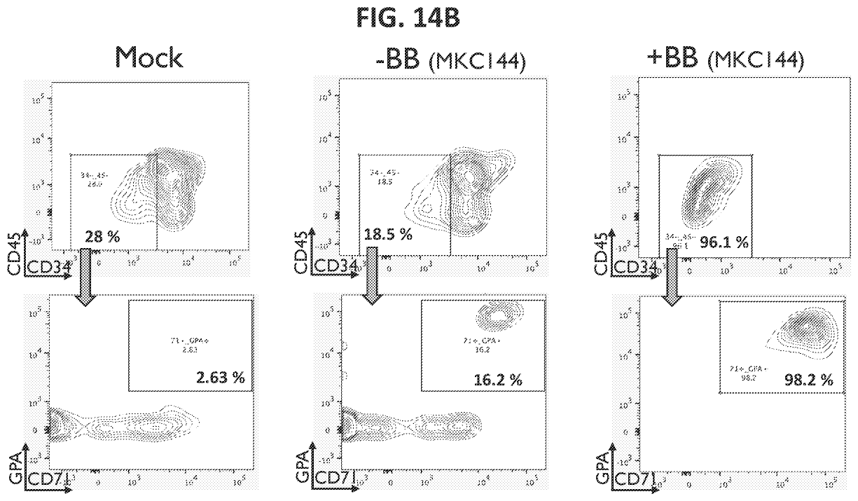

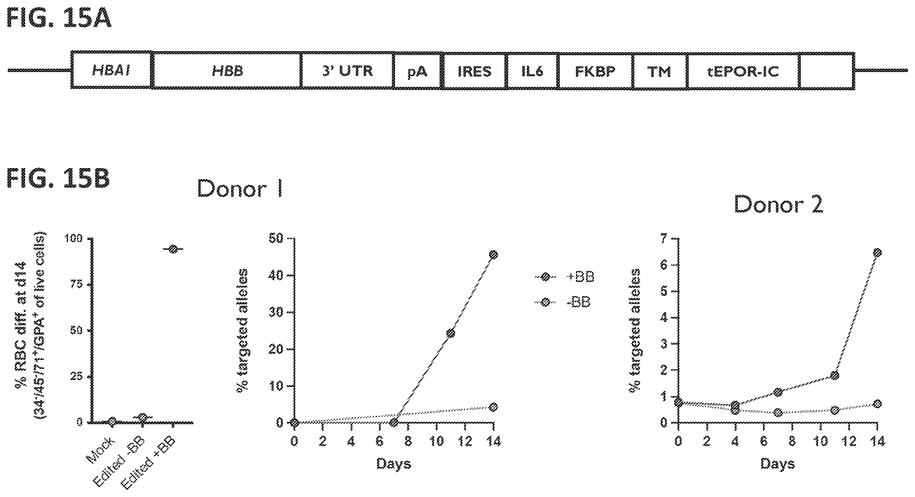

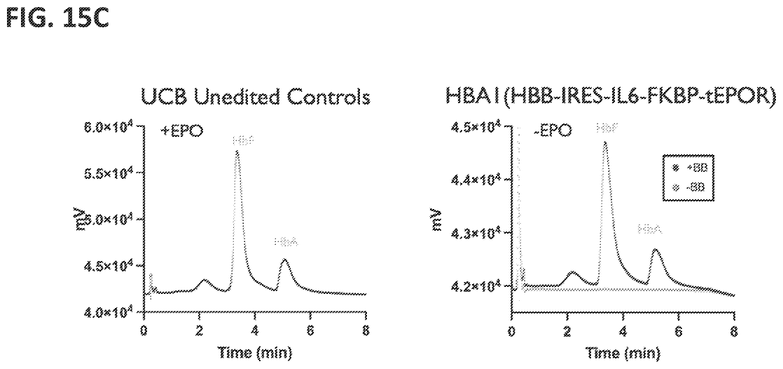

Dialing back expression and reducing the hyper-function of this chimeric receptor could restore BB dimerizer-dependent control. One such way to do so is using a bicistronic vector that integrates both the insertion of a beta-globin gene and the itEPOR (IL-6-FKBP-tEPOR) cassette at the HBA1 locus. This is shown in FIG. 14B, using the construct in MKC144. Strong BB-dependent differentiation was observed after editing and differentiation of these HSPCs. As shown in FIG. 15B, editing frequencies were quantified by ddPCR, revealing BB-dependent enrichment of edited cells. There was a dramatic increase in the overall proportion of edited cells in the bulk population of cells.

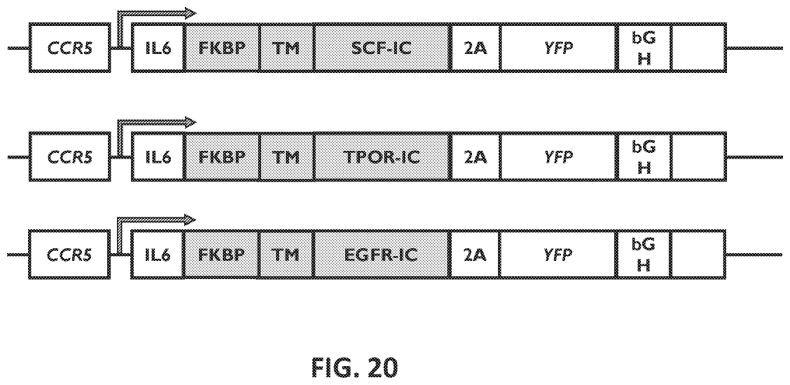

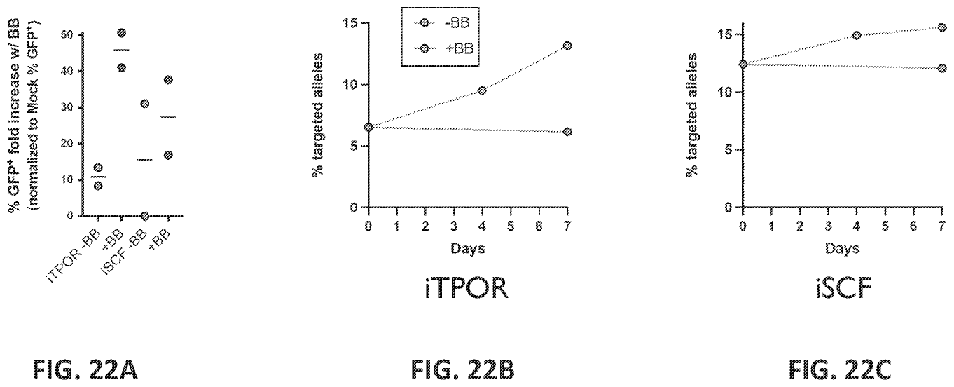

BB-dependent differentiation was also observed after editing and differentiation of HSPCs with constructs comprising iSCF (IL-6-FKBP-SCF) and iTPOR (IL-6-FKBP-TPOR) cassettes, as shown in FIGS. 20, 21A-21C and 22A. As expected, the proportion of the cells carrying the edited or targeted allele increased over time, as shown by ddPCR in FIGS. 22B and 22C.

In some embodiments, the chimeric transmembrane receptor polypeptide further comprises a linker peptide. The provided chimeric transmembrane receptor polypeptide can optionally include one or more linker peptide sequences. In some embodiments, the chimeric transmembrane receptor polypeptide includes two linker peptide sequences. In some embodiments, the receptor polypeptide includes more than two linker peptide sequences.

Linker peptide sequences can be found, for example, between elements of the chimeric proteins described herein. For instance, one or more linker peptide sequences may connect signal peptides to the proteins (e.g., signal peptide-linker-1.5), as well as FKBPs to EPOR in various orientations. In designs such as 1.4, which have an FKBP in the middle of EPOR, one or more linker peptide sequences can be incorporated on each end of the FKBP.

In some embodiments, the linker peptide comprises the amino acid sequence GGGGS. Linker sequences suitable for use with the provided chimeric transmembrane receptor polypeptide include, for example, those consisting of glycine (G) and serine (S). In some embodiments, at least one of the one or more linker peptide sequences of the provided chimeric transmembrane receptor polypeptide is a GGS linker peptide sequence. In some embodiments, each of the one or more linker peptide sequences is GGS. In some embodiments, at least one of the one or more linker peptide sequences of the chimeric transmembrane receptor polypeptide is a GGSGGSGGS linker peptide sequence. In some embodiments, each of the one or more linker peptide sequences is GGSGGSGGS. In some embodiments, at least one of the one or more linker peptide sequences of the chimeric transmembrane receptor polypeptide is a GS linker sequence. In some embodiments, each of the one or more linker peptide sequences is GS. In some embodiments, at least one of the one or more linker peptide sequences of the chimeric transmembrane receptor polypeptide is a GSGSGS linker peptide sequence. In some embodiments, each of the one or more linker peptide sequences is GSGSGS.

Other linker peptide sequences suitable for use with the provided chimeric transmembrane receptor polypeptide include, for example, IgG hinge linker peptide sequences. In some embodiments, at least one of the one or more linker peptide sequences of the chimeric transmembrane receptor polypeptide is a wild-type IgG4 ESKYGPPCPPCP linker peptide sequence. In some embodiments, each of the one or more linker peptide sequences is ESKYGPPCPPCP. In some embodiments, at least one of the one or more linker peptide sequences of the chimeric transmembrane receptor polypeptide is a mutated IgG4 ESKYGPPAPPAP linker peptide sequence. In some embodiments, each of the one or more linker peptide sequences is ESKYGPPAPPAP.

In some embodiments, the chimeric transmembrane receptor polypeptide comprises an amino acid sequence having at least 80% identity to any one of SEQ ID NOS: 1 to 3 or 7 to 11.

In another aspect, the present disclosure provides a recombinant nucleic acid encoding any of the chimeric transmembrane receptor polypeptide described herein.

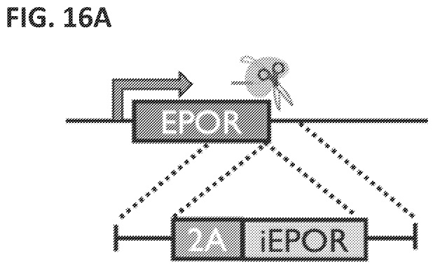

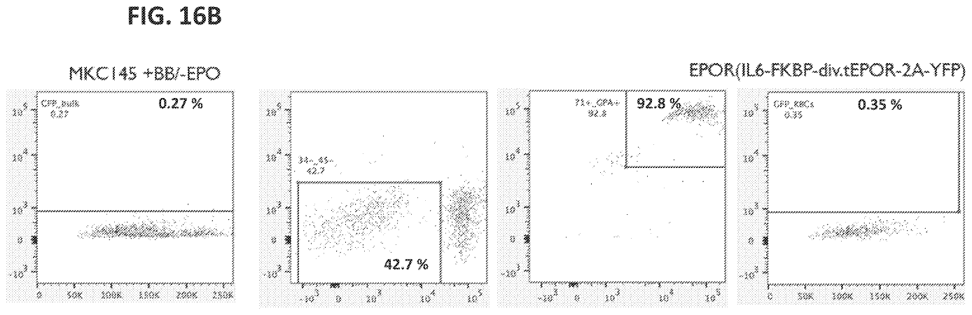

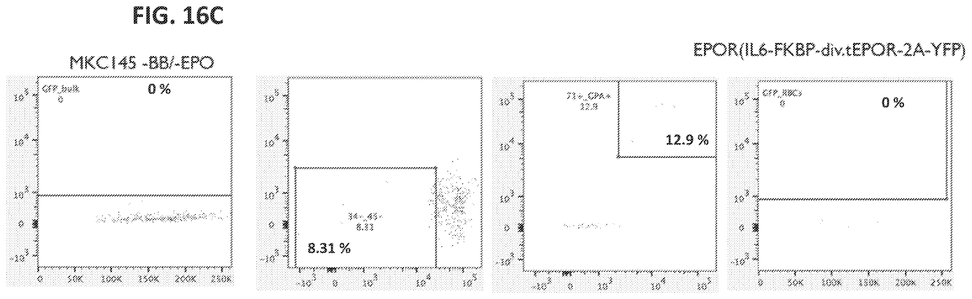

In another aspect, the present disclosure provides a DNA construct comprising a promoter operably linked to the recombinant nucleic. In some embodiments, the promoter is an endogenous EPOR promoter, an endogenous HBA1 promoter, an endogenous TPOR promoter, a constitutive SFFV promoter, a constitutive PGK promoter, or a constitutive UbC promoter. Integration at the HBA1 locus is predicted to elicit strong, erythroid-specific expression, and did show enhanced BB-dependent expression of inducible chimeric tEPOR, as shown in FIG. 6C. Of course, the overall expression of the transgene can vary depending on the promoter used to express the transgene. For example, as shown in FIGS. 16A and 16B, integration at the CCR5 locus of an inducible truncated EPOR construct that uses the PGK promoter to express the transgene (PGK-IL6-FKBP-tEPOR) results in an observable increase in GFP+ edited cells, of which many are CD34-CD45-CD71+ RBCs. However, the GFP+ signal is at low levels for these samples even in the presence of BB dimerizer compared to some of the samples that use other inducible chimeric transgenes.

Also provided is a vector comprising any of the recombinant nucleic acids described herein or any of the DNA constructs described herein.

In another aspect, the present disclosure provides a host cell comprising any of the recombinant nucleic acids described herein, any of the DNA constructs described herein, or any of the vectors described herein. In some embodiments, the recombinant nucleic acid, DNA construct, or vector is integrated into the EPOR locus, the CCR5 locus or the HBAJ locus. In some embodiments, the inducible chimeric receptor coding region is integrated at the 3′ end of the endogenous EPOR locus. One such scheme is depicted in FIG. 16A and involves cutting near the endogenous EPOR gene and inserting the inducible chimeric transgene. In order to prevent premature recombination that would result in incomplete insertion the iEPOR gene or fragment thereof, the cDNA encoding the iEPOR gene or fragment thereof could be codon-diverged relative to the native EPOR sequence by incorporating silent mutations. For example, integration of an inducible codon-diverged truncated EPOR receptor transgene (IL6-FKBP-div.tEPOR) resulted in an obeservable BB dimerizer-dependent increase in GFP+ cells relative to the samples without BB dimerizer (compare FIGS. 16B and 16C). Furthermore, the GFP+ cells appear to be CD34-CD45-CD71+ RBCs.

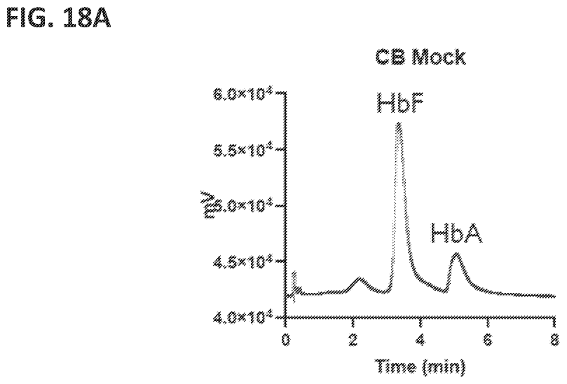

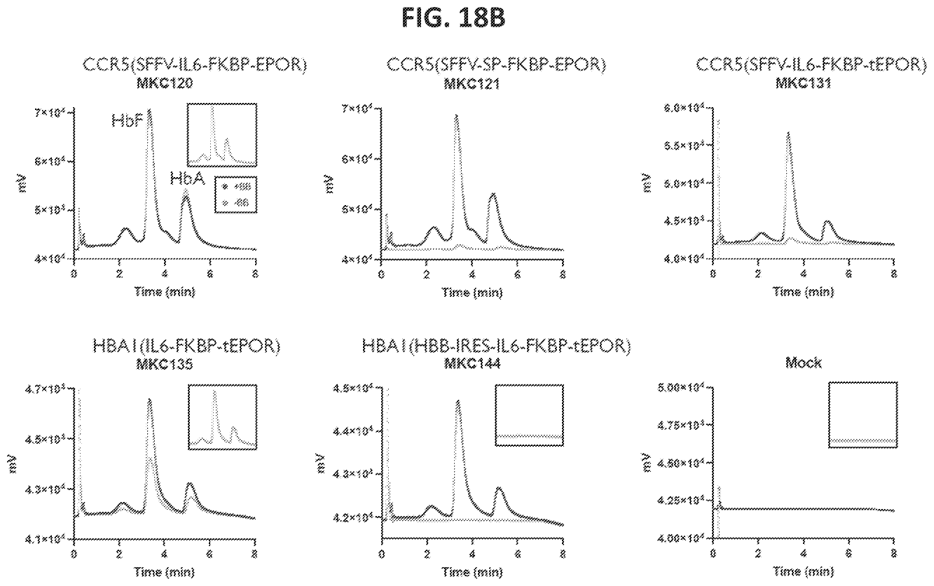

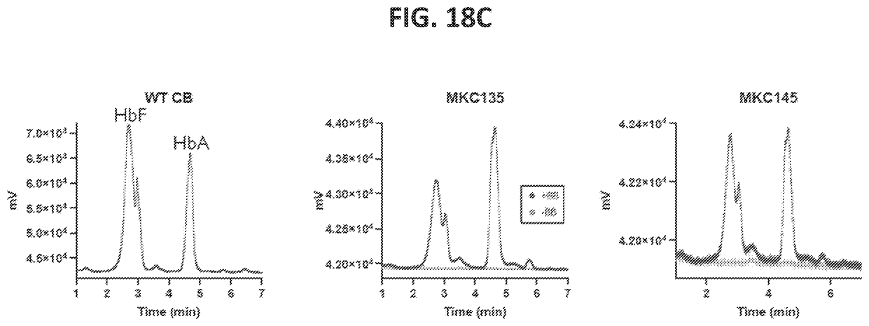

In some embodiments, the RBCs produced by cells containing inducible chimeric transgenes are functionally normal in terms of hemoglobin production. FIG. 18A shows the profile of fetal hemoglobin (HbF) and adult hemoglobin (HbA) in unedited umbilical cord blood-derived HSPCs (CB Mock) that were differentiated in RBC media with Epo.

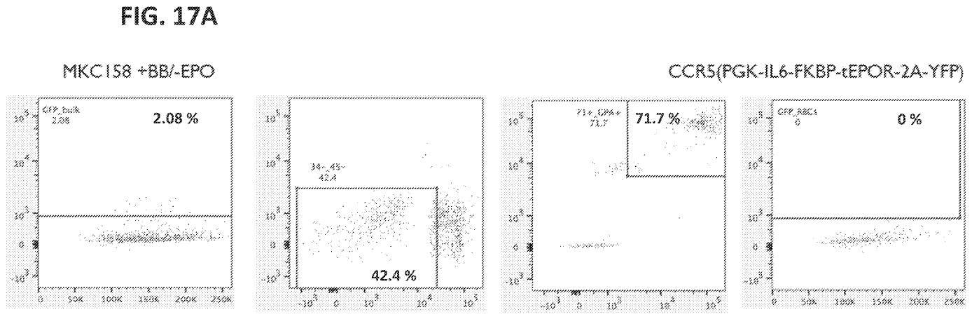

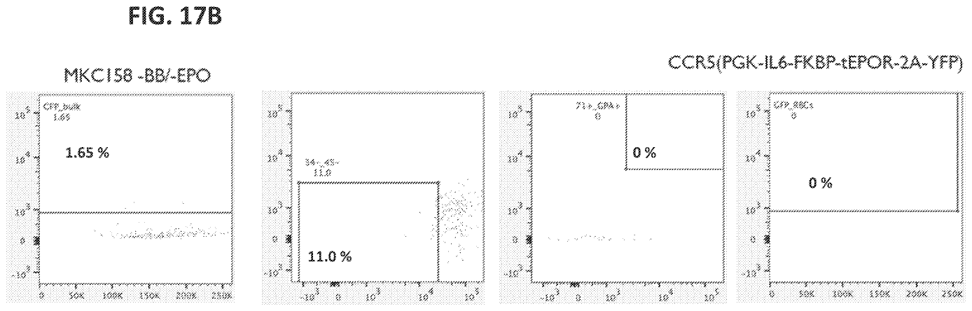

The HbF and HbA profiles in edited cells containing integrated chimeric receptor transgenes with BB dimerizer, displayed HbF and HbA production that is equivalent in total amount and ratio of HbF:HbA to the CB Mock sample with EPO (compare FIGS. 17B and 17C to 17A). For cells edited with MKC121, MKC131, and MKC144 and then incubated without BB dimerizer, the hemoglobin profiles closely resembled those from cells with a mock cassette without either EPO or BB dimerizer. Therefore, in some instances, the native EPO signaling is replaced with BB-inducible signalling to create functionally normal RBCs.

In some embodiments, the host cell is a eukaryotic cell. In some embodiments, the host cell is a primary human cell. In some embodiments, the host cell is a hematopoietic stem cell, a hematopoietic progenitor cell, a T cell, a B cell, an airway basal stem cell, or a pluripotent stem cell (PSC). In some embodiments, the host cell was derived from a patient who is a carrier of an allele that causes a genetic disorder. In some embodiments, the genetic disorder is beta-thalassemia, sickle cell disease (SCD), severe combined immunodeficiency (SCID), mucopolysaccharidosis type 1, Cystic Fibrosis, Gaucher disease, Krabbe disease, X-linked chronic granulomatous disease (X-CGD), or a combination thereof. In some embodiments, the genetic disorder is a hemoglobinopathy. In some instances, the hemoglobinopathy is beta-thalassemia or sickle cell disease (SCD). In some embodiments, the genome of the host cell is edited to contain a corrected version of the allele that causes the genetic disorder.

In another aspect, the present disclosure provides a method of inducing erythroid differentiation of an HSPC, the method comprising contacting an HSPC that comprises a recombinant nucleic acid encoding a chimeric transmembrane receptor polypeptide with the dimerization signal.

In another aspect, the present disclosure provides a method of tuning red blood cell levels in a patient, the method comprising: (i) editing an HSPC so that it can express at least one chimeric transmembrane receptor polypeptide described herein; (ii) transferring the resulting HSPC to the patient; (iii) administering a first quantity of the dimerization signal to the patient: (iv) monitoring red blood cell levels in the patient; and (v) administering a second quantity of the dimerization signal to the patient that is the same, less, or more than the first quantity of dimerization signal.

In another aspect, the present disclosure provides a method of increasing the proportion of red blood cells with a corrected version of an allele that causes a genetic disorder, the method comprising: (i) creating an edited HSPC by introducing a corrected version of the allele associated with a genetic disorder introducing a construct that encodes at least one chimeric transmembrane receptor polypeptide described herein: (iii) transferring the edited HSPC to the patient; and (iv) administering the dimerization signal to the patient. In some embodiments, the HSPC is derived from the patient's own cells. In some embodiments, the HSPC is derived from an allogeneic donor's cells. In some embodiments, the genetic disorder is beta-thalassemia, sickle cell disease (SCD), severe combined immunodeficiency (SCID), mucopolysaccharidosis type 1, Cystic Fibrosis, Gaucher disease, Krabbe disease. X-linked chronic granulomatous disease (X-CGD), or a combination thereof.

### 5. CRISPR/Cas Systems

The present disclosure uses CRISPR guide sequences that specifically direct the cleavage of a EPOR, CCR5, HBA1, or HBB gene by RNA-guided nucleases, in particular within coding sequences encoding the EPOR cytoplasmic domain. The present disclosure provides a CRISPR-Cas/AAV6-mediated genome editing method that can achieve high rates of targeted integration at these loci.

The chimeric transmembrane receptor transgene may encode a fragment of a extramembrane domain from one cell surface receptor and a fragment of an intracellular domain from the same or a different cell surface receptor. For example, the chimeric transmembrane receptor transgene may comprise the coding region for a fragment of FKBP and either EPOR or tEPOR. The chimeric transmembrane receptor transgene may comprise the coding region for a fragment of FKBP and a fragment of either SCF, TPOR, or EGFR. The chimeric transmembrane receptor transgene may also encode a signal peptide. The chimeric transmembrane receptor transgene may also encode a reporter protein such as GFP or YFP.

Because of the dominant nature of the chimeric transmembrane receptor transgene, cleavage by the RNA-guided nuclease at the sgRNA target site can occur at one or both copies of a target locus in a cell. In some embodiments, the cleavage of a target locus will lead to an indel that will result in the expression of chimeric transmembrane receptor transgene in the cell, i.e., under the control of the endogenous target gene promoter. In some embodiments, cleavage of target gene locus in the presence of a donor template leads to integration of a chimeric transmembrane receptor transgene at the target locus, and consequently to the expression of chimeric transmembrane receptor transgene in the cell under the control of the endogenous EPOR promoter. In some embodiments, cleavage of a target sequence in a safe-harbor locus such as CCR5, HBA1, HBB, or EPOR locus in the presence of a donor template leads to integration of a chimeric transmembrane receptor transgene and optionally a therapeutic transgene encoding a protein at the safe harbor locus. In such embodiments, the chimeric transmembrane receptor transgene cDNA and/or therapeutic transgene can be under the control of a heterologous promoter such as SFFV4, PGK1, or UBC. In some embodiments, the integrated chimeric transmembrane receptor transgene cDNA and/or therapeutic transgene is under the control of the endogenous promoter of the safe-harbor locus, e.g., the CCR5, HBA 1, HBB, or EPOR promoter,

sgRNAs

In some embodiments, the single guide RNAs (sgRNAs) of the present disclosure target a safe-harbor locus such as CCR5. HBA1, or HBB or the EPOR locus, sgRNAs interact with a site-directed nuclease such as Cas9 and specifically bind to or hybridize to a target nucleic acid within the genome of a cell, such that the sgRNA and the site-directed nuclease co-localize to the target nucleic acid in the genome of the cell. The sgRNAs as used herein comprise a targeting sequence comprising homology (or complementarity) to a target DNA sequence at, e.g., a HBA1, HBB, or CCR5 locus, and a constant region that mediates binding to Cas9 or another RNA-guided nuclease. The sgRNA can target any sequence within the target gene adjacent to a PAM sequence. The targeted sequence can be within a coding sequence or a non-coding sequence of the gene. In some embodiments, the target sequence comprises one of the sequences shown as SEQ ID NOS: 23-28, or a sequence having, e.g., at least 90%, 91%, 92%, 93%, 94%, 95%, 96%, 97%, 98%, 99%, or more identity to, e.g., comprising 1, 2, 3, or more nucleotide substitutions, additions, or subtractions relative to, one of SEQ ID NOS: 23-28. In embodiments wherein an HBA1 or CCR5 safe-harbor locus is targeted, the guide RNA target sequence comprises the sequence of SEQ ID NO: 23 or SEQ ID NO: 24, respectively, or a sequence having, e.g., at least 90%, 91%, 92%, 93%, 94%, 95%, 96%, 97%, 98%, 99%, or more identity to, e.g., comprising 1, 2, 3, or more nucleotide substitutions, additions or subtractions relative to, SEQ ID NO: 23 or SEQ ID NO: 24. In embodiments wherein an HBB safe-harbor locus is targeted, the guide RNA target sequence comprises the sequence of any one of SEQ ID NOS: 25-27, or a sequence having, e.g., at least 90%, 91%, 92%, 93%, 94%, 95%, 96%, 97%, 98%, 99%, or more identity to, e.g., comprising 1, 2, 3, or more nucleotide substitutions, additions or subtractions relative to any one of SEQ ID NOS: 25-27.

The targeting sequence of the sgRNAs may be, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or 15-25, 18-22, or 19-21 nucleotides in length, and shares homology with a targeted genomic sequence, in particular at a position adjacent to a CRISPR PAM sequence. The sgRNA targeting sequence is designed to be homologous to the target DNA, i.e., to share the same sequence with the non-bound strand of the DNA template or to be complementary to the strand of the template DNA that is bound by the sgRNA. The homology or complementarity of the targeting sequence can be perfect (i.e., sharing 100% homology or 100% complementarity to the target DNA sequence) or the targeting sequence can be substantially homologous (i.e., having less than 100% homology or complementarity, e.g., with 1-4 mismatches with the target DNA sequence).

Each sgRNA also includes a constant region that interacts with or binds to the site-directed nuclease, e.g., Cas9. In the nucleic acid constructs provided herein, the constant region of an sgRNA can be from about 70 to 250 nucleotides in length, or about 75-100 nucleotides in length, 75-85 nucleotides in length, or about 80-90 nucleotides in length, or 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length. The overall length of the sgRNA can be, e.g., from about 80-300 nucleotides in length, or about 80-150 nucleotides in length, or about 80-120 nucleotides in length, or about 90-110 nucleotides in length, or, e.g., 90, 91. 92, 93. 94, 95. 96, 97, 98, 99, 100, 101, 102, 103, 104, 105, 106, 107, 108, 109, or 110 nucleotides in length.

It will be appreciated that it is also possible to use two-piece gRNAs (cr:tracrRNAs) in the present methods, i.e., with separate crRNA and tracrRNA molecules in which the target sequence is defined by the crispr RNA (crRNA), and the tracrRNA provides a binding scaffold for the Cas nuclease.

In some embodiments, the sgRNAs comprise one or more modified nucleotides. For example, the polynucleotide sequences of the sgRNAs may also comprise RNA analogs, derivatives, or combinations thereof. For example, the probes can be modified at the base moiety, at the sugar moiety, or at the phosphate backbone (e.g., phosphorothioates). In some embodiments, the sgRNAs comprise 3′ phosphorothiate internucleotide linkages, 2′-O-methyl-3′-phospholactate modifications, 2′-fluoro-pyrimidines, S-constrained ethyl sugar modifications, or others, at one or more nucleotides. In particular embodiments, the sgRNAs comprise 2′-O-methyl-3′-phosphorothioate (MS) modifications at one or more nucleotides (see, e.g., Hendel et al. (2015) Nat. Biotech. 33(9):985-989, the entire disclosure of which is herein incorporated by reference). In particular embodiments, the 2′-O-methyl-3′-phosphorothioate (MS) modifications are at the three terminal nucleotides of the 5′ and 3′ ends of the sgRNA.

The sgRNAs can be obtained in any of a number of ways. For sgRNAs, primers can be synthesized in the laboratory using an oligo synthesizer. e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

### RNA-Guided Nucleases

Any CRISPR-Cas nuclease can be used in the method, i.e., a CRISPR-Cas nuclease capable of interacting with a guide RNA and cleaving the DNA at the target site as defined by the guide RNA. In some embodiments, the nuclease is Cas9 or Cpf1. In particular embodiments, the nuclease is Cas9. The Cas9 or other nuclease used in the present methods can be from any source, so long that it is capable of binding to an sgRNA as described herein and being guided to and cleaving the specific target (e.g., CCR5, HBA1, HBB, or EPOR) sequence targeted by the targeting sequence of the sgRNA. In particular embodiments, the Cas9 is from Streptococcus pyogenes.

Also disclosed herein are CRISPR/Cas or CRISPR/Cpf1 systems that target and cleave DNA at, e.g., the CCR5, HBA1, or HBB locus. An exemplary CRISPR/Cas system comprises (a) a Cas (e.g.. Cas9) or Cpf1 polypeptide or a nucleic acid encoding said polypeptide, and (b) an sgRNA that hybridizes specifically to CCR5, (or HBA1, HBB, or other safe-harbor locus, or the EPOR locus), or a nucleic acid encoding said guide RNA. In some instances, the nuclease systems described herein further comprise a donor template as described herein. In particular embodiments, the CRISPR/Cas system comprises an RNP comprising an sgRNA targeting CCR5 (or HBA1 or HBB, or other safe harbor locus, or the EPOR locus) and a Cas protein such as Cas9. In some embodiments, the Cas9 is a high fidelity (HiFi) Cas9 (see, e.g., Vakulskas, C. A. et al., Nat. Med. 24, 1216-1224 (2018)).

In addition to the CRISPR/Cas9 platform (which is a type 11 CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9). Streptococcus thermophilus Cas9 (StCas9), Campylobacter jejuni Cas9 (CjCas9) and Neisseria cinerea Cas9 (NcCas9) to name a few. Alternatives to the Cas system include the Francisella novicida Cpf1 (FnCpf1), Acidaminococcus sp. Cpf1 (AsCpf1), and Lachnospiraceae bacterium ND2006 Cpf1 (LbCpf1) systems. Any of the above CRISPR systems may be used to induce a single or double stranded break at the CCR5 locus (or, e.g., the HBA1 or HBB, or other safe harbor locus) to carry out the methods disclosed herein.

Introducing the sgRNA and Cas Protein into Cells

The guide RNA and nuclease can be introduced into the cell using any suitable method, e.g., by introducing one or more polynucleotides encoding the guide RNA and the nuclease into the cell, e.g., using a vector such as a viral vector or delivered as naked DNA or RNA, such that the guide RNA and nuclease are expressed in the cell. In some embodiments, one or more polynucleotides encoding the sgRNA, the nuclease or a combination thereof are included in an expression cassette. In some embodiments, the sgRNA, the nuclease, or both sgRNA and nuclease are expressed in the cell from an expression cassette. In some embodiments, the sgRNA, the nuclease, or both sgRNA and nuclease are expressed in the cell under the control of a heterologous promoter. In some embodiments, one or more polynucleotides encoding the sgRNA and the nuclease are operatively linked to a heterologous promoter. In particular embodiments, the guide RNA and nuclease are assembled into ribonucleoproteins (RNPs) prior to delivery to the cells, and the RNPs are introduced into the cell by, e.g., electroporation. RNPs are complexes of RNA and RNA-binding proteins. In the context of the present methods, the RNPs comprise the RNA-binding nuclease (e.g., Cas9) assembled with the guide RNA (e.g., sgRNA), such that the RNPs are capable of binding to the target DNA (through the gRNA component of the RNP) and cleaving it (via the protein nuclease component of the RNP). As used herein, an RNP for use in the present methods can comprise any of the herein-described guide RNAs and any of the herein-described RNA-guided nucleases.

Animal cells, mammalian cells, preferably human cells, modified ex vivo, in vitro, or in vivo are contemplated. Also included are cells of other primates; mammals, including commercially relevant mammals, such as cattle, pigs, horses, sheep, cats, dogs, mice, rats; birds, including commercially relevant birds such as poultry, chickens, ducks, geese, and/or turkeys.

In some embodiments, the cell is an embryonic stem cell, a stem cell, a progenitor cell, a pluripotent stem cell, an induced pluripotent stem (iPS) cell, a somatic stem cell, a differentiated cell, a mesenchymal stem cell or a mesenchymal stromal cell, a neural stem cell, a hematopoietic stem cell or a hematopoietic progenitor cell, an adipose stem cell, a keratinocyte, a skeletal stem cell, a muscle stem cell, a fibroblast, an NK cell, a B-cell, a T cell, or a peripheral blood mononuclear cell (PBMC). In particular embodiments, the cells are CD34⁺ hematopoietic stem and progenitor cells (HSPCs), e.g., cord blood-derived (CB), adult peripheral blood-derived (PB), or bone marrow derived HSPCs.

HSPCs can be isolated from a subject, e.g., by collecting mobilized peripheral blood and then enriching the HSPCs using the CD34 marker. In some embodiments, the cells are from a subject with a genetic condition involving erythroid cells (e.g., alpha-thalassemia, beta-thalassemia, sickle cell disease), or from a subject with a condition that could be treated with genetically modified HSPCs expressing a beneficial and/or therapeutic protein (e.g., hemophilia B, phenylketonuria, mucopolysaceharidosis type 1. Gaucher disease, Krabbe disease). In some embodiments, a method is provided of treating a subject with any of the herein-described conditions or disorders (e.g., alpha-thalassemia, beta-thalassemia, sickle cell disease, hemophilia B, phenylketonuria, Gaucher disease, Krabbe disease) comprising genetically modifying a plurality of HSPCs isolated from the subject so as to integrate a therapeutic transgene for the particular condition or disorder (e.g., a transgene encoding α-globin, β-globin, factor IX, phenylalanine hydroxylase (PAH), iduronidase, glucocerebrosidase, galactocerebrosidase, and the like), and also to effect the expression of a chimeric transmembrane receptor transgene as described herein, and reintroducing the HSPCs into the subject. In certain embodiments, the therapeutic transgene is a full-length (e.g., from start codon to stop codon, including introns) transgene comprising a corrective (e.g., wild-type) sequence of an endogenous gene containing one or more deleterious mutations in the HSPCs or encoding a protein that is deficient in the HSPCs. In particular embodiments, HSPCs expressing a chimeric transmembrane receptor transgene and comprising a therapeutic transgene or other beneficial genetic modification proliferate more rapidly in vivo and become enriched relative to equivalent HSPCs not expressing a chimeric transmembrane receptor transgene, e.g., in cells from the subject that have not been genetically modified using the present methods.

To avoid immune rejection of the modified cells when administered to a subject, the cells to be modified are preferably derived from the subject's own cells. Thus, preferably the mammalian cells are antologous cells from the subject to be treated with the modified cells. In some embodiments, however, the cells are allogeneic, i.e., isolated from an HLA-matched or HLA-compatible, or otherwise suitable, donor.

In some embodiments, cells are harvested from the subject and modified according to the methods disclosed herein, which can include selecting certain cell types, optionally expanding the cells and optionally culturing the cells, and which can additionally include selecting cells that contain a chimeric transmembrane receptor transgene integrated into the CCR5 (or HBA1 or HBB, or other safe harbor) locus, or the EPOR locus, and/or cells that have been modified to express a therapeutic or otherwise beneficial transgene. In particular embodiments, such modified cells are then reintroduced into the subject.

Further disclosed herein are methods of using said nuclease systems to produce the modified host cells described herein, comprising introducing into the cell (a) an RNP of the present disclosure that targets and cleaves DNA at the CCR5 (or HBA1, or HBB, or other safe harbor) locus, or the EPOR locus, and optionally (b) a homologous donor template or vector as described herein. Each component can be introduced into the cell directly or can be expressed in the cell by introducing a nucleic acid encoding the components of said one or more nuclease systems.

In some aspects, the present methods target integration of a chimeric transmembrane receptor transgene, i.e., at a safe harbor locus such as CCR5, HBA1, or HBB, or EPOR, in a host cell ex vivo. In some embodiments, the present methods can comprise (a) introducing a donor template comprising a therapeutic transgene encoding a protein and the chimeric transmembrane receptor transgene at a safe harbor locus in the genome of the cell, e.g., to introduce a therapeutic genetic modification (such as the introduction of an HBA1, HBA 2, HBB, PDGFB, FIX, LDLR, PAH, IDUA, GBA, or GALC transgene or vector into the cell at the safe harbor locus), optionally after expanding said cells, or optionally before expanding said cells, and (b) optionally culturing the cell. In particular embodiments, the first and second homology regions of the donor template flank both the therapeutic transgene and the chimeric transmembrane receptor transgene. In certain embodiments, the donor template is a bicistronic cassette comprising an internal ribosome entry site (IRES) between the therapeutic transgene and the chimeric transmembrane receptor transgene. An exemplary IRES sequence is shown as SEQ ID NO: 29. In certain other embodiments, the donor template is a bicistronic cassette comprising a nucleic acid sequence encoding a 2A cleavage peptide (e.g., a member of the 2A peptide family such as a T2A, P2A, E2A, or F2A cleavage peptide) between the therapeutic transgene and the chimeric transmembrane receptor transgene. Exemplary nucleic acid sequences encoding T2A and P2A cleavage peptides are shown as SEQ ID NOS: 30 and 31, respectively. In some instances, the 2A cleavage peptide is a T2A or P2A cleavage peptide. In other instances, the 2A cleavage peptide is a peptide having sequence similarity and functional interchangeability to a T2A or P2A cleavage peptide, such as an E2A or F2A cleavage peptide. In some instances, the therapeutic transgene is 5′ of the IRES sequence or the sequence encoding the 2A cleavage peptide and the chimeric transmembrane receptor transgene is 3′ of the IRES sequence or the sequence encoding the 2A cleavage peptide. In some such instances, the first homology region is 5′ of the therapeutic transgene and the second homology region is 3′ of the chimeric transmembrane receptor transgene. In other instances, the chimeric transgene is 5′ of the IRES sequence or the sequence encoding the 2A cleavage peptide and the therapeutic transgene is 3′ of the IRES sequence or the sequence encoding the 2A cleavage peptide. In some such instances, the first homology region is 5′ of the chimeric transmembrane receptor transgene and the second homology region is 3′ of the therapeutic transgene. In other embodiments, the present methods can further comprise (a) introducing a second guide RNA and donor template into the cell, e.g., to introduce a second, therapeutic genetic modification (such as the introduction of an HBA1, HBA 2, HBB, PDGFB, FIX, LDLR, PAH, IDUA, GBA, or GALC transgene or vector into the cell at a second genomic locus), optionally after expanding said cells, or optionally before expanding said cells, and (b) optionally culturing the cell.

In some embodiments, the disclosure herein contemplates a method of producing a modified mammalian host cell, the method comprising introducing into a mammalian cell: (a) an RNP comprising a Cas nuclease such as Cas9 and an sgRNA as described herein, and optionally (b) a homologous donor template or vector as described herein.

In any of these methods, the nuclease can produce one or more single stranded breaks within the targeted (e.g., CCR5, HBA1, or HBB) locus, or the EPOR locus, or a double-stranded break within the targeted locus. In these methods, the targeted locus is modified by homologous recombination with a donor template or vector to result in insertion of the transgene into the locus. The methods can further comprise (c) selecting cells that contain the integrated transgene at the targeted locus.

In some embodiments, i53 (Canny et al. (2018) Nat Biotechnol 36:95) is introduced into the cell in order to promote integration of the donor template by homology directed repair (HDR) versus integration by non-homologous end-joining (NHEJ). For example, an mRNA encoding i53 can be introduced into the cell, e.g., by electroporation at the same time as an sgRNA-Cas9 RNP. The sequence of i53 can be found, inter alia, at www.addgcne.org/92170/sequences/.

Techniques for the insertion of transgenes, including large transgenes, capable of expressing functional proteins, including enzymes, cytokines, antibodies, and cell surface receptors are known in the art (See, e.g.. Bak and Porteus, Cell Rep. 2017 Jul. 18; 20(3): 750-756 (integration of EGFR); Kanojia et al., Stem Cells. 2015 Oct:33(10):2985-94 (expression of anti-Her2 antibody); Eyquem et al., Nature. 2017 Mar. 2; 543(7643):113-117 (site-specific integration of a CAR); O'Connell et al., 2010 PLoS ONE 5(8): e12009 (expression of human IL-7); Tuszynski et al., Nat Med. 2005 May; 11(5):551-5 (expression of NGF in fibroblasts); Sessa et al., Lancet. 2016 Jul. 30; 388(10043):476-87 (expression of arylsulfatase A in ex vivo gene therapy to treat MLD): Rocca et al., Science Translational Medicine 25 Oct. 2017: Vol. 9, Issue 413, eaaj2347 (expression of frataxin); Bak and Porteus, Cell Reports, Vol. 20, Issue 3, 18 Jul. 2017, Pages 750-756 (integrating large transgene cassettes into a single locus), Dever et al., Nature 17 Nov. 2016: 539, 384-389 (adding tNGFR into hematopoietic stem cells (HSC) and HSPCs to select and enrich for modified cells); each of which is herein incorporated by reference in its entirety.

### Homologous Repair Templates

The transgene to be integrated, which is comprised by a polynucleotide or donor construct, can be any chimeric transmembrane receptor transgene whose gene product can provide chimeric transmembrane receptor expression in red blood cells or other cells of the erythroid lineage, and particularly provide inducible differentiation and/or drive the elevated proliferation of the modified cells relative to equivalent cells lacking the chimeric transmembrane receptor. For example, the transgene could be integrated at the EPOR locus. Alternatively, the transgene could be integrated a genomic location outside of the EPOR locus. In some instances, the transgene could be integrated using an EPOR left homology arm comprising SEQ ID NO: 18. In some instances, the transgene could be integrated using an EPOR right homology arm comprising SEQ ID NO: 19. For example, in some embodiments, a chimeric transmembrane receptor transgene and optionally a therapeutic transgene encoding a protein is integrated at a safe harbor locus such as CCR5. HBA1, or HBB.

In some embodiments, the donor template further comprises a therapeutic transgene. An exemplary, non-limiting list of suitable transgenes includes HBA/(hemoglobin subunit alpha 1; see, e.g., NCBI Gene ID No. 3039), HBA2 (hemoglobin subunit alpha 2; see, e.g., NCBI Gene ID No. 3040), HBB (hemoglobin subunit beta; see, e.g., NCBI Gene ID No. 3043), PDFGB (platelet-derived growth factor subunit B; see, e.g., NCBI Gene ID No. 5155), IDUA (alpha-L-iduronidase; see, e.g., NCBI Gene ID No. 3425), PAH (phenylalanine hydroxylase; see, e.g., NCBI Gene ID No. 5053), Factor IX (or FIX; see, e.g., NCBI Gene ID NO. 2158), including Hyperactive Factor IX Padua, or the Padua Variant (see. e.g., Simioni et al., (2009) NEJM 361:1671-1675; Cantore et al. (2012) Blood 120:4517-4520; Monahan et al., (2015) Hum. Gene. Ther. 26:69-81), LDLR (low density lipoprotein receptor; see, e.g., NCBI Gene ID No. 3949), and others. In particular embodiments, the first and second homology anns of the donor template flank both the therapeutic transgene and the chimeric transmembrane receptor transgene. In certain embodiments, the donor template is a bicistronic cassette comprising an internal ribosome entry site (IRES) between the therapeutic transgene and the chimeric transmembrane receptor transgene. In certain other embodiments, the donor template is a bicistronic cassette comprising a nucleic acid sequence encoding a 2A cleavage peptide (e.g., a member of the 2A peptide family such as a T2A, P2A, E2A, or F2A cleavage peptide) between the therapeutic transgene and the chimeric transmembrane receptor transgene. In some instances, the therapeutic transgene is 5′ of the IRES sequence or the sequence encoding the 2A cleavage peptide and the chimeric transmembrane receptor transgene is 3 of the IRES sequence or the sequence encoding the 2A cleavage peptide. In some such instances, the first homology arm is 5′ of the therapeutic transgene and the second homology arm is 3′ of the chimeric transmembrane receptor transgene. In other instances, the chimeric transmembrane receptor transgene is 5′ of the IRES sequence or the sequence encoding the 2A cleavage peptide and the therapeutic transgene is 3′ of the IRES sequence or the sequence encoding the 2A cleavage peptide. In some such instances, the first homology arm is 5′ of the chimeric transmembrane receptor transgene and the second homology arm is 3′ of the therapeutic transgene. In some instances, the IRES sequence comprises SEQ ID NO: 29. In some instances, the T2A cleavage peptide coding sequence comprises SEQ ID NO: 30. In some instance, the P2A cleavage peptide coding sequence comprises SEQ ID NO: 31. In some instances, the bicistronic construct encodes an HBB polypeptide comprising SEQ ID NO: 6. In some instances, the bicistronic construct encodes an iEPOR polypeptide comprising SEQ ID NO: 7.

In other embodiments, a second donor template is used that comprises a therapeutic transgene, e.g., an HBA1, HBA2, HBB, PDGFB, IDUA, GBA, FIX, LDLR, PAH, or GALC transgene.

In some embodiments, expression of a chimeric transmembrane receptor transgene, and/or a second transgene such as an HBA1, HBA2, HBB, IDUA, PDGFB, GBA, FIX, LDLR, PAH, or GALC transgene, is driven by a heterologous promoter such as HBA1, HBA2, HBB, PGK1, SFFV, or UBC. In other embodiments, a chimeric transmembrane receptor transgene, and/or a second transgene such as an HBA1, HBA 2, HBB, IDUA, PDGFB, GBA, FIX, LDLR, PAH, or GALC transgene, is driven by an endogenous promoter such as HBA1, HBA2, or HBB,

In some embodiments, the transgene in the homologous repair template is codon-optimized, e.g., comprises at least about 70%, 75%, 80%, 85%, 90%, 95%, or more homology to the corresponding wild-type coding sequence or cDNA, or a fragment thereof such as in the case of a truncated EPOR.

A transgene as used herein may also comprise optional elements such as introns, WPREs, polyA regions, UTRs (e.g., 5′ or 3′ UTRs). In particular embodiments, the template comprises a polyA sequence or signal, e.g., a bovine growth hormone polyA sequence or a rabbit beta-globin polyA sequence, at the 3′ end of the cDNA. In particular embodiments, a Woodchuck Hepatitis Virus Posttranscriptional Regulatory Element (WPRE) is included within the 3′UTR of the template, e.g., between the 3′ end of the coding sequence and the 5′ end of the polyA sequence, so as to increase the expression of the transgene. Any suitable WPRE sequence can be used; See, e.g., Zufferey et al. (1999) J. Virol. 73(4):2886-2892: Donello, et al. (1998). J Virol 72: 5085-5092; Loeb, et al. (1999). Hum Gene Ther 10: 2295-2305; the entire disclosures of which are herein incorporated by reference).

To facilitate homologous recombination, the transgene is flanked within the polynucleotide or donor construct by sequences homologous to the target genomic sequence. In particular embodiments, the transgene is flanked by one sequence homologous to the region 5′ to the cleavage site (e.g., starting at or around the guide RNA target sequence and running upstream) and a second sequence homologous to the region 3′ of the site of cleavage (e.g., starting at or around the guide RNA target site and running downstream).In some embodiments wherein the HBA1 safe harbor locus is targeted, the donor template comprises a left homology sequence comprising the sequence shown as SEQ ID NO: 12 or a subsequence thereof, or to a sequence comprising at least about 70%, 75%, 80%, 85%, 90%, 95%, 96%, 97%, 98%, 99% or more homology to SEQ ID NO: 12 or a subsequence thereof. In some embodiments wherein the HBA1 safe harbor locus is targeted, the donor template comprises a right homology sequence comprising the sequence shown as SEQ ID NO: 13 or a subsequence thereof, or to a sequence comprising at least about 70%, 75%, 80%, 85%, 90%, 95%, 96%, 97%, 98%, 99% or more homology to SEQ ID NO: 13 or a subsequence thereof. In some embodiments wherein the CCR5 safe harbor locus is targeted, the donor template comprises a left homology sequence comprising the sequence shown as SEQ ID NO: 14 or a subsequence thereof, or to a sequence comprising at least about 70%, 75%, 80%, 85%, 90%, 95%, 96%, 970, 98%, 99% or more homology to SEQ ID NO: 14 or a subsequence thereof. In some embodiments wherein the CCR5 safe harbor locus is targeted, the donor template comprises a right homology sequence comprising the sequence shown as SEQ ID NO: 15 or a subsequence thereof, or to a sequence comprising at least about 70%, 75%, 80%, 85%, 90%, 950, 96%, 97%, 98%, 99% or more homology to SEQ ID NO: 15 or a subsequence thereof. In some embodiments wherein the HBB safe harbor locus is targeted, the donor template comprises a left homology sequence comprising the sequence shown as SEQ ID NO: 16 or a subsequence thereof, or to a sequence comprising at least about 70%, 75%, 80%, 85%, 90%, 95%, 96%, 97%, 98%, 99% or more homology to SEQ ID NO: 16 or a subsequence thereof In some embodiments wherein the HBB safe harbor locus is targeted, the donor template comprises a right homology sequence comprising the sequence shown as SEQ ID NO: 17 or a subsequence thereof, or to a sequence comprising at least about 70%, 75%, 80%, 85%, 90%, 95%, 96%, 97%, 98%, 99% or more homology to SEQ ID NO: 17 or a subsequence thereof.

In some embodiments, a chimeric transmembrane receptor transgene (and optionally a therapeutic transgene such as HBA1, HBA2, HBB, PDGFB, FIX, LDLR, PAH, IDUA, GBA, or GALC) replaces all or part of a safe harbor gene such as CCR5, HBA1, or HBB such that its expression is driven by the endogenous CCR5, HBA1, or HBB promoter. In some embodiments, a chimeric transmembrane receptor transgene (and optionally a therapeutic transgene such as HBA1, HBA2, HBB, PDGFB, FIX, LDLR, PAH, IDUA, GBA, or GALC) is integrated into a safe harbor locus such as CCR5, HBA1, or HBB wherein the expression of the transgene is driven by a heterologous promoter such as HBA1, HBA2, HBB, PGK1, SFFV, or UBC. In some embodiments, a part or a fragment of the target gene is replaced by the transgene. In some embodiments, the whole coding sequence of the target gene is replaced by the transgene. In some embodiments, the coding sequence and regulatory sequences of the transgene is replaced by the transgene. In some embodiments, the target gene sequence replaced by the transgene comprises an open reading frame. In some embodiments, the target gene sequence replaced by the transgene comprises an expression cassette. In some embodiments, the target gene sequence replaced by the transgene comprises a sequence that transcribes into a precursor mRNA. In some embodiments, the target gene sequence replaced by the transgene comprises a 5′ UTR, one or more introns, one or more exons, and a 3′ UTR.

In some embodiments, the 5′ (or left) homology arm is at least 100 bp, 200 bp, 300 bp. 400 bp, 500 bp, 600 bp, 700 bp, 800 bp, 900 bp, 1000 bp or more in length. In some embodiments, the 5′ homology arm is 100 bp, 150 bp, 200 bp, 250 bp, 275 bp, 300 bp, 325 bp, 350 bp, 375 bp, 400 bp, 450 bp, or greater than 500 bp in length. In some embodiments, the 5′ homology arm is at least 400 bp in length. In some embodiments, the 5′ homology arm is at least 500 bp, 600 bp, 700 bp, 800 bp, 900 bp, or 1000 bp in length. In some embodiments, the 5′ homology arm is at least 850 bp in length. In some embodiments, the 5′ homology arm is 400-500 bp. In some embodiments, the 5′ homology arm is 400-500 bp, 400-550 bp, 400-600 bp, 400-650 bp, 400-700 bp, 400-750 bp, 400-800 bp, 400-850 bp, 400-900 bp, 400-950 bp, 400-1000 bp, 400-1100 bp, 400-1200 bp, 400-1300 bp, 400-1400 bp, 450-500 bp, 450-550 bp, 450-600 bp, 450-650 bp, 450-700 bp, 450-750 bp, 450-800 bp, 450-850 bp, 450-900 bp, 450-950 bp, 450-1000 bp, 450-1100 bp, 450-1200 bp, 450-1300 bp, 450-1450 bp, 500-600 bp, 500-650 bp, 500-700 bp, 500-750 bp, 500-800 bp, 500-850 bp, 500-900 bp, 500-950 bp, 500-1000 bp, 500-1100 bp, 500-1200 bp, 500-1300 bp, 500-1500 bp, 550-600 bp, 550-650 bp, 550-700 bp, 550-750 bp, 550-800 bp, 550-850 bp, 550-900 bp, 550-950 bp, 550-1000 bp, 550-1100 bp, 550-1200 bp, 550-1300 bp, 550-1500 bp. 600-650 bp, 600-700 bp, 600-750 bp, 600-800 bp, 600-850 bp, 600-900 bp, 600-950 bp, 600-1000 bp, 600-1100 bp, 600-1200 bp, 600-1300 bp, 600-1600 bp, 650-700 bp, 650-750 bp, 650-800 bp, 650-850 bp, 650-900 bp, 650-950 bp, 650-1000 bp, 650-1100 bp, 650-1200 bp, 650-1300 bp, 650-1500 bp, 700-700 bp, 700-750 bp, 700-800 bp, 700-850 bp, 700-900 bp, 700-950 bp, 700-1000 bp, 700-1100 bp, 700-1200 bp, 700-1300 bp, 700-1500 bp, 750-800 bp, 750-850 bp, 750-900 bp, 750-950 bp, 750-1000 bp, 750-1100 bp, 750-1200 bp, 750-1300 bp, 750-1500 bp, 800-850 bp, 800-900 bp, 800-950 bp, 800-1000 bp, 800-1100 bp, 800-1200 bp, 800-1300 bp, 800-1500 bp, 850-900 bp, 850-950 bp, 850-1000 bp, 850-1100 bp, 850-1200 bp, 850-1300 bp, 850-1500 bp, 900-950 bp, 900-1000 bp, 900-1100 bp, 900-1200 bp, 900-1300 bp, 900-1500 bp, 1000-1100 bp, 1100-1200 bp, 1200-1300 bp, 1300-1400 bp, or 1400-1500 bp in length. In particular embodiments, the 5′ homology arm is about 900 nucleotides in length.

In some embodiments, the 3′ (or right)homology arm is at least 100 bp, 200 bp, 300 bp. 400 bp, 500 bp, 600 bp, 700 bp, 800 bp, 900 bp, 1000 bp or more in length. In some embodiments, the 3′ homology arm is 100 bp, 150 bp, 200 bp, 250 bp, 275 bp, 300 bp, 325 bp, 350 bp, 375 bp, 400 bp, 450 bp, or greater than 500 bp in length. In some embodiments, the 3′ homology arm is at least 400 bp in length. In some embodiments, the 3′ homology arm is at least 500 bp, 600 bp, 700 bp, 800 bp, 900 bp, or 1000 bp in length. In some embodiments, the 3′ homology arm is at least 850 bp in length. In some embodiments, the 3′ homology arm is 400-500 bp. In some embodiments, the 3′ homology arm is 400-500 bp, 400-550 bp, 400-600 bp, 400-650 bp, 400-700 bp, 400-750 bp, 400-800 bp, 400-850 bp, 400-900 bp, 400-950 bp, 400-1000 bp, 400-1100 bp, 400-1200 bp, 400-1300 bp, 400-1400 bp, 450-500 bp, 450-550 bp, 450-600 bp, 450-650 bp, 450-700 bp, 450-750 bp, 450-800 bp, 450-850 bp, 450-900 bp, 450-950 bp, 450-1000 bp, 450-1100 bp, 450-1200 bp, 450-1300 bp, 450-1450 bp, 500-600 bp, 500-650 bp, 500-700 bp, 500-750 bp, 500-800 bp, 500-850 bp, 500-900 bp, 500-950 bp, 500-1000 bp, 500-1100 bp, 500-1200 bp, 500-1300 bp, 500-1500 bp, 550-600 bp, 550-650 bp, 550-700 bp, 550-750 bp, 550-800 bp, 550-850 bp, 550-900 bp, 550-950 bp, 550-1000 bp, 550-1100 bp, 550-1200 bp, 550-1300 bp, 550-1500 bp. 600-650 bp, 600-700 bp, 600-750 bp, 600-800 bp, 600-850 bp, 600-900 bp, 600-950 bp, 600-1000 bp, 600-1100 bp, 600-1200 bp, 600-1300 bp, 600-1600 bp, 650-700 bp, 650-750 bp, 650-800 bp, 650-850 bp, 650-900 bp, 650-950 bp, 650-1000 bp, 650-1100 bp, 650-1200 bp, 650-1300 bp, 650-1500 bp, 700-700 bp, 700-750 bp, 700-800 bp, 700-850 bp, 700-900 bp, 700-950 bp, 700-1000 bp, 700-1100 bp, 700-1200 bp, 700-1300 bp, 700-1500 bp, 750-800 bp, 750-850 bp, 750-900 bp, 750-950 bp, 750-1000 bp, 750-1100 bp, 750-1200 bp, 750-1300 bp, 750-1500 bp, 800-850 bp, 800-900 bp, 800-950 bp, 800-1000 bp, 800-1100 bp, 800-1200 bp, 800-1300 bp, 800-1500 bp, 850-900 bp, 850-950 bp, 850-1000 bp, 850-1100 bp, 850-1200 bp, 850-1300 bp, 850-1500 bp, 900-950 bp, 900-1000 bp, 900-1100 bp, 900-1200 bp, 900-1300 bp, 900-1500 bp, 1000-1100 bp, 1100-1200 bp, 1200-1300 bp, 1300-1400 bp, or 1400-1500 bp in length. In particular embodiments, the 3′ homology arm is about 900 nucleotides in length.

The provided chimeric transmembrane receptor polypeptide can optionally include one or more linker peptide sequences. In some embodiments, the chimeric transmembrane receptor polypeptide includes two linker peptide sequences. In some embodiments, the receptor polypeptide includes more than two linker peptide sequences. In particular embodiments, the polynucleotide is introduced using a recombinant adeno-associated viral vector (rAAV). For example, the rAAV can be from serotype 1 (e.g., an rAAV1 vector), 2 (e.g., an rAAV2 vector), 3 (e.g., an rAAV3 vector), 4 (e.g., an rAAV4 vector), 5 (e.g., an rAAV5 vector), 6 (e.g., an rAAV6 vector), 7 (e.g., an rAAV7 vector), 8 (e.g., an rAAV8 vector), 9 (e.g., an rAAV9 vector), 10 (e.g., an rAAV10 vector), or 11 (e.g., an rAAV11 vector). In particular embodiments, the vector is an rAAV6 vector. In some instances, the donor template is single stranded, double stranded, a plasmid or a DNA fragment. In some instances, plasmids comprise elements necessary for replication, including a promoter and optionally a 3′ UTR.

Further disclosed herein are vectors comprising (a) one or more nucleotide sequences homologous to the EPOR locus, and (b) a chimeric transmembrane receptor transgene as described herein. The vector can be a viral vector, such as a retroviral, lentiviral (both integration competent and integration defective lentiviral vectors), adenoviral, adeno-associated viral or herpes simplex viral vector. Viral vectors may further comprise genes necessary for replication of the viral vector.

In some embodiments, the targeting construct comprises. (1) a viral vector backbone. e.g., an AAV backbone, to generate virus; (2) arms of homology to the target site of at least 200 bp but ideally at least 400 bp or at least 900 on each side to assure high levels of reproducible targeting to the site (see, Porteus, Annual Review of Pharmacology and Toxicology, Vol. 56:163-190 (2016); which is hereby incorporated by reference in its entirety); (3) a transgene encoding a functional alpha globin protein and capable of expressing the functional alpha globin protein, a polyA sequence, and optionally a WPRE element; and optionally (4) an additional marker gene to allow for enrichment and/or monitoring of the modified host cells. Any AAV known in the art can be used. In some embodiments the primary AAV serotype is AAV6. In some embodiments, the vector, e.g., rAAV6 vector, comprising the donor template is from about 1-2 kb, 2-3 kb. 3-4 kb, 4-5 kb, 5-6 kb, 6-7 kb, 7-8 kb, or larger.

Suitable marker genes are known in the art and include Myc, HA, FLAG, GFP, YFP, truncated NGFR, truncated EGFR, truncated CD20, truncated CD19, as well as antibiotic resistance genes. In some embodiments, the homologous repair template and/or vector (e.g., AAV6) comprises an expression cassette comprising a coding sequence for truncated nerve growth factor receptor (tNGFR), operably linked to a promoter such as the Ubiquitin C promoter.

The inserted construct can also include other safety switches, such as a standard suicide gene into the locus (e.g., iCasp9) in circumstances where rapid removal of cells might be required due to acute toxicity. The present disclosure provides a robust safety switch so that any engineered cell transplanted into a body can be eliminated, e.g., by removal of an auxotrophic factor. This is especially important if the engineered cell has transformed into a cancerous cell.

In some embodiments, the present methods allow for the insertion of the donor template in at least about 5%, 10%, 15%, 20%, 25%, 30%, 35%, 40%, 45%, 50%, 55%, 60%, 65%, 70%, 75%, 80%, 85%, 90%, 95%, or more cells, e.g., HSPCs from a subject.

In some embodiments, the CRISPR-mediated systems as described herein (e.g., comprising a guide RNA, RNA-guided nuclease, and optionally homologous repair template) are assessed in primary HSPCs, e.g., as derived from mobilized peripheral blood or from cord blood. In such embodiments, the HSPCs can be WT primary HSPCs (e.g., for initial testing of the system) or from patient-derived HSPCs (e.g., for pre-clinical in vitro testing). In some embodiments, the HSPCs are cultured in vitro and allowed to differentiate into RBCs to confirm the elevated rate of proliferation relative to unmodified cells (as measured, e.g., by a co-culture experiment in the presence or absence of EPO as described in Example 1 or by other methods of determining proliferation rate such as BrdU incorporation or by monitoring the number of cells in a culture over time) prior to the reintroduction of HSPCs into a subject.

### 6. Methods of treatment

Following the integration of the chimeric transmembrane receptor polypeptide into the genome of the HSPC and, in particular embodiments, of a therapeutic transgene as described herein, and optionally following confirmation of expression of the encoded therapeutic protein and/or of the elevated proliferation rate of the cells, a plurality of modified HSPCs can be reintroduced into the subject. In one embodiment, the HSPCs are introduced by intrafemoral injection, such that they can populate the bone marrow and differentiate into, e.g., red blood cells. In some embodiments, the HSPCs are introduced by intravenous injection. In some embodiments, the HSPCs are induced to initiate differentiation into red blood cells in vitro, and the modified erythroid lineage cells are then re-introduced into the subject.

Disclosed herein, in some embodiments, are methods of treating a genetic condition or disorder (e.g., alpha-thalassemia, beta-thalassemia, sickle cell disease, hemophilia B, phenylketonuria, mucopolysaccharidosis type 1, Gaucher disease, Krabbe disease, and the like) in an individual in need thereof, the method comprising genetically modifying HSPCs from the individual so as to provide a beneficial effect (e.g., by introducing a therapeutic transgene for correcting a mutation underlying the condition or disorder, or for providing to the individual a protein replacement therapy) and also such that they express chimeric transmembrane receptor transgene and are therefore enriched in vivo following reintroduction of the cells to the individual.

The present methods allow for the efficient integration of a donor template comprising a therapeutic transgene and a chimeric transmembrane receptor transgene at a safe harbor locus. In some embodiments, expression of the therapeutic transgene and the chimeric transmembrane receptor transgene causes an enrichment of genetically modified HSPCs in a population of HSPCs, e.g., over the course of red blood cell differentiation, as compared to expression of the therapeutic transgene in the absence of expression of the chimeric transmembrane receptor transgene. In certain embodiments, the present methods allow for the insertion of the donor template in at least about 5%, 10%, 15%, 20%, 25%, 30%, 35%, 40%, 45%, 50%, 55%, 60%, 65%, 70%, 75%, 80%, 85%, 90%, 95%, or more HSPCs in a population of HSPCs, e.g., over the course of red blood cell differentiation. In certain embodiments, expression of the therapeutic transgene and the chimeric transmembrane receptor transgene increases the proportion of genetically modified HSPCs in a population of HSPCs by at least about 5%, 10%, 15%, 20%, 25%, 30%, 35%, 40%, 45%, 50%, 55%, 60%, 65%, 70%, 75%, 80%, 85%, 90%, 95%, or more, e.g., over the course of red blood cell differentiation, as compared to expression of the therapeutic transgene in the absence of expression of the chimeric transmembrane receptor transgene. In some embodiments, expression of the therapeutic transgene and the chimeric transmembrane receptor transgene increases a level of adult hemoglobin tetramers in the genetically modified HSPCs by at least about 5%, 10%, 15%, 20%, 25%, 30%, 35%, 40%, 45%, 50%, 55%, 60%, 65%, 70%, 75%, 80%, 85%, 90%, 95%, or more as compared to expression of the therapeutic transgene in the absence of expression of the chimeric transmembrane receptor transgene.

### Pharmaceutical Compositions

Disclosed herein, in some embodiments, are methods, compositions and kits for use of the modified cells, including pharmaceutical compositions, therapeutic methods, and methods of administration. Although the descriptions of pharmaceutical compositions provided herein are principally directed to pharmaceutical compositions which are suitable for administration to humans, it will be understood by the skilled artisan that such compositions are generally suitable for administration to any animals.

In some embodiments, a pharmaceutical composition comprising a modified autologous host cell as described herein is provided. The modified autologous host cell is genetically engineered to comprise an integrated chimeric transmembrane receptor transgene at a safe harbor locus such as CCR5, HBA1, or HBB, or the EPOR locus, as well as optionally a second, therapeutic genetic modification as described herein (e.g., a therapeutic transgene integrated at the safe harbor locus). The modified host cell of the disclosure herein may be formulated using one or more excipients to, e.g.: (1) increase stability: (2) alter the biodistribution (e.g., target the cell line to specific tissues or cell types); (3) alter the release profile of an encoded therapeutic factor.

Formulations of the present disclosure can include, without limitation, saline, liposomes, lipid nanoparticles, polymers, peptides, proteins, and combinations thereof. Formulations of the pharmaceutical compositions described herein may be prepared by any method known or hereafter developed in the art of pharmacology. As used herein the term “pharmaceutical composition” refers to compositions including at least one active ingredient (e.g., a modified host cell) and optionally one or more pharmaceutically acceptable excipients. Pharmaceutical compositions of the present disclosure may be sterile.

Relative amounts of the active ingredient (e.g., the modified host cell), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, or at least 80% (w/w) active ingredient.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006: incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

### Dosing and Administration

The modified host cells of the present disclosure included in the pharmaceutical compositions described above may be administered by any delivery route, systemic delivery or local delivery, which results in a therapeutically effective outcome. These include, but are not limited to, enteral, gastroenteral, epidural, oral, transdermal, intracerbral, intracerebroventricular, epicutaneous, intradermal, subcutaneous, nasal, intravenous, intra-arterial, intramuscular, intracardiac, intraosseous, intrathecal, intraparenchymal, intraperitoneal, intravesical, intravitreal, intracavemous), interstitial, intra-abdominal, intralymphatic, intramedullary, intrapulmonary, intraspinal, intrasynovial, intrathecal, intratubular, parenteral, percutaneous, periarticular, peridural, perineural, periodontal, rectal, soft tissue, and topical.

In some embodiments, a subject undergoes a conditioning regime before cell transplantation. For example, before hematopoietic stem cell transplantation, a subject may undergo myeloablative therapy, non-myeloablative therapy or reduced intensity conditioning to prevent rejection of the stem cell transplant even if the stem cell originated from the same subject. The conditioning regime may involve administration of cytotoxic agents. The conditioning regime may also include immunosuppression, antibodies, and irradiation. Other possible conditioning regimens include antibody-mediated conditioning (see, e.g., Czechowicz et al., 318(5854) Science 1296-9 (2007); Palchaudari et al., 34(7) Nature Biotechnology 738-745 (2016); Chhabra et al., 10:8(351) Science Translational Medicine 351ra105 (2016)) and CAR T-mediated conditioning (see, e.g.. Arai et al., 26(5) Molecular Therapy 1181-1197 (2018); each of which is hereby incorporated by reference in its entirety). For example, conditioning needs to be used to create space in the brain for microglia derived from engineered hematopoietic stem cells (HSCs) to migrate in to deliver the protein of interest (as in recent gene therapy trials for ALD and MLD). The conditioning regimen is also designed to create niche “space” to allow the transplanted cells to have a place in the body to engraft and proliferate. In HSPC transplantation, for example, the conditioning regimen creates niche space in the bone marrow for the transplanted HSPCs to engraft. Without a conditioning regimen, the transplanted HSCs cannot engraft.

Certain aspects of the present disclosure are directed to methods of providing pharmaceutical compositions including the modified host cell of the present disclosure to target tissues of mammalian subjects, by contacting target tissues with pharmaceutical compositions including the modified host cell under conditions such that they are substantially retained in such target tissues. In some embodiments, pharmaceutical compositions including the modified host cell include one or more cell penetration agents, although “naked” formulations (such as without cell penetration agents or other agents) are also contemplated, with or without pharmaceutically acceptable excipients.

The present disclosure additionally provides methods of administering modified host cells in accordance with the disclosure to a subject in need thereof. The pharmaceutical compositions including the modified host cell, and compositions of the present disclosure may be administered to a subject using any amount and any route of administration effective for preventing, treating, or managing the condition or disorder, e.g., alpha-thalassemia, beta-thalassemia, sickle cell disease, etc. The exact amount required will vary from subject to subject, depending on the species, age, and general condition of the subject, the severity of the disease, the particular composition, its mode of administration, its mode of activity, and the like. The subject may be a human, a mammal, or an animal. The specific therapeutically or prophylactically effective dose level for any particular individual will depend upon a variety of factors including the condition or disorder being treated and the severity of the condition or disorder; the activity of the specific payload employed; the specific composition employed; the age, body weight, general health, sex and diet of the patient: the time of administration, route of administration; the duration of the treatment; drugs used in combination or coincidental with the specific modified host cell employed; and like factors well known in the medical arts.

In certain embodiments, modified host cell pharmaceutical compositions in accordance with the present disclosure may be administered at dosage levels sufficient to deliver from, e.g., about 1×10⁴ to 1×10⁵, 1×10⁵ to 1×10⁶, 1×10⁶ to 1×10⁷, or more modified cells to the subject, or any amount sufficient to obtain the desired therapeutic or prophylactic, effect. The desired dosage of the modified host cells of the present disclosure may be administered one time or multiple times. In some embodiments, delivery of the modified host cell to a subject provides a therapeutic effect for at least 1 month, 2 months, 3 months, 4 months, 5 months, 6 months, 7 months, 8 months, 9 months, 10 months, II months, 1 year, 13 months, 14 months, 15 months, 16 months. 17 months, 18 months, 19 months, 20 months, 21 months, 22 months, 23 months, 2 years, 3 years, 4 years, 5 years, 6 years, 7 years, 8 years, 9 years, 10 years or more than 10 years.

The modified host cells may be used in combination with one or more other therapeutic, prophylactic, research or diagnostic agents, or medical procedures, either sequentially or concurrently. In general, each agent will be administered at a dose and/or on a time schedule determined for that agent.

Use of a modified mammalian host cell according to the present disclosure for treatment of alpha-thalassemia, beta-thalassemia, sickle cell disease, hemophilia B, phenylketonuria, mucopolysaccharidosis type 1, Gaucher disease. Krabbe disease or other genetic conditions or disorders is also encompassed by the disclosure.

The present disclosure also contemplates kits comprising compositions or components of the present disclosure, e.g., sgRNA, Cas9, RNPs, i53, and/or homologous templates, as well as, optionally, reagents for, e.g., the introduction of the components into cells. The kits can also comprise one or more containers or vials, as well as instructions for using the compositions in order to modify cells and treat subjects according to the methods described herein.

### 7. RBC Production and Large-Scale Manufacturing

The methods and compositions disclosed herein can be used to produce red blood cells (RBCs) in vitro, in small, medium, or large scale as needed. In some embodiments, the method comprises: (i) editing one or more HSPCs to express at least one of the chimeric transmembrane receptor polypeptides as disclosed herein; and (ii) contacting the one or more HSPCs with a dimerization signal. In some embodiments, the dimerization signal is a small molecule dimerization signal. In some embodiments, the small molecule dimerization signal is AP20187 (BB dimerizer). In some embodiments, step (ii) further comprises contacting the one or more HSPCs with erythropoietin (EPO). In particular embodiments, the method is conducted in a bioreactor to produce RBCs at large scale.

In manufacturing large-scale RBCs at clinical requirements, pre-harvest RBC sample aliquots may be taken to establish cell counts, viability, cell characterization, e.g., FACs analysis, purity, and/or other general release criteria for the cells. In addition, post-harvest sample aliquots may be taken to establish cell counts and/or viability.

In some embodiments, the populations of RBCs are then transferred to one or more IV bags or other suitable vessels and cryopreserved in a controlled rate freezer until the cells are ready for use. In particular embodiments, cells are frozen in 50% plasmalyte and 50% Cryostor 10; 50/40/10 (XVIVO/HABS/DMSO); Crytostor 5 or Cryostor 10.

In some embodiments, bags (e.g., 10 to 250 mL capacity) containing RBCs are stored in blood bank conditions in a monitored −80 °C to −135 °C. Infusion bags can be stored in the freezer until needed.

The method disclosed herein can be used generate RBCs at a total cell number that is appropriate for the treatment of a subject in need thereof. In some embodiments, the treatment is a blood transfusion. In some embodiments, the scale of said method can result in a production of from about 1 million RBCs to about 200 million RBCs. In some embodiments, the scale of said method can result in a production of about 1 million, about 2 million, about 3 million, about 4 million, about 5 million, about 6 million, about 7 million, about 8 million, about 9 million, about 10 million, about 20 million, about 50 million, about 100 million, about 150 million, or about 200 million RBCs.

Illustrative embodiments of cell culture bags include, but are not limited to, MACS® GMP Cell Expansion Bags, MACS® GMP Cell Differentiation Bags, EXP-Pak™ Cell Expansion Bio-Containers, VueLife™ bags, KryoSure™ bags, KryoVue™ bags, Lifecell® bags, PermaLife™ bags, X-Fold™ bags, Si-Culture™ bags, Origen biomedical cryobags, and VectraCell™ bags. In particular embodiments, cell culture bags comprise one or more of the following characteristics: gas permeability (materials have suitable gas transfer rates for oxygen, carbon dioxide and nitrogen); negligible water loss rates (materials are practically impermeable to water); chemically and biologically inert (materials do not react with the vessel contents), and retention of flexibility and strength in various conditions (materials enable vessel to be microwaved, treated with UV irradiation, centrifuged, or used within a broad range of temperatures, e.g., from −100 °C to +100 °C).

Exemplary large-scale volumes of the cell culture vessel contemplated herein include, without limitation, volumes of about 10 mL, about 25 mL, about 50 mL, about 75 mL, about 100 mL, about 150 mL, about 250 mL, about 500 mL, about 750 mL, about 1000 mL, about 1250 mL, about 1500 mL, about 1750 mL, about 2000 mL, or more, including any intervening volume. For example, intervening volumes between 10 mL and 25 mL, include 11 mL, 12 mL, 13 mL, 14 mL, 15 mL, 16 mL, 17 mL, 18 mL, 19 mL, 20 mL, 21 mL, 22 mL, 23 mL, and 24 mL. In one embodiment, the volume of the cell culture vessel is from about 100 mL to about 500 L. In some embodiments, the method of manufacturing is prepared in a volume of at least 10L-500 L. In some embodiments, the method is prepared in a volume of at least 10 L, 20 L, 50L, 100 L, 250 L, or 500 L.

### 8. Examples

The present disclosure will be described in greater detail byway of specific examples. The following examples are offered for illustrative purposes only and are not intended to limit the disclosure in any manner. Those of skill in the art will readily recognize a variety of noncritical parameters which can be changed or modified to yield essentially the same results.

### Example 1. FKBP-EPOR Chimeras Place EPOR Signaling Under the Control of AP20187 (BB Dimerizer)

The EPOR signaling cascade is initiated by the presence of the EPO cytokine, which results in dimerization of two EPOR monomers. Prior work demonstrated the ability of engineered FKBP domains to mediate ligand-inducible dimerization of protein subunits (Martin, et al. Nature Communications, 2020). Therefore, we hypothesized that FKBP domains could be combined with EPOR to place the EPOR signaling cascade under exclusive regulation of an FDA-approved and orally bioavailable small molecule (AP20187, hereafter termed “BB dimerizer”).

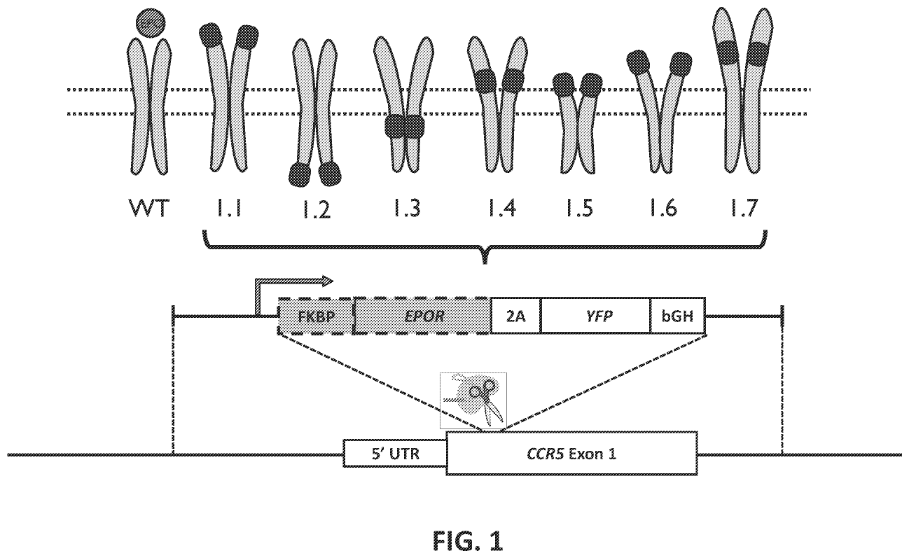

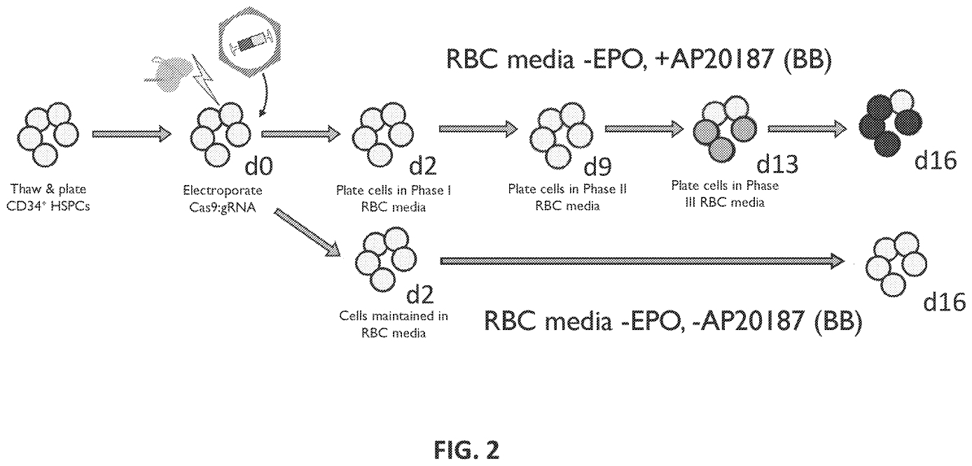

To determine if this is possible, we developed a series of integration constructs that added FKBP to the EPOR protein in a variety of conformations (FIG. 1 and Table 1). Each of these were then integrated into an AAV repair template that, via site-specific integration mediated by CRISPR, could be introduced to the CCR5 safe harbor site. Each of these initial constructs included a constitutive SFFV promoter driving the FKBP-EPOR chimeras followed by a 2A-YFP-bGH fluorescent reporter construct. We then edited WT human primary CD34+HSPCs using electroporation of Cas9 with gRNA targeting CCR5 followed by transduction with AAV6 to deliver the integration cassette DNA repair vectors, as indicated in FIG. 2.

Two days post-editing, we placed HSPCs in differentiation media, which is comprised of three phases of media over the course of 14 days (Dulmovits et al., 2016). Then HSPCs were cultured in RBC media without EPO and split into media with or without BB dimerizer. We hypothesized that RBCs should only be generated in media with BB dimerizer and the vast majority of these RBCs would be YFP+. To determine whether we achieved BB-dependent activation of each receptor, we compared the ability of cells from the same donor that were edited as a single treatment to differentiate into erythroid cells in the presence or absence of BB dimerizer. (FIG. 2). Although not shown in the diagram, in the day 16 sample with BB dimerizer we observe a lot more cells than in the sample without BB dimerizer, indicating a dramatic expansion of cells in the presence of BB dimerizer.

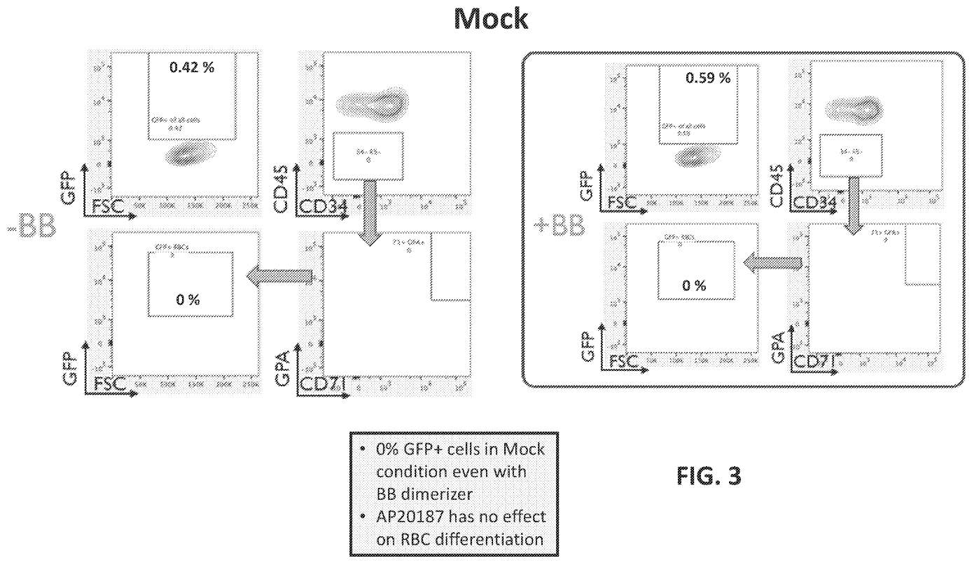

Importantly, we show that BB alone in the Mock condition (WT unedited cells) does not promote erythroid differentiation (FIG. 3), as indicated by the absence of CD45− cells at day 14. Therefore, BB dimerizer alone does not promote erythroid differentiation in WT HSPCs.

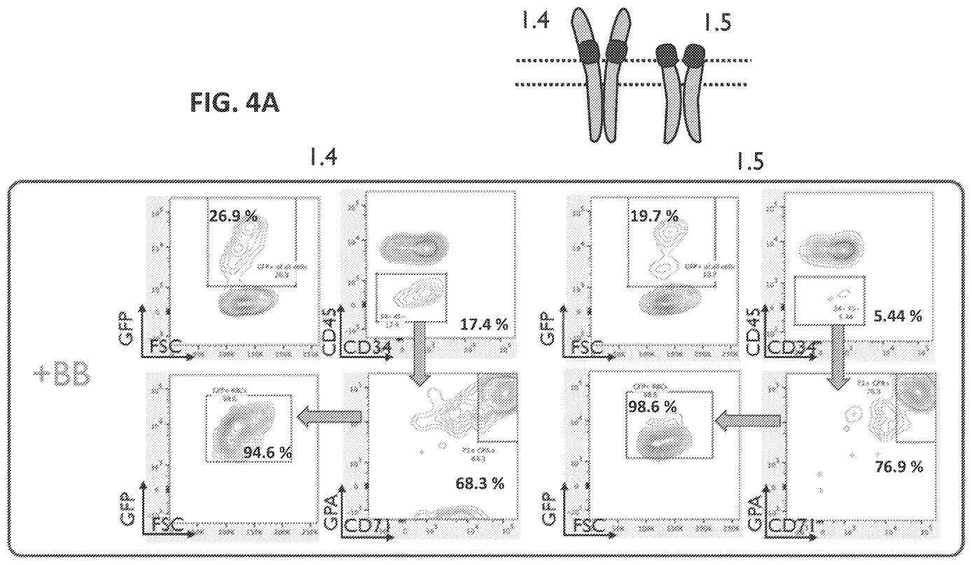

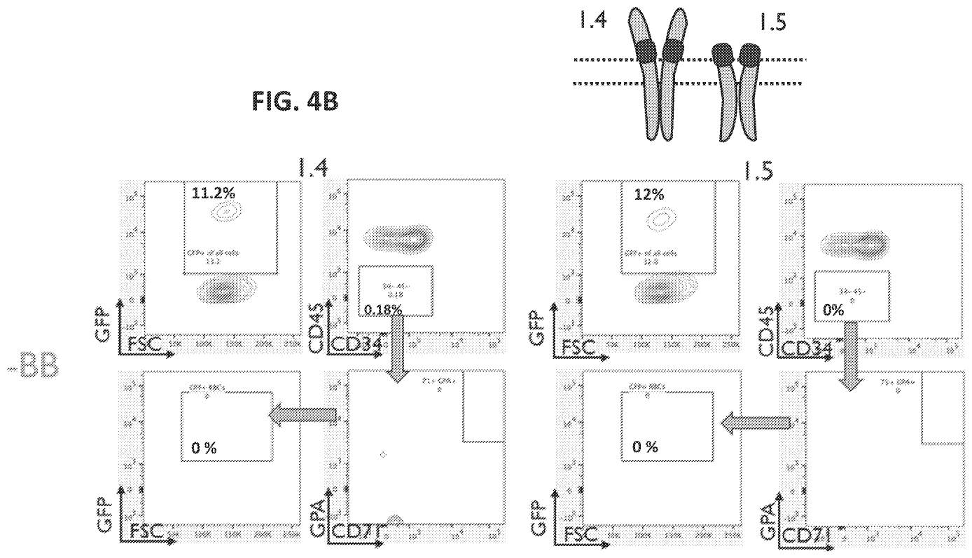

However, we do see BB dimerizer-driven RBC differentiation in the presence of BB dimerizer for constructs 1.4 and 1.5, which place the FKBP domain immediately adjacent to the transmembrane domain on the extracellular side of the receptor. About 11-12% of all cells in the samples in the absence of BB dimerizer are GFP+ at day 14, suggesting that baseline editing has occurred, yet none of these differentiate into RBCs (FIG. 4B). In contrast, about 19-27% of all cells in samples with BB dimerizer are GFP+ and nearly 100% of RBCs (CD34-CD45-CD71+ cells) are GFP+ (FIG. 4A). This indicates strong RBC-specific differentiation and expansion for cells edited with 1.4 and 1.5 in the presence of BB dimerizer. This also indicates that BB dimerizer can act as a substitute for EPO cytokine and drive RBC differentiation of cells containing chimeric receptors.

Enrichment of GFP+ cells in the bulk population, indicating preferential expansion of edited cells, was particularly noticeable in cells expressing either construct 1.4 or 1.5 (FIG. 5, left panel).

### Example 2. Optimizing Construct 1.5

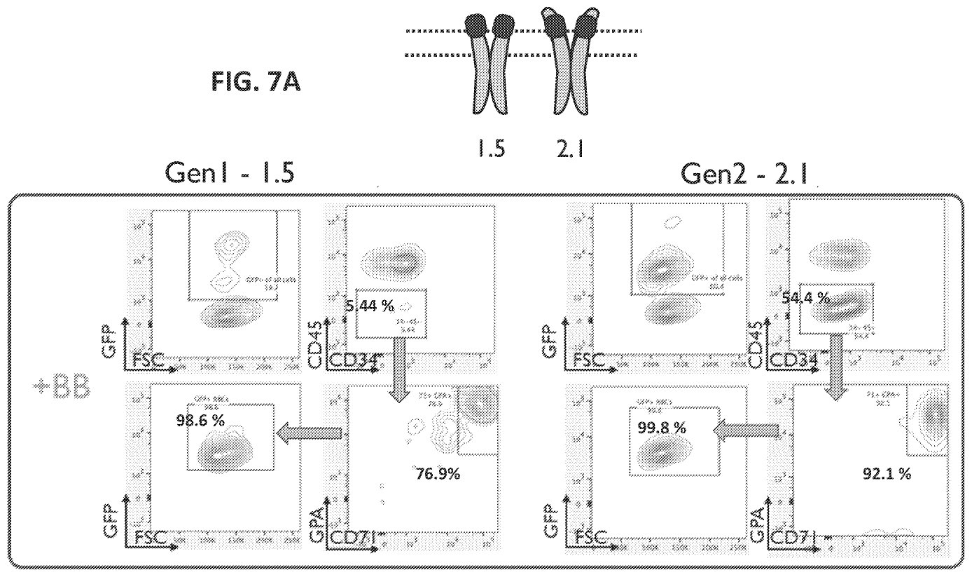

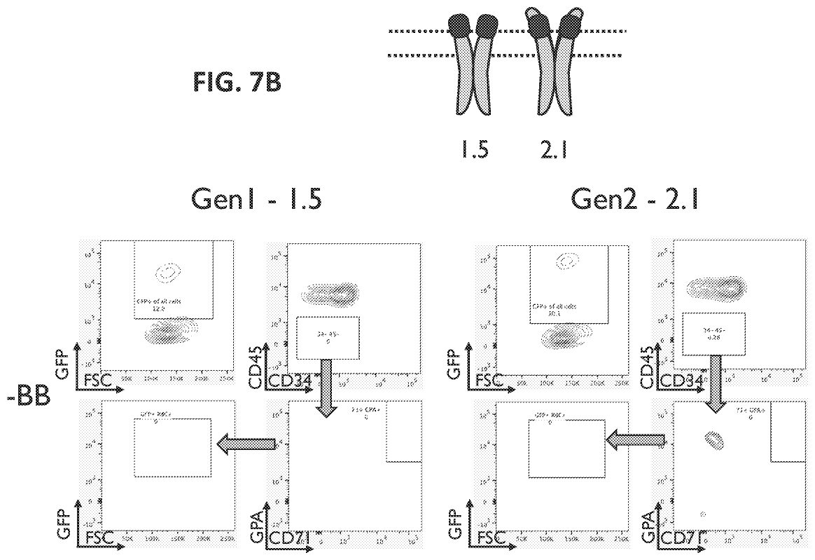

We next chose to optimize construct 1.5 because 1) it is a smaller cassette, which can be more easily included in an AAV repair template, and 2) it fully removes the EPOR extracellular domain, ensuring that the receptor is no longer responsive to EPO cytokine (see, e.g., FIG. 5, right panel). The improvements we tested included the addition of either native (abbreviated “SP”) (FIG. 6A) or IL6 signal peptides (FIG. 6B), which should ensure appropriate localization to the cell surface. We tested integration of the truncated EPOR receptor at the RBC-specific HBA1 safe harbor locus, which is expected to elicit strong, erythroid-specific expression (FIG. 6C). Indeed, following editing of WT HSPCs and erythroid differentiation in the absence of EPO and +/−BB, all three modifications improve the ability of 1.5 to facilitate BB-dependent erythroid differentiation. In each case, this is indicated by overall increases in RBC differentiation, a greater percentage of GFP+ cells, and a high percentage of RBCs among the GFP+ cells relative to cells with the original 1.5 construct. FIG. 7, for example, shows a representative comparison of original 1.5 to an IL6-containing version, 2.1. With addition of the signal peptide, we see even greater RBC differentiation and about 6 times the percentage of GFP+ cells in the +BB condition (FIG. 7A) relative to the −BB condition (FIG. 7B).

We also examined the degree to which induction of our optimized FKBP-EPOR constructs initiate differentiation in the absence of other cytokines known to promote an erythroid differentiated cell state. To do so, we kept HSPCs edited with IL6 or native SP peptide-containing FKBP-EPOR cassettes (MKC120 and MCK121) without EPO and with or without BB dimerizer in Porteus HSPC ex vivo culture media (Dever et al., Nature 2016), media known to preserve HSPC stem-ness. Cells were maintained at 100K cells/mL for 7 days post-editing in this media and then stained for the RBC antibody panel. As shown in FIGS. 8A and 8B, in spite of signals to maintain stem-ness and the absence of other RBC-promoting cytokines, BB-driven activation of our inducible EPOR cassettes promote differentiation into erythroid cells. This indicates that we have developed a strong, pro-RBC differentiation strategy that is activated by a small molecule, the BB dimerizer, rather than BB dimerizer simply acting as a proliferation switch that is guided toward RBC differentiation by the cytokines included in the RBC differentiation media.

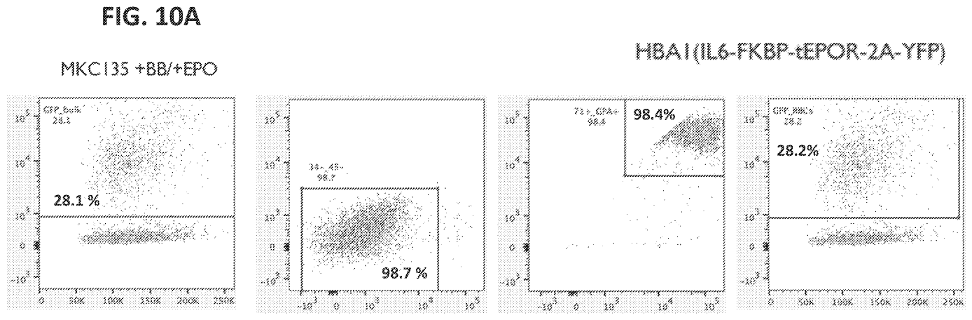

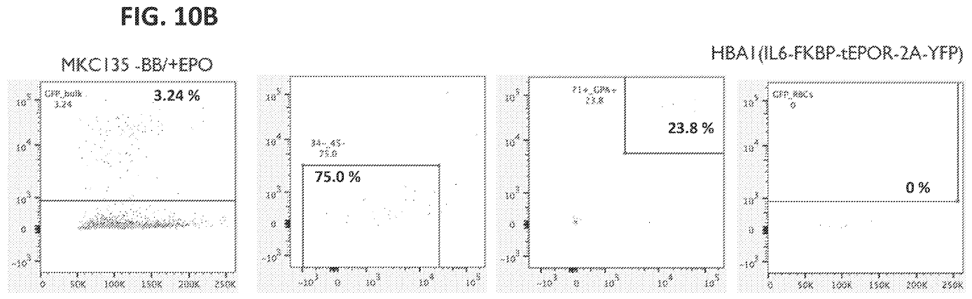

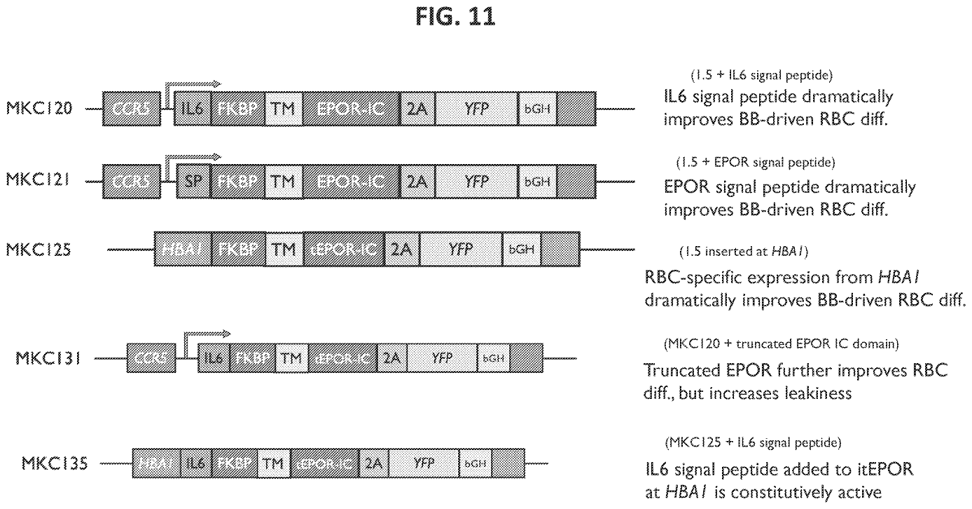

Due to the success of both the addition of a signal peptide as well as integration into the HBA1 locus (albeit with a cassette lacking a signal peptide) to improve on the original 1.5 construct, we then combined these optimizations into a single editing vector which integrates an IL-6-containing FKBP-EPOR at the HBA1 locus. This construct, termed MKC135, actually is so potent that it results in BB-independent RBC differentiation (FIGS. 9-12). We believe this is because expression of an iEPOR (IL6-FKBP-EPOR) from the HBA1 locus results in supraphysiological levels of this receptor translocating to the cell surface. As a consequence, these chimeric EPOR monomers may incidentally collide and activate some degree of EPOR signaling even in the absence of BB dimerizer. While a BB-independent, constitutively active chimeric EPOR has perhaps limited utility, we include the data here to demonstrate how quickly several design-build-test cycles can achieve extremely high-functioning signaling receptors. Furthermore, there are myriad ways to dial back expression and reduce hyper-function of this chimeric receptor in order to bring it back under control of our dimerizing small molecule.

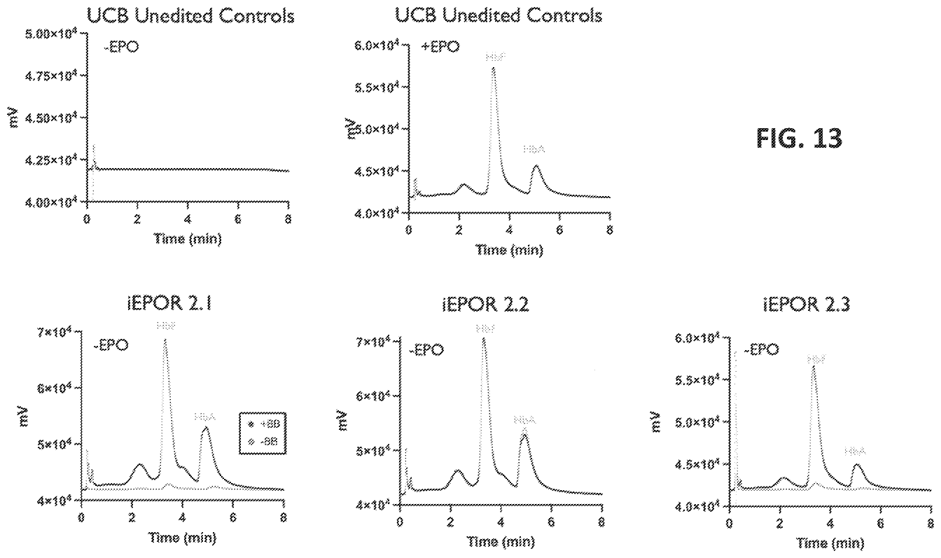

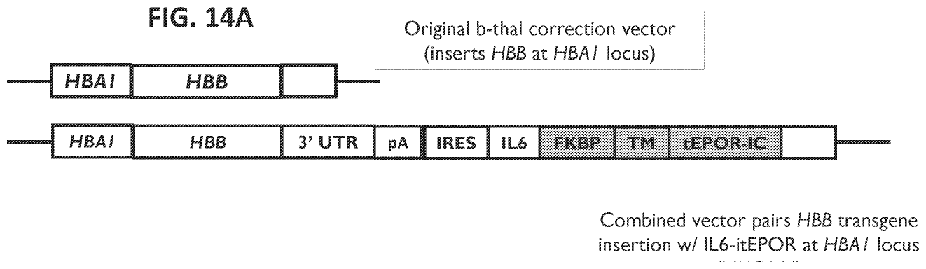

Following genome editing of healthy donor HSPCs, we further performed in vitro RBC differentiation followed by hemoglobin tetramer HPLC which enables quantification of primarily fetal hemoglobin (HbF) and adult hemoglobin (HbA) based on area under the curve. As shown in FIG. 13, unedited umbilical cord blood-derived (UCB) controls without EPO (−EPO, top left) produce no hemoglobin, while unedited cells cultured with EPO (+EPO, top right) produce significant HbF and HbA. Cells edited with iEPOR 2.1, iEPOR 2.2, or iEPOR 2.3 without EPO (−EPO, shown as bottom three panels, respectively) produce normal hemoglobin with addition of BB. Cells edited with iEPOR 2.2 even become “leaky” and produce high quantities of hemoglobin in absence of EPO and BB. These hemoglobin tetramer HPLC data shows that using BB alone can generate functional RBCs with hemoglobin production equivalent to unedited cells +EPO, indicating that dependence of EPO in induction of functional RBCs can be replaced with BB-induced erythropoiesis in these genomic edited cells.

One such way to both 1) dial back expression of the hyper-functioning construct in MKC135 and 2) to combine this inducible EPOR with a corrective clinical edit is to develop a bicistronic vector that integrates both the beta-thalassemia correction scheme (integrating a beta-globin transgene at HBA 1) as well as our iEPOR (IL6-FKBP-EPOR) cassette. By linking expression of these two genes by an internal ribosome entry site (IRES), and placing the chimeric EPOR downstream of the IRES, we expect to reduce expression of our RBC drive such that it resumes BB dimerizer dependence. This cassette is depicted in FIGS. 14A and 15A. Following editing of WT HSPCs with this cassette to create cell line MKC144 and differentiation into RBCs, we observe strong, BB-dependent RBC differentiation, as shown in FIG. 14B. Some degree of differentiation was observed in the −BB condition, indicating perhaps leaky dimerization caused by expression from the strong HBA1 promoter. Nonetheless, this cassette could potentially both correct the molecular pathology of beta-thalassemia and promote BB dimerizer-dependent RBC differentiation of edited cells.

We then quantified editing frequencies by ddPCR and show BB-dependent enrichment of edited cells in two separate WT HSPC donors over the 14-day course of RBC differentiation. (FIG. 15). % targeted cells for cells from each of two donors was determined by ddPCR in the presence and absence of BB dimerizer. As shown in FIG. 15B, middle and right panel, significant editing was observed in the presence of BB dimerizer relative to samples without BB dimerizer. Almost 100% of edited cells were able to differentiate into RBCs based on flow cytometry using antibodies to CD34, CD45, CD71, and GPA (FIG. 15B, left panel).

We next performed hemoglobin tetramer HPLC to further confirm that BB alone can generate functional RBCs with hemoglobin production equivalent to unedited cells +EPO. As shown in FIG. 15C, these genomic edited cells, when cultured with BB but without EPO, can produce a hemoglobin tetramer profile that resembles natural production from unedited cells cultured with EPO, indicating that dependence of EPO in induction of functional RBCs can be replaced with BB-induced erythropoiesis in these genomic edited cells.

Because it was possible that no iEPOR will better mimic native EPO signaling than integration at the endogenous EPOR locus, we next tested the effects of integrating an inducible chimeric receptor transgene at the EPOR locus. The general scheme is depicted in FIG. 16A. For these experiments, we integrated inducible truncated EPOR (itEPOR) at the endogenous EPOR locus by cutting the 3′ end of EPOR (gRNA sequence: AGCTCAGGGCACAGTGTCCA). This places itEPOR under the regulation of the endogenous EPOR promoter without disrupting the endogenous EPOR gene. The resulting cells displayed a dramatic BB dimerizer-dependent increase in the number of CD34-CD45-CD71+ RBCs (compare with and without BB dimerizer, FIGS. 16B and 16C, respectively). Other variations that are likely to work comprise cutting the 3′ UTR and integrating iEPOR or itEPOR there in order to keep endogenous EPOR intact.

Another variation we tested was an inducible chimeric receptor containing truncated EPOR (tEPOR) integrated at the CCR5 locus but with expression driver by the PGK promoter. Although this polypeptide also caused BB dimerizer-dependent increase in CD34-CD45-CD71+ RBCs, expression from the PGK promoted construct was difficult to detect overall (FIG. 16A last panel, trying to detect GFP+ cells in sample with BB dimerizer).

To ensure that activation of iEPOR or itEPOR resulted in RBC's that were functionally normal in terms of their hemoglobin production, hemoglobin was extracted from edited and differentiated RBCs and analyzed by HPLC. For reference, FIG. 18A shows unedited umbilical cord blood-derived HSPCs from a donor (“CB Mock”) that were differentiated in RBC media with Epo. Fetal and adult hemoglobin are labeled as HbF and HbA, respectively.

FIG. 18B shows the corresponding HPLC profiles of hemoglobin extracted from cells incubated with (black) or without (gray) BB dimerizer after editing to introduce SSF-IL6-FKBP-EPOR integrated at the CCR5 locus (MCK120), SSFV-SP-FKBP-EPOR integrated at the CCR5 locus (MKC121), SSFV-IL6-FKBP-tEPOR integrated at the CCR3 locus (MKC131), IL6-FKBP-tEPOR integrated at the HBA1 locus (MKC135), HBB-IRES-IL6-FKBP-tEPOR integrated at the HBA 1 locus (MKC144). A mock sample that lacks a construct encoding an inducible chimeric receptor is shown for comparison (Mock, bottom right). For all samples, we see HbF and HbA production that is equivalent in total amount and ratio of HbF:HbA to the CB Mock+EPO sample shown in FIG. 18A.

For cells edited with MKC120 and MKC135, the hemoglobin profile in the −BB samples closely resembled the hemoglobin profile in Mock samples −EPO and —BB at the bottom right of FIG. 18B. This shows that we have effectively replaced native EPO signaling with BB-inducible EPOR to create functionally normal RBCs. For cells edited with MKC120 and MKC135, we see hemoglobin production in the −BB samples, indicating some degree of leakiness (i.e., activation in the absence of BB dimerizer) of the iEPOR in these conditions.

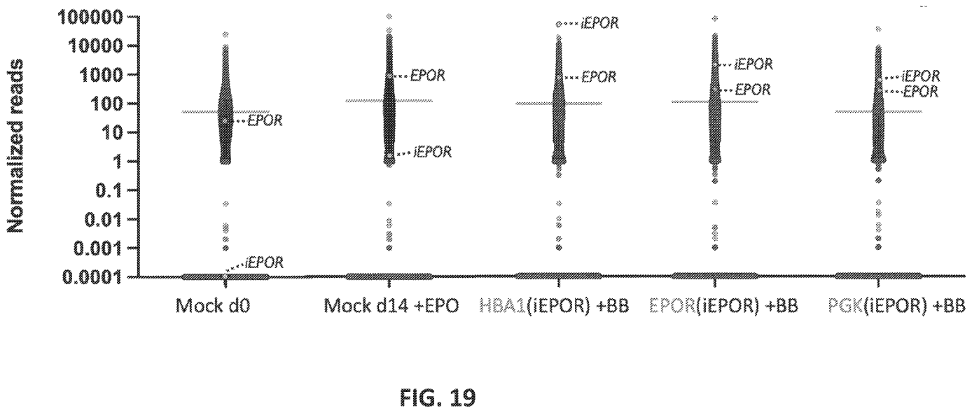

Following genome editing of healthy donor HSPCs and in vitro RBC differentiation, bulk RNA-Seq was performed to compare transcriptional response to native EPOR+EPO vs. iEPOR+BB. About 18,000 genes are RNA sequenced with assigned expression values shown as normalized read counts in FIG. 19. Native EPOR and iEPOR expression levels are annotated for Mock “unedited” cells at d0 and d14 of RBC diff (Mock d0 and Mock d14+ EPO) followed by cells edited with iEPOR version 2.3 expressed by strong-RBC specific promoter HBA1 (HBA1(iEPOR) +BB), endogenous EPOR promoter (EPOR(iEPOR) +BB), and constitutive-expressing hPGK promoter (PGK(iEPOR) +BB), as shown in FIG. 19. We find that native EPOR expression is lowly expressed at d0 but then elevated at consistent levels at d14 in all the cell samples, iEPOR expression shows the highest when driven by HBA1 promoter, while approach native expression levels when driven by native EPOR or PGK promoter.

Principal Component Analysis (PCA) was performed after bulk RNA-Seq, and we find that cells with iEPOR driven by native EPOR promoter cultured with BB but without EPO (−EPO/+BB) most closely resembles the expression profile of Mock d14 cells cultured +EPO. Based on PC1+2, cells with PKG promoter-driven iEPOR cultured with BB (+BB) has expression profile next similar to Mock d14 cells cultured with EPO (+EPO). Cells with HBA1 promoter-driven iEPOR, cultured with BB (+BB), are the most different from all other cell samples, likely due to the abnormally high levels of iEPOR expression. [01%]Following bulk RNA-Seq, we calculated which genes showed the greatest rank change when compared to Mock d0. We then took the top 1% of these genes with greatest rank change and used them as input into gene ontology (GO) enrichment analysis software. Tables 2-5 show the GO processes which achieve significant enrichment among each gene set. We find RBC-associated pathways to be highly enriched across all samples (Mock+EPO and all iEPOR+BB conditions) compared to Mock d0, indicating that we are able to use BB to replace dependence on EPO to produce cells that resemble natural RBCs at the transcriptional level. This data shows that native EPOR+EPO and iEPOR+BB reliably upregulate the same RBC-associated GO pathways, indicating that iEPOR+BB is making functional RBCs.

### Example 3. Analogous Chimeric Receptor Systems

Due to the reported modularity of other membrane-bound signaling receptors (such as EGFR, c-kit, and c-fins), we hypothesized that we could design analogous chimeric receptors to promote expansion of other clinically relevant cell types. These include the c-KIT protein (SCF) and thrombopoietin receptor (TPOR), activation of which has been reported to promote expansion of engraftable HSPCs (not shown). We also designed an equivalent vector for inducible activation of epidermal growth factor receptor (EGFR), which is known to promote differentiation to epithelial cells (FIG. 20). As before, these initial vectors were designed to integrate at safe harbor locus CCR5 and be expressed by the SFFV promoter.

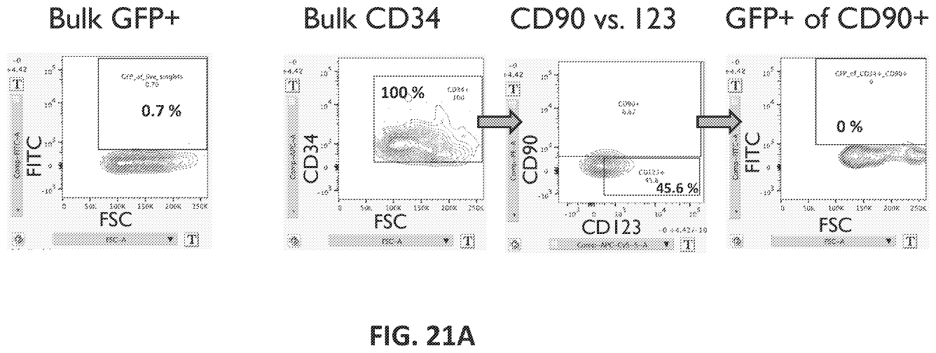

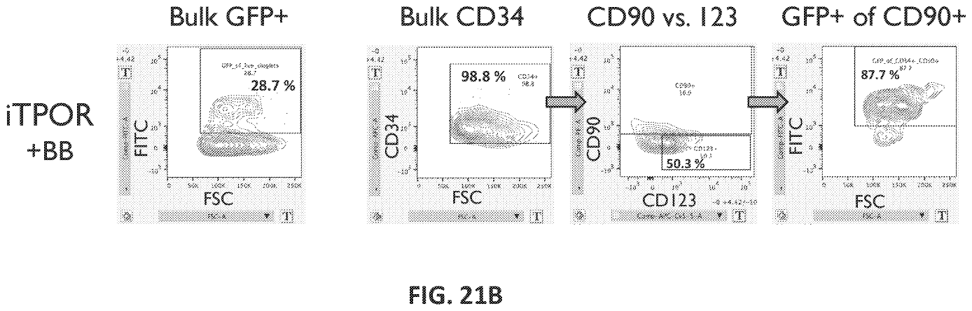

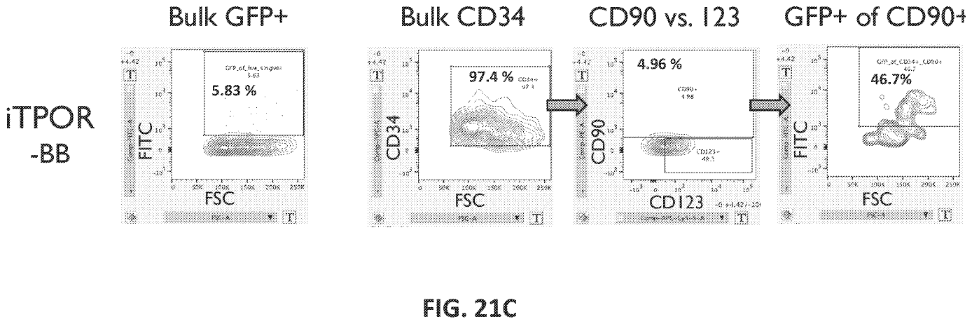

To test the effectiveness of our candidate HSPC drives, we edited WT HSPCs with both IL6-FKBP-SCF (iSCF) and IL6-FKBP-TPOR (iTPOR) and then placed cells into HSPC media with or without BB dimerizer for 7 days. Cells were then stained for the HSC-specific markers CD34, CD90, and CD123. No dramatic difference was observed for the HSC markers in the −BB and +BB samples (FIGS. 21B and 21C). This may be because detection of HSPCs is much more difficult than detection of RBCs. In part, this is because RBC staining is clearly visible over background staining, whereas HSC markers do not show great differences in brightness over background. Also, true long-term HSCs are fairly rare, so even there were noticeable differences from background staining, they may nonetheless be difficult to detect in a population of cells. However, we observe an approximately five-fold enrichment of % GFP+cells in the +BB (FIG. 21B) relative to −BB (FIG. 21C) samples. This indicates a functional effect likely driven by the effective dimerization of our chimeric TPOR cassette. Also, there is a large % of edited cells in the presence of BB dimerizer (FIG. 21B). By comparison, mock samples had a very low % of GFP+ cells (FIG. 21A). When quantifying fold change in GFP with addition of BB dimerizer, we see reproducible enrichment in bulk cells edited with the iTPOR cassette (FIGS. 22A and 22B), and modest enrichment in the condition edited with iSCF (FIG. 22C). Thus, addition of BB increased the proportion of edited cells in the population. These results were further confirmed at the genomic level by using ddPCR to quantify editing frequency in conditions+/−BB dimerizer.

We believe this concept could be applied to bias cells to produce a wide variety of stem and differentiated cell types for clinical purposes. For instance, macrophage differentiation is known to be driven by dimerization of a single membrane-bound signaling receptor, CSF1R. We believe the designs we have optimized here could be readily applied to create an inducible chimeric CSF1R expression cassette to bias edited HSPCs to preferentially differentiation into macrophages following transplantation.

### Example 4. Engineering Complex Signaling Pathways that are Tunable with Small Molecules

While our work only examined the potential of homodimerization, analogous FKBP-based heterodimerization domains also exist which are responsive to a separate small molecule. We believe these could be used to activate signaling pathways currently dependent on dimerization of two separate monomers. In fact, both engineered homo- and heterodimer cassettes could be introduced into a single cell to activate separate pathways with two separate, non-cross-reactive, small molecules. This paves the way for us to engineer increasingly complex signaling logic into cells in the future.

### Example 5. Treatment of Hemoglobinopathies

We believe these findings challenge the long-standing and prevalent dogma that HSPC chimerism in the BM must yield equivalent RBC chimerism in the bloodstream. We now have the potential to address one of the greatest barriers to effective treatment of the hemoglobinopathies. We also believe that our efforts to engineer tunable, inducible elements into living cells will lend an unprecedented level of control to cells post-transplant. These approaches could be immediately paired with current viral-based gene therapies as well as CRISPR-based genome editing strategies to reduce the threshold for curative levels of corrected HSPC chimerism in the BM. In addition, our inducible erythroid bias strategies could also be integrated into the allo-HSCT workflow to yield tunable RBC production from donor-derived HSPCs. For example, a patient's own HSPC or an HSPC from an allogeneic donor could be edited to correct an allele that causes a hemoglobinopathy such as beta-thalassemia or sickle cell anemia as well as to express a chimeric EPO receptor that is inducible by an orally available or locally administered small molecule. The edited HSPC could then be engrafted into the patient's bone marrow and, upon exposure to the small molecule, be allowed to preferentially expand and differentiate into a population of edited erythroid cells, thereby treating the hemoglobinopathy. This would, in effect, reduce or eliminate the need for high morbidity mycloablation regimens and improve the safety and accessibility to currently curative treatments for the large number of patients suffering from the hemoglobinopathies worldwide.

### Example 6. RBC Bioreactor Production

Given our ability to produce functional RBCs as validated by RNA-Seq and HPLC using BB in place of EPO, we hypothesized that this technology could be used to produce RBCs in a bioreactor while avoiding one of the most costly reagents currently required for production—purified recombinant human EPO cytokine.

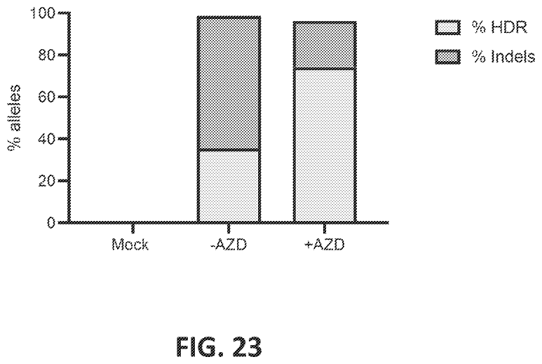

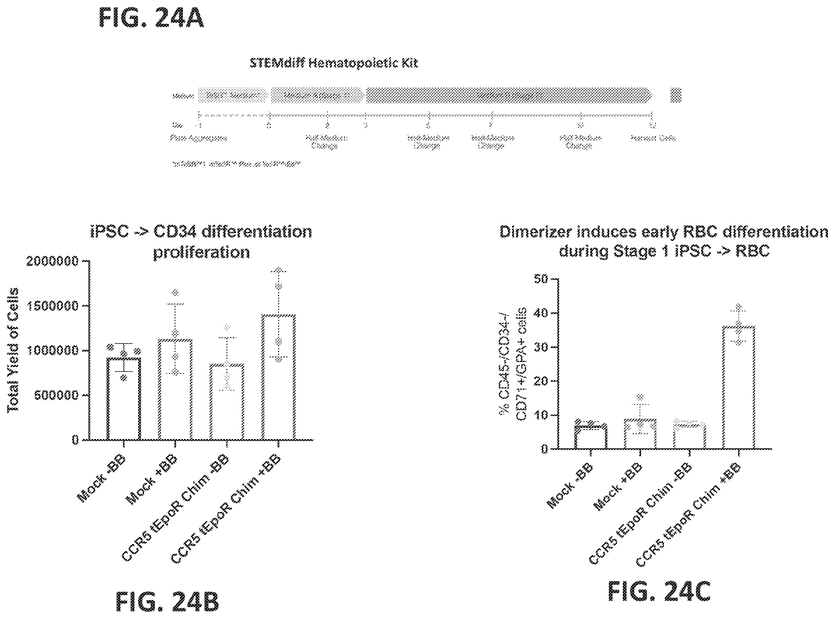

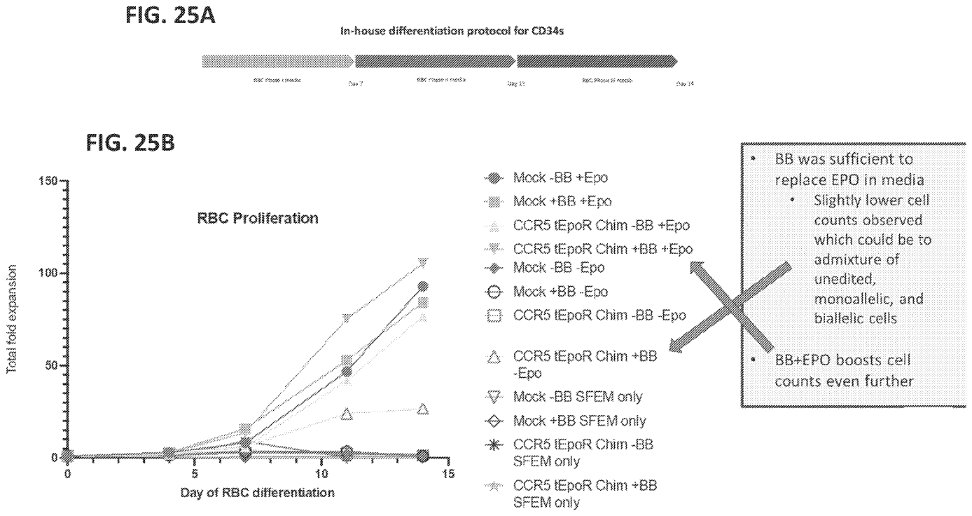

We first edited iPSCs with the PGK promoter-driven iEPORv2.3 and achieved highly efficient editing as depicted in FIG. 23. Next, we performed in vitro iPSC>RBC differentiation using STEMdiff Hematopoietic kit with the workflow as shown in FIG. 24A or using in-house differentiation protocol for CD34s with the workflow as shown in FIG. 25A. In the first phase of differentiation (iPSC>HPC) using STEMdiff Hematopoictic kit, we observed no major differences between unedited cells cultured with BB and iEPOR-edited cells cultured without BB (FIG. 24B). However, we observed a dramatic increase in cells acquiring RBC markers when iEPOR-edited cells are cultured with BB (FIG. 24C), indicating that some frequency of early differentiation is occurring that is driven by BB-induced iEPOR signaling in this condition. We also observed an increase in CD71+/GPA+ cells with BB which may be due to early differentiation of the cells (FIG. 24C).

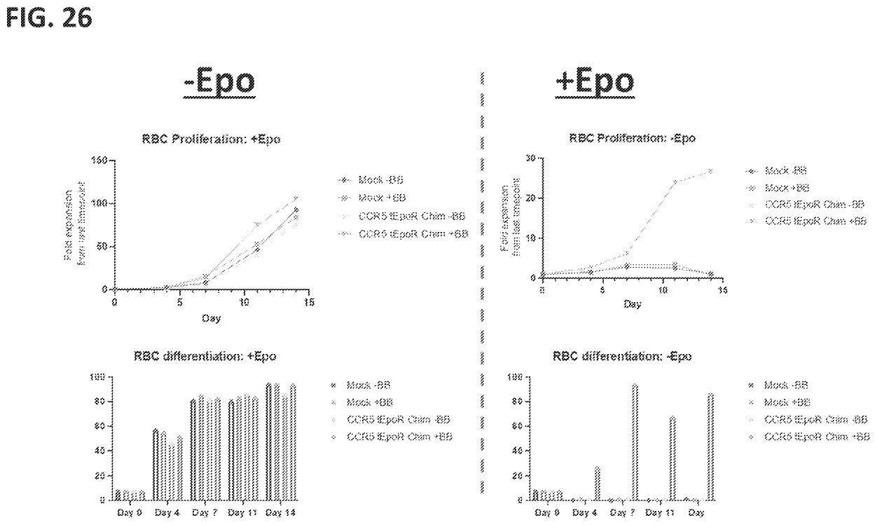

In vitro iPSC>RBC differentiation using in-house differentiation protocol for CD34s (FIG. 25A) further confirmed that BB is sufficient to replace EPO in media, and BB+EPO boosts cell counts even further. Over the course of HPC>RBC differentiation using in-house differentiation protocol for CD34s, we tracked total cell counts and observed dramatic increases in cell counts (as expected with effective erythropoiesis) in all cell samples cultured +EPO. We also observed the highest cell production in iEPOR-edited cells cultured +EPO/+BB, indicating that BB stimulation of iEPOR leads to an elevated boost even on top of natural EPO stimulation (FIG. 25B). We then discovered that iEPOR-edited cells cultured −EPO/+BB lead to an intermediate expansion of cells, indicating that BB can be used to replace EPO in the media and lead to substantial RBC production. In contrast, unedited and edited cells cultured −EPO and −BB lead to virtually no cell expansion, as expected for these negative controls (FIG. 25B). Top panels of FIG. 26 show the data in FIG. 25B split up between −EPO and +EPO conditions. We then stained cells throughout HPC>RBC differentiation for RBC markers and observed that in % of cells with RBC markers reaches almost 100% by d14 of RBC diff (FIG. 26). In absence of EPO, only iEPOR-edited cells +BB are able to effectively differentiate and acquire RBC markers (FIG. 26).

### Materials and Methods

### AAV6 Vector Design, Production, and Purification

All AAV6 vectors were cloned into the pAAV-MCS plasmid (Agilent Technologies, Santa Clara, CA, USA), which contains inverted terminal repeats (ITRs) derived from AAV2. Gibson Assembly Mastermix (New England Biolabs, Ipswich, MA, USA) was used for the creation of each vector as per manufacturer's instructions. Few modifications were made to the production of AAV6 vectors as described¹. 293T cells (Life Technologies, Carlsbad, CA, USA) were seeded in five 15 cm² dishes with 17×10⁶ cells per plate. 24 h later, each dish was transfected with a standard polyethylenimine (PEI) transfection of 6 μg ITR-containing plasmid and 22 μg pDGM6, which contains the AAV6 cap genes, AAV2 rep genes, and Ad5 helper genes. After a 48-72 h incubation, cells were purified using AAVPro Purification Kits (All Serotypes)(Takara Bio USA, Mountain View, CA, USA) as per manufacturer's instructions. AAV6 vectors were titered using ddPCR to measure number of vector genomes per L as previously described².

### Culturing of CD34⁺ HSVCs

Human CD34” HSPCs were cultured as previously described³⁻⁸. Healthy donor CD34⁺ HSPCs were sourced from fresh cord blood, frozen cord blood, and Plerixafor- and/or G-CSF-mobilized peripheral blood (AllCells, Alameda, CA, USA and STEMCELL Technologies, Vancouver, Canada). CD34⁺ HSPCs were cultured at 1-10 cells/mL in StemSpan SFEM II (STEMCELL Technologies, Vancouver, Canada) base medium supplemented with stem cell factor (SCF)(100 ng/mL), thrombopoietin (TPO)(100 ng/mL), FLT3-ligand (100 ng/mL), IL-6 (100 ng/mL), UM171 (35 nM), streptomycin (20 mg/mL), and penicillin (20U/mL). The cell incubator conditions were 37 °C, 5% CO₂, and 5% 02.

### Genome Editing of CD34⁺ HSPCs

Chemically modified Cas9 sgRNAs were purchased from Synthego (Menlo Park, CA, USA) and TriLink BioTechnologies (San Diego, CA, USA) and were purified by high-performance liquid chromatography (HPLC). The sgRNA modifications added were the 2′-O-methyl-3′-phosphorothioate at the three terminal nucleotides of the 5′ and 3′ ends described previously”. The target sequences for human sgRNAs were as follows: HBA1sg: 5′-GGCAAGAAGCATGGCCACCG-3′ (SEQ ID NO: 23): CCR5 sg: 5′-GCAGCATAGTGAGCCCAGAA-3′ (SEQ ID N-O: 24); HBB sg7: 5′-GGGTGGGAAAATAGACCAAT-3′ (SEQ ID NO: 25); HBB sg11: 5-TATGGTTAAGTTCATGTCAT-3′ (SEQ ID NO: 26); HBB sg13: 5-TAGGAAGGGGATAAGTAACA-3′ (SEQ ID NO: 27); and EPORsg: 5-AGCTCAGGGCACAGTGTCCA-3′ (SEQ ID NO: 28). All hi-fidelity variant¹⁰ Cas9 protein (SpyFi) was purchased from Aldevron, LLC (Fargo, ND, USA). The RNPs were complexed at a Cas9:sgRNA molar ratio of 1:2.5 at 25 °C for 10 min prior to electroporation. CD34⁺ cells were resuspended in P3 buffer (Lonza, Basel, Switzerland) with complexed RNPs and electroporated using the Lonza 4D Nucleofector (program DZ-100). Cells were plated at 1×10⁵ cells/mL following electroporation in the cytokine-supplemented media described previously. Immediately following electroporation, AAV6 was supplied to the cells at 5×10³ vector genomes/cell based on titers determined by ddPCR.

### Gene Targeting Analysis by Flow Cytometry

For targeting analysis by flow cytometry, CD34′ HSPCs were harvested at day 5 and erythrocytes at day 16 post-targeting. Cells were analyzed for viability using Ghost Dye Red 780 (Tonbo Biosciences, San Diego, CA, USA) and reporter expression was assessed using the FACS Aria II system (BD Biosciences, San Jose, CA, USA). The data was subsequently analyzed using FlowJo (FlowJo LLC. Ashland, OR, USA)

In Vitro Differentiation of CD34⁺ HSPCs into Erythrocytes

Following targeting, HSPCs derived from healthy donors or polycythemia vera patients were cultured for 14-16 days at 37 °C and 5% CO₂ in SFEM II medium (STEMCELL Technologies, Vancouver, Canada). SFEMII base medium was supplemented with 100 U/mL penicillin-streptomycin, 10 ng/mL SCF, 1 ng/mL IL-3 (PeproTech, Rocky Hill, NJ, USA), 3U/mL erythropoietin (eBiosciences, San Diego, CA, USA). 200 μg/mL transferrin (Sigma-Aldrich, St. Louis, MO, USA), 3% antibody serum (heat-inactivated from Atlanta Biologicals, Flowery Branch, GA, USA), 2% human plasma (derived from umbilical cord blood), 10 μg/mL insulin (Sigma-Aldrich, St. Louis, MO, USA), and 3U/mL heparin (Sigma-Aldrich, St. Louis, MO, USA). In the first phase, days 0-7 (day 0 being 2 days post-targeting) of differentiation, cells were cultured at 1×10⁵ cells/mL. In the second phase, days 7-10, cells were maintained at 1×10⁵ cells/mL, and IL-3 was removed from the culture. In the third phase, days 11-16, cells were cultured at 1×10⁶ cells/mL, and transferrin was increased to 1 mg/mL within the culture medium.

### Immunophenotyping of Differentiated Erythrocytes

HSPCs subjected to the above erythrocyte differentiation were analyzed at day 14 for erythrocyte lineage-specific markers using a FACS Aria II (BD Biosciences, San Jose, CA, USA). Edited and non-edited cells were analyzed by flow cytometry using the following antibodies: hCD45 V450 (H130; BD Biosciences, San Jose, CA. USA), CD34 APC (561; BioLegend, San Diego, CA, USA), CD71 PE-Cy7 (OKT9; Affymetrix, Santa Clara, CA, USA), and CD235a PE (GPA)(GA-R2; BD Biosciences, San Jose, CA, USA).

### Indel Frequency Analysis by TIDE

2-4 days post-targeting, HSPCs were harvested and QuickExtract DNA extraction solution (Epicentre, Madison, WI, USA) was used to collect gDNA. Primers were then used to amplify the region surrounding the predicted cut site and/or deletion. PCR reactions were then run on a 1% agarose gel and appropriate bands were cut and gel-extracted using a GeneJET Gel Extraction Kit (Thermo Fisher Scientific, Waltham, MA, USA) according to manufacturer's instructions. Gel-extracted amplicons were then Sanger sequenced and resulting chromatograms were used as input for indel frequency analysis by TIDE as previously described “.

Allelic Targeting Analysis by ddPCR

2-4 days post-targeting, HSPCs were harvested and QuickExtract DNA extraction solution (Epicentre, Madison. WI, USA) was used to collect gDNA, gDNA was then digested using BamHI-HF as per manufacturer's instructions (New England Biolabs, Ipswich, MA, USA). The percentage of targeted alleles within a cell population was measured by ddPCR using the following reaction mixture: 1-4 μL of digested gDNA input, 10 μL ddPCR SuperMix for Probes (No dUTP)(Bio-Rad, Hercules, CA, USA), primer/probes (1:3.6 ratio; Integrated DNA Technologies, Coralville, Iowa, USA), volume up to 20 μL with H₂O. ddPCR droplet were then generated following the manufacturer's instructions (Bio-Rad, Hercules, CA, USA): 20 μL of ddPCR reaction, 70 μL droplet generation oil, and 40 μL of droplet sample. Thermocycler (Bio-Rad, Hercules, CA, USA) settings were as follows: 1.98 °C (10 min), 2. 94 °C (30s), 3. 57.3 °C (30s), 4.72 °C (1.75 min)(return to step 2×40-50 cycles), 5.98 °C (10 min). Analysis of droplet samples was done using the QX200 Droplet Digital PCR System (Bio-Rad, Hercules, CA, USA). To determine percentage of alleles targeted, the number of Poisson-corrected integrant copies/mL were divided by the number of Poisson-corrected reference DNA copies/mL.

mRNA Analysis

Following differentiation of HSPCs into erythrocytes, cells were harvested and RNA was extracted using RNeasy Plus Mini Kit (Qiagen, Hilden, Germany). Subsequently, cDNA was made from approximately 100 ng RNA using the iScript Reverse Transcription Supermix for RT-qPCR (Bio-Rad, Hercules, CA, USA). Expression levels of EPOR mRNA were quantified by ddPCR using specific primers and 6-FAM/ZEN/IBFQ-labelled hydrolysis probes purchased as custom-designed PrimeTime qPCR Assays from Integrated DNA Technologies (Coralvilla, IA, USA). To normalize for RNA input, levels of the RBC-specific reference gene GPA was determined in each sample using the following primers and HEX/ZEN/IBFQ-labelled hydrolysis probes purchased as custom-designed PrimeTime qPCR Assays from Integrated DNA Technologies (Coralvilla, IA, USA): forward: 5′-ATATGCAGCCACTCCTAGAGCTC-3′, reverse: 5-CTGGTTCAGAGAAATGATGGGCA-3′, probe: 5′-AGGAAACCGGAGAAAGGGTA-3′. ddPCR reactions were created using the respective primers and probes and droplets were generated as described above. Thermocycler (Bio-Rad, Hercules, CA, USA) settings were as follows: 1.98 °C (10 min), 2.94 °C (30s), 3. 59.4 °C (30s), 4.72 °C (30s)(return to step 2-40-50 cycles), 5.98 °C (10 min). Analysis of droplet samples was done using the QX200 Droplet Digital PCR System (Bio-Rad, Hercules, CA, USA). To determine relative expression levels, the number of Poisson-corrected HBA or HBB transgene copies/mL were divided by the number of GPA copies/mL.

### Steady-State Hemoglobin Tetramer Analysis

HSPCs subjected to erythrocyte differentiation were lysed using water equivalent to three volumes of pelleted cells. The mixture was incubated at room temperature for 15 min, followed by 30s sonication. For separation of lysate from the erythrocyte ghosts, centrifugation was performed at 13,000 RPM for 5 min. HPLC analysis of hemoglobins in their native form were analyzed on a weak cation-exchange PolyCAT A column (100×4.6-mm, 3 μm, 1,000 Å) (PolyLC Inc., Columbia. MD, USA) using a Shimadzu UFLC system at room temperature. Mobile phase A (MPA) consists of 20 mM Bis-tris+2 mM KCN, pH 6.96. Mobile phase B (MPB) consists of 20 mM Bis-tris+2 mM KCN+200 mM NaCl, pH 6.55. Clear hemolysate was diluted four times in MPA, and then 20 μL was injected onto the column. A flow rate of 1.5 mL/min and the following gradients were used in time (min)/% B organic solvent: (0/10%: 8/40%: 17/90%; 20/10%; 30/stop).

### REFERENCES

1. Khan, I F., Hirata. R. K. & Russell, D. W. AAV-mediated gene targeting methods for human cells. Nat Proc 6, 482-501 (2011).
2. Aurnhammer, C. et al. Universal real-time PCR for the detection and quantification of adeno-associated virus serotype 2-derived inverted terminal repeat sequences. Hum Gene Ther Methods 23, 18-28 (2012).
3. Dever, D. P. et al. CRISPR/Cas9 beta-globin gene targeting in human haematopoietic stem cells. Nature 539, 384-389 (2016).
4. Charlesworth, C. T. et al. Priming Human Repopulating Hematopoietic Stem and Progenitor Cells for Cas9/sgRNA Gene Targeting. Mol Ther Nucleic Acids 12, 89-104 (2018).
5. Bak, R. O., Dever, D. P. & Porteus, M. H. CRISPR/Cas9 genome editing in human hematopoietic stem cells. Nat Protoc 13, 358-376 (2018).
6. Bak, R. O. et al. Multiplexed genetic engineering of human hematopoietic stem and progenitor cells using CRISPR/Cas9 and AAV6. Elife 6 (2017).
7. Cromer, M. K. et al. Global Transcriptional Response to CRISPR/Cas9-AAV6-Based Genome Editing in CD34(+) Hematopoictic Stem and Progenitor Cells. Mol Ther 26, 2431-2442 (2018).
8. Bak, R. O. & Porteus, M. H. CRISPR-Mediated Integration of Large Gene Cassettes Using AAV Donor Vectors. Cell Rep 20, 750-756 (2017).
9. Hendel, A. et al. Chemically modified guide RNAs enhance CRISPR-Cas genome editing in human primary cells. Nat Biotechnol 33, 985-989 (2015).
10. Vakulskas, C. A. et al. A high-fidelity Cas9 mutant delivered as a ribonucleoprotein complex enables efficient gene editing in human hematopoietic stem and progenitor cells. Nat Med 24, 1216-1224 (2018).
11. Dulmovits, B. M. et al. Pomalidomide reverses gamma-globin silencing through the transcriptional reprogramming of adult hematopoietic progenitors. Blood 127, 1481-1492 (2016).
12. Hu, J. et al. Isolation and functional characterization of human erythroblasts at distinct stages: implications for understanding of normal and disordered erythropoiesis in vivo. Blood 121, 3246-3253 (2013).
13. Brinkman, E. K., et al. Easy quantitative assessment of genome editing by sequence trace decomposition. Nucleic Acids Res 42, e168 (2014).

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

TABLE 1

| Cell | | | Integration | |
|---|---|---|---|---|
| Line | Other names | Receptor | Site | Promoter |
| WT | Unedited, CD34+ HSPC | EC domain-TM-IC domain | n/a | EPOR |
| MKC111 | 1.1 | FKBP-EC domain-TM-IC domain-2A-YFP-bGH | CCR5 | SFFV |
| MKC112 | 1.2 | EC domain-TM-IC domain-FKBP-2A-YFP-bGH | CCR5 | SFFV |
| MKC113 | 1.3 | EC domain-TM-FKBP-IC domain-2A-YFP-bGH | CCR5 | SFFV |
| MKC114 | 1.4 | EC domain-FKBP-TM-IC domain-2A-YFP-bGH | CCR5 | SFFV |
| MKC115 | 1.5 | FKBP-TM-IC domain-2A-YFP-bGH | CCR5 | SFFV |
| MKC116 | 1.6 | EC-FKBP-EC domain-TM-IC domain-2A-YFP-bGH | CCR5 | SFFV |
| MKC117 | 1.7 | FKBP-EC-TM-IC domain-2A-YFP-bGH | CCR5 | SFFV |
| MKC118 | | FKBP-TM-tEPOR-IC-2A-YFP-bGH | CCR5 | PGK |
| MKC119 | | FKBP-LV-TM-EPOR-IC-2A-YFP-bGH | CCR5 | SFFV |
| MKC120 | 2.1 | IL6-FKBP-TM-EPOR-IC-2A-YFP-bGH | CCR5 | SFFV |
| MKC121 | | SP-FKBP-TM-EPOR-IC-2A-YFP-bGH | CCR5 | SFFV |
| MKC125 | | FKBP-TM-tEPOR-IC-2A-YFP-bGH | HBAI | HBAI |
| MKC126 | | IL6-FKBP-TM-EGFR-IC-2A-YFP-bGH | CCR5 | SFFV |
| MKC131 | | IL6-FKBP-TM-tEPOR-IC-2A-YFP-bGH | CCR5 | SFFV |
| MKC132 | | IL6-FKBP-TM-TPOR-IC-2A-YFP-bGH | CCR5 | SFFV |
| MKC133 | | IL6-FKBP-TM-SCF-IC-2A-YFP-bGH | CCR5 | SFFV |
| MKC135 | | IL6-FKBP-TM-tEPOR-IC-2A-YFP-bGH | HBAI | HBAI |
| MKC144 | | HBB-pA-IRES-IL6-FKBP-TM-tEPOR-IC | HBAI | HBAI |
| MKC145 | | IL6-FKBP-TM-div.tEPOR-IC-bGH | EPOR | EPOR |
| MCK158 | | IL6-FKBP-TM-tEPOR-IC-2A-YFP | CCR5 | PGK |

TABLE 2
Pathways upregulated in Mock d 14 vs. d 0 + EPO

| WT d 14/d 0 | | | | | |
|---|---|---|---|---|---|
| top 1% (196 | | | | | |
| genes) | | | FDR | Enrichment | |
| GO term | Description | P-value | q-value | (N, B, n, b) | |
| GO: 0006779 | porphyrin-containing compound biosynthetic process | 3.30E−12 | 5.07E−08 | 33.11 | (17887, 26, 187, 9) |
| GO: 0033014 | tetrapyrrole biosynthetic process | 3.30E−12 | 2.53E−08 | 33.11 | (17887, 26, 187, 9) |
| GO: 0006778 | porphyrin-containing compound metabolic process | 4.49E−12 | 2.30E−08 | 25.17 | (17887, 38, 187, 10) |
| GO: 0033013 | tetrapyrrole metabolic process | 2.20E−11 | 8.44E−08 | 17.83 | (17887, 59, 187, 11) |
| GO: 0046501 | protoporphyrinogen IX metabolic process | 5.33E−10 | 1.64E−06 | 52.17 | (17887, 11, 187, 6) |
| GO: 0042168 | heme metabolic process | 5.90E−10 | 1.51E−06 | 25.51 | (17887, 30, 187, 8) |
| GO: 0006783 | heme biosynthetic process | 1.25E−09 | 2.75E−06 | 31.88 | (17887, 21, 187, 7) |
| GO: 0006782 | protoporphyrinogen IX biosynthetic process | 1.44E−08 | 2.77E−05 | 53.14 | (17887, 9, 187, 5) |
| GO: 0051186 | cofactor metabolic process | 3.68E−08 | 6.29E−05 | 4.16 | (17887, 483, 187, 21) |
| GO: 0030218 | erythrocyte differentiation | 6.24E−08 | 9.59E−05 | 14.72 | (17887, 52, 187, 8) |
| GO: 0051188 | cofactor biosynthetic process | 6.52E−08 | 9.11E−05 | 6.20 | (17887, 216, 187, 14) |
| GO: 0042440 | pigment metabolic process | 1.73E−07 | 2.21E−04 | 12.97 | (17887, 59, 187, 8) |
| GO: 0046148 | pigment biosynthetic process | 3.37E−07 | 3.99E−04 | 15.22 | (17887, 44, 187, 7) |
| GO: 1901566 | organonitrogen compound biosynthetic process | 7.44E−07 | 8.17E−04 | 2.87 | (17887, 899, 187, 27) |
| GO: 0034728 | nucleosome organization | 2.49E−06 | 2.55E−03 | 5.98 | (17887, 176, 187, 11) |
| GO: 0051597 | response to methylmercury | 4.46E−06 | 4.29E−03 | 71.74 | (17887, 4, 187, 3) |
| GO: 0019755 | one-carbon compound transport | 5.36E−06 | 4.85E−03 | 31.88 | (17887, 12, 187, 4) |
| GO: 0015670 | carbon dioxide transport | 1.11E−05 | 9.46E−03 | 57.39 | (17887, 5, 187, 3) |
| GO: 0071824 | protein-DNA complex subunit organization | 1.21E−05 | 9.79E−03 | 4.63 | (17887, 248, 187, 12) |
| GO: 1901564 | organonitrogen compound metabolic process | 1.23E−05 | 9.46E−03 | 1.52 | (17887, 5041, 187, 80) |
| GO: 0045637 | regulation of myeloid cell differentiation | 1.31E−05 | 9.60E−03 | 4.59 | (17887, 250, 187, 12) |

TABLE 3
Pathways upregulated in HBA1-iEPOR + BB vs. Mock d 0

| HBA1-integrated | | | | |
|---|---|---|---|---|
| iEPOR d 14/d 0 | | | | |
| top 1% (196 | | | | |
| genes) | | | FDR | Enrichment |
| GO term | Description | P-value | q-value | (N, B, n, b) |
| GO: 0006778 | porphyrin-containing compound metabolic | 4.26E−12 | 6.55E−08 | 25.31 |
| | process | | | (17887, 38, 186, 10) |
| GO: 0033013 | tetrapyrrole metabolic process | 2.07E−11 | 1.59E−07 | 17.93 |
| | | | | (17887, 59, 186, 11) |
| GO: 0006779 | porphyrin-containing compound biosynthetic | 1.57E−10 | 8.02E−07 | 29.59 |
| | process | | | (17887, 26, 186, 8) |
| GO: 0033014 | tetrapyrrole biosynthetic process | 1.57E−10 | 6.02E−07 | 29.59 |
| | | | | (17887, 26, 186, 8) |
| GO: 0042168 | heme metabolic process | 5.66E−10 | 1.74E−06 | 25.64 |
| | | | | (17887, 30, 186, 8) |
| GO: 0051186 | cofactor metabolic process | 3.35E−08 | 8.58E−05 | 4.18 |
| | | | | (17887, 483, 186, 21) |
| GO: 0046501 | protoporphyrinogen IX metabolic process | 5.06E−08 | 1.11E−04 | 43.71 |
| | | | | (17887, 11, 186, 5) |
| GO: 0006783 | heme biosynthetic process | 5.56E−08 | 1.07E−04 | 27.48 |
| | | | | (17887, 21, 186, 6) |
| GO: 0030218 | erythrocyte differentiation | 5.99E−08 | 1.02E−04 | 14.79 |
| | | | | (17887, 52, 186, 8) |
| GO: 0042440 | pigment metabolic process | 1.66E−07 | 2.55E−04 | 13.04 |
| | | | | (17887, 59, 186, 8) |
| GO: 0051188 | cofactor biosynthetic process | 4.30E−07 | 6.02E−04 | 5.79 |
| | | | | (17887, 216, 186, 13) |
| GO: 0006782 | protoporphyrinogen IX biosynthetic process | 1.37E−06 | 1.75E−03 | 42.74 |
| | | | | (17887, 9, 186, 4) |
| GO: 0046148 | pigment biosynthetic process | 5.93E−06 | 7.01E−03 | 13.11 |
| | | | | (17887, 44, 186, 6) |
| GO: 0055076 | transition metal ion homeostasis | 5.95E−06 | 6.53E−03 | 6.98 |
| | | | | (17887, 124, 186, 9) |

TABLE 4
Pathways upregulated in EPOR-iEPOR + BB vs. Mock d 0

| EPOR-integrated | | | | | |
|---|---|---|---|---|---|
| iEPOR d 14/d 0 | | | | | |
| top 1% (196 | | | | | |
| genes) | | | FDR | Enrichment | |
| GO term | Description | P-value | q-value | (N, B, n, b) | |
| GO: 0006778 | porphyrin-containing compound metabolic process | 1.14E−13 | 1.76E−09 | 27.69 | (17887, 38, 187, 11) |
| GO: 0033013 | tetrapyrrole metabolic process | 8.72E−13 | 6.70E−09 | 19.45 | (17887, 59, 187, 12) |
| GO: 0006779 | porphyrin-containing compound biosynthetic process | 3.30E−12 | 1.69E−08 | 33.11 | (17887, 26, 187, 9) |
| GO: 0033014 | tetrapyrrole biosynthetic process | 3.30E−12 | 1.27E−08 | 33.11 | (17887, 26, 187, 9) |
| GO: 0015669 | gas transport | 8.41E−12 | 2.59E−08 | 40.27 | (17887, 19, 187, 8) |
| GO: 0042168 | heme metabolic process | 1.46E−11 | 3.73E−08 | 28.70 | (17887, 30, 187, 9) |
| GO: 0051186 | cofactor metabolic process | 4.33E−11 | 9.50E−08 | 4.95 | (17887, 483, 187, 25) |
| GO: 0046501 | protoporphyrinogen IX metabolic process | 5.33E−10 | 1.02E−06 | 52.17 | (17887, 11, 187, 6) |
| GO: 0006783 | heme biosynthetic process | 1.25E−09 | 2.14E−06 | 31.88 | (17887, 21, 187, 7) |
| GO: 0042440 | pigment metabolic process | 9.85E−09 | 1.52E−05 | 14.59 | (17887, 59, 187, 9) |
| GO: 0006782 | protoporphyrinogen IX biosynthetic process | 1.44E−08 | 2.02E−05 | 53.14 | (17887, 9, 187, 5) |
| GO: 0030218 | erythrocyte differentiation | 6.24E−08 | 7.99E−05 | 14.72 | (17887, 52, 187, 8) |
| GO: 0072593 | reactive oxygen species metabolic process | 1.64E−07 | 1.94E−04 | 9.02 | (17887, 106, 187, 10) |
| GO: 0015671 | oxygen transport | 2.20E−07 | 2.41E−04 | 34.16 | (17887, 14, 187, 5) |
| GO: 0098869 | cellular oxidant detoxification | 2.31E−07 | 2.36E−04 | 10.25 | (17887, 84, 187, 9) |
| GO: 0098754 | detoxification | 2.76E−07 | 2.65E−04 | 8.54 | (17887, 112, 187, 10) |
| GO: 0015701 | bicarbonate transport | 2.86E−07 | 2.59E−04 | 15.57 | (17887, 43, 187, 7) |
| GO: 0042744 | hydrogen peroxide catabolic process | 2.97E−07 | 2.54E−04 | 21.26 | (17887, 27, 187, 6) |
| GO: 0051187 | cofactor catabolic process | 3.30E−07 | 2.67E−04 | 11.96 | (17887, 64, 187, 8) |
| GO: 0046148 | pigment biosynthetic process | 3.37E−07 | 2.59E−04 | 15.22 | (17887, 44, 187, 7) |
| GO: 0051188 | cofactor biosynthetic process | 4.58E−07 | 3.35E−04 | 5.76 | (17887, 216, 187, 13) |
| GO: 1990748 | cellular detoxification | 6.67E−07 | 4.66E−04 | 9.06 | (17887, 95, 187, 9) |
| GO: 0036293 | response to decreased oxygen levels | 9.06E−07 | 6.06E−04 | 5.00 | (17887, 268, 187, 14) |
| GO: 0055072 | iron ion homeostasis | 1.40E−06 | 8.95E−04 | 9.94 | (17887, 77, 187, 8) |
| GO: 1901566 | organonitrogen compound biosynthetic process | 2.45E−06 | 1.51E−03 | 2.77 | (17887, 899, 187, 26) |
| GO: 0070482 | response to oxygen levels | 2.59E−06 | 1.53E−03 | 4.57 | (17887, 293, 187, 14) |
| GO: 0044237 | cellular metabolic process | 2.92E−06 | 1.66E−03 | 1.41 | (17887, 7166, 187, 106) |
| GO: 0042743 | hydrogen peroxide metabolic process | 2.95E−06 | 1.62E−03 | 14.72 | (17887, 39, 187, 6) |
| GO: 0001666 | response to hypoxia | 3.07E−06 | 1.63E−03 | 4.86 | (17887, 256, 187, 13) |
| GO: 0036294 | cellular response to decreased oxygen levels | 4.13E−06 | 2.12E−03 | 7.30 | (17887, 118, 187, 9) |
| GO: 0051597 | response to methylmercury | 4.46E−06 | 2.21E−03 | 71.74 | (17887, 4, 187, 3) |
| GO: 0019755 | one-carbon compound transport | 5.36E−06 | 2.58E−03 | 31.88 | (17887, 12, 187, 4) |
| GO: 0055076 | transition metal ion homeostasis | 6.21E−06 | 2.89E−03 | 6.94 | (17887, 124, 187, 9) |
| GO: 0008152 | metabolic process | 9.52E−06 | 4.30E−03 | 1.36 | (17887, 7797, 187, 111) |
| GO: 0015670 | carbon dioxide transport | 1.11E−05 | 4.86E−03 | 57.39 | (17887, 5, 187, 3) |
| GO: 0044248 | cellular catabolic process | 1.21E−05 | 5.18E−03 | 2.09 | (17887, 1695, 187, 37) |
| GO: 0071453 | cellular response to oxygen levels | 1.24E−05 | 5.15E−03 | 6.38 | (17887, 135, 187, 9) |
| GO: 0015893 | drug transport | 1.36E−05 | 5.50E−03 | 5.56 | (17887, 172, 187, 10) |
| GO: 0031667 | response to nutrient levels | 1.62E−05 | 6.37E−03 | 3.48 | (17887, 440, 187, 16) |
| GO: 0006979 | response to oxidative stress | 1.67E−05 | 6.42E−03 | 3.88 | (17887, 345, 187, 14) |
| GO: 0071456 | cellular response to hypoxia | 2.03E−05 | 7.60E−03 | 6.96 | (17887, 110, 187, 8) |
| GO: 0017001 | antibiotic catabolic process | 2.05E−05 | 7.51E−03 | 10.63 | (17887, 54, 187, 6) |
| GO: 0048821 | erythrocyte development | 2.48E−05 | 8.85E−03 | 22.51 | (17887, 17, 187, 4) |
| GO: 0009991 | response to extracellular stimulus | 2.78E−05 | 9.70E−03 | 3.33 | (17887, 460, 187, 16) |

TABLE 5
Pathways upregulated in PGK-iEPOR + BB vs. Mock d 0

| PGK-expressed | | | | | |
|---|---|---|---|---|---|
| iEPOR d 14/d 0 | | | | | |
| top 1% (196 | | | | | |
| genes) | | | FDR | Enrichment | |
| GO term | Description | P-value | q-value | (N, B, n, b) | |
| GO: 0006778 | porphyrin-containing compound metabolic process | 1.47E−10 | 2.26E−06 | 22.78 | (17887, 38, 186, 9) |
| GO: 0033013 | tetrapyrrole metabolic process | 4.69E−10 | 3.60E−06 | 16.30 | (17887, 59, 186, 10) |
| GO: 0006779 | porphyrin-containing compound biosynthetic process | 6.54E−09 | 3.35E−05 | 25.89 | (17887, 26, 186, 7) |
| GO: 0033014 | tetrapyrrole biosynthetic process | 6.54E−09 | 2.51E−05 | 25.89 | (17887, 26, 186, 7) |
| GO: 0042168 | heme metabolic process | 1.95E−08 | 6.00E−05 | 22.44 | (17887, 30, 186, 7) |
| GO: 0030218 | erythrocyte differentiation | 5.99E−08 | 1.53E−04 | 14.79 | (17887, 52, 186, 8) |
| GO: 0051186 | cofactor metabolic process | 1.60E−07 | 3.51E−04 | 3.98 | (17887, 483, 186, 20) |
| GO: 0042440 | pigment metabolic process | 1.66E−07 | 3.19E−04 | 13.04 | (17887, 59, 186, 8) |
| GO: 1901566 | organonitrogen compound biosynthetic process | 6.68E−07 | 1.14E−03 | 2.89 | (17887, 899, 186, 27) |
| GO: 0006783 | heme biosynthetic process | 2.05E−06 | 3.15E−03 | 22.90 | (17887, 21, 186, 5) |
| GO: 0046501 | protoporphyrinogen IX metabolic process | 3.53E−06 | 4.93E−03 | 34.97 | (17887, 11, 186, 4) |
| GO: 0044237 | cellular metabolic process | 4.19E−06 | 5.36E−03 | 1.41 | (17887, 7166, 186, 105) |
| GO: 0046148 | pigment biosynthetic process | 5.93E−06 | 7.01E−03 | 13.11 | (17887, 44, 186, 6) |

### 9. Exemplary Embodiments

Exemplary embodiments provided in accordance with the presently disclosed subject matter include, but are not limited to, the claims and the following embodiments:

Embodiment 1. A chimeric transmembrane receptor polypeptide comprising: an extramembrane dimerizer domain, wherein the extramembrane dimerizer domain induces dimerization of the chimeric transmembrane receptor polypeptide upon recognition of a dimerization signal, a transmembrane domain; and an intramembrane domain, wherein the intramembrane domain is configured to induce activation of one or more intramembrane signal pathways upon dimerization of the chimeric transmembrane receptor polypeptide in a modified primary human cell comprising the chimeric transmembrane receptor polypeptide, and wherein the one or more intramembrane signaling pathways promote survival, proliferation, and/or differentiation of the modified primary human cell.

Embodiment 2. The chimeric transmembrane receptor polypeptide of embodiment 1, wherein the extramembrane dimerizer domain comprises an FKBP domain, an mFRB domain, an HSV-TK dimerization domain, a rapamycin-inducible dimerization domain, a rapalogue-inducible dimerization domain, or a combination thereof.

Embodiment 3. The chimeric transmembrane receptor polypeptide of embodiment I or 2, wherein the dimerization signal is a pharmaceutically acceptable small molecule dimerization signal.

Embodiment 4. The chimeric transmembrane receptor polypeptide of embodiment 3, wherein the extramembrane dimerizer domain comprises an FKBP domain and wherein the small molecule dimerization signal comprises AP20187.

Embodiment 5. The chimeric transmembrane receptor polypeptide of any one of embodiments 1 to 4, wherein the intramembrane domain comprises an EPOR intracellular domain, a c-KIT/stem cell factor (SCF) receptor intracellular domain, a thrombopoietin receptor (TPOR) intracellular domain, an epidermal growth factor (EGFR) intracellular domain, an RET intracellular domain, a CSF1R intracellular domain, an IGF1R intracellular domain, or a combination thereof.

Embodiment 6. The chimeric transmembrane receptor polypeptide of any one of embodiments 1 to 5, wherein the intramembrane domain comprises an EPOR intracellular domain and wherein dimerization of the chimeric transmembrane receptor polypeptide induces activation of intramembrane signal pathways resulting in increased survival, increased proliferation, and/or increased erythroid differentiation of the modified primary human cell upon recognition of the dimerization signal.

Embodiment 7. The chimeric transmembrane receptor polypeptide of any one of embodiments 1 to 5, wherein the intramembrane domain comprises a TPOR intracellular domain and wherein dimerization of the chimeric transmembrane receptor polypeptide induces activation of intramembrane signal pathways resulting in increased survival, increased proliferation, and/or increased erythroid differentiation of the modified primary human cell upon recognition of the dimerization signal.

Embodiment 8. The chimeric transmembrane receptor polypeptide of any one of embodiments 1 to 7, wherein the intramembrane domain comprises an SCF intracellular domain and wherein dimerization of the chimeric transmembrane receptor polypeptide induces activation of intramembrane signal pathways resulting in increased survival, increased proliferation, and/or increased erythroid differentiation of the modified primary human cell upon recognition of the dimerization signal.

Embodiment 9. The chimeric transmembrane receptor polypeptide of any one of embodiments 1 to 8, wherein the extramembrane dimerizer domain is immediately adjacent to the transmembrane domain.

Embodiment 10. The chimeric transmembrane receptor polypeptide of any one of embodiments 1 to 9, wherein the chimeric transmembrane receptor polypeptide further comprises a signal peptide.

Embodiment 11. The chimeric transmembrane receptor polypeptide of embodiment 10, wherein the signal peptide promotes membrane localization of the chimeric transmembrane receptor polypeptide.

Embodiment 12. The chimeric transmembrane receptor polypeptide of embodiment 10 or 11, wherein the signal peptide comprises an IL6 signal peptide, an EPOR signal peptide, a lysozyme C signal peptide, an angiotensinogen signal peptide, an RNASE1 signal peptide, an RNASE3 signal peptide, or a modified human albumin signal peptide.

Embodiment 13. The chimeric transmembrane receptor polypeptide of any one of embodiments 1 to 12, wherein the chimeric transmembrane receptor polypeptide further comprises a linker peptide.

Embodiment 14. The chimeric transmembrane receptor polypeptide of embodiment 13, wherein the linker peptide comprises the amino acid sequence GGGGS.

Embodiment 15. The chimeric transmembrane receptor polypeptide of any one of embodiments 1 to 14, wherein the chimeric transmembrane receptor polypeptide comprises an amino acid sequence having at least 80% identity to any one of SEQ ID NOS: 1 to 3 and 7 to 11.

Embodiment 16. A recombinant nucleic acid encoding the chimeric transmembrane receptor polypeptide of any one of embodiments 1 to 15.

Embodiment 17. A DNA construct comprising a promoter operably linked to the recombinant nucleic acid of embodiment 16.

Embodiment 18. The DNA construct of embodiment 17, wherein the promoter is an endogenous EPOR promoter, an endogenous HBA 1 promoter, an endogenous TPOR promoter, a constitutive SFFV promoter, a constitutive PGK promoter, or a constitutive UbC promoter.

Embodiment 19. A vector comprising the recombinant nucleic acid of embodiment 16 or the DNA construct of embodiment 17 or 18.

Embodiment 20. A host cell comprising the recombinant nucleic acid of embodiment 16, the DNA construct of embodiment 17 or 18, or the vector of embodiment 19.

Embodiment 21. The host cell of embodiment 20, wherein the recombinant nucleic acid, DNA construct, or vector is integrated into the CCR5 locus, the HBA 1 locus, or the EPOR locus.

Embodiment 22. The host cell of embodiment 20, wherein the host cell is a eukaryotic cell.

Embodiment 23. The host cell of embodiment 20 or 21, wherein the host cell is a primary human cell.

Embodiment 24. The host cell of any one of embodiments 20 to 23, wherein the host cell is a hematopoietic stem cell, a hematopoietic progenitor cell, a T cell, a B cell, an airway basal stem cell, or a pluripotent stem cell (PSC).

Embodiment 25. The host cell of any one of embodiments 20 to 24, wherein the host cell is a hematopoietic stem and progenitor cell (HSPC).

Embodiment 26. The host cell of any one of embodiments 20 to 25, wherein the host cell was derived from a patient who is a carrier of an allele that causes a genetic disorder.

Embodiment 27. The host cell of embodiment 26, wherein the genetic disorder is beta-thalassemia, sickle cell disease (SCD), severe combined immunodeficiency (SCID), mucopolysaccharidosis type 1, Cystic Fibrosis, Gaucher disease, Krabbe disease, X-linked chronic granulomatous disease (X-CGD), or a combination thereof.

Embodiment 28. The host cell of embodiment 26 or 27, wherein the genetic disorder is a hemoglobinopathy.

Embodiment 29. The host cell of embodiment 28, wherein the hemoglobinopathy is beta-thalassemia or sickle cell disease.

Embodiment 30. The host cell of any one of embodiments 25 to 29, wherein the genome of the host cell is edited to alter the allele associated with the genetic disorder.

Embodiment 31. A method of inducing erythroid differentiation of an HSPC, the method comprising contacting the HSPC of embodiment 25 with the dimerization signal.

Embodiment 32. A method of tuning red blood cell levels in a patient, the method comprising:
(i) editing an HSPC so that it can express at least one of the chimeric transmembrane receptor polypeptides of any one of embodiments 1 to 15;
(ii) transferring the resulting HSPC to the patient;
(iii)administering a first quantity of the dimerization signal to the patient;
(iv) monitoring red blood cell levels in the patient; and
(v) administering a second quantity of the dimerization signal to the patient that is the same, less, or more than the first quantity of dimerization signal.

Embodiment 33. A method of increasing the proportion of red blood cells with an altered version of an allele associated with a genetic disorder, the method comprising:
(i) creating an edited HSPC by introducing an altered version of the allele associated with a genetic disorder and a polynucleotide that encodes at least one of the chimeric transmembrane receptor polypeptides of any one of embodiments 1 to 15 into an HSPC;
(ii) transferring the edited HSPC to the patient; and
(iii) administering the dimerization signal to the patient.

Embodiment 34. The method of embodiment 32 or 33, wherein the HSPC is derived from the patient's own cells.

Embodiment 35. The method of embodiment 32 or 33, wherein the HSPC is derived from an allogeneic donor's cells.

Embodiment 36. The method of embodiment 35, wherein the genetic disorder is beta-thalassemia, sickle cell disease (SCD), severe combined immunodeficiency (SCID), mucopolysaccharidosis type 1, Cystic Fibrosis, Gaucher disease, Krabbe disease. X-linked chronic granulomatous disease (X-CGD), or a combination thereof.

Embodiment 37. A method of genetically modifying a primary human cell, the method comprising introducing into the cell a chimeric transmembrane receptor polypeptide of any one of embodiments I to 15.

Embodiment 38. The method of embodiment 37, the method further comprising:
(i)introducing into the cell a site-directed nuclease (SDN) targeted to a cleavage site at a genetic locus of interest; and
(ii) introducing a homologous repair template into the cell, wherein the homologous repair template comprises a nucleotide sequence that is homologous to the locus of interest,
wherein the site-directed nuclease cleaves the locus at the cleavage site, and the homologous repair template is integrated at the site of the cleaved locus by homology directed repair (HDR).

Embodiment 39. The method of embodiment 38, wherein the SDN is an RNA-guided nuclease and the method further comprises introducing into the cell a single guide RNA (sgRNA) targeting the cleavage site, wherein the sgRNA directs the RNA-guided nuclease to the cleavage site.

Embodiment 40. The method of embodiment 39, wherein the sgRNA comprises 2′-O-methyl-3′-phosphorothioate (MS) modifications at one or more nucleotides.

Embodiment 41. The method of embodiment 40, wherein the MS modifications are present at the terminal nucleotides of the 5′ and 3′ ends.

Embodiment 42. The method of anyone of embodiments 39 to 41, wherein the RNA-guided nuclease is Cas9.

Embodiment 43. The method of any one of embodiments 39 to 42, wherein the sgRNA and RNA-guided nuclease are introduced into the cell as a ribonucleoprotein (RNP).

Embodiment 44. The method of embodiment 43, wherein the RNP is introduced into the cell by electroporation.

Embodiment 45. The method of any one of embodiments 38 to 44, wherein the homologous repair template is introduced into the cell using an adeno-associated virus serotype 6 (AAV6) vector.

Embodiment 46. The method of any one of embodiments 38 to 45, wherein the primary human cell is a hematopoietic stem cell, a hematopoietic progenitor cell, a T cell, a B cell, an airway basal stem cell, or a pluripotent stem cell (PSC).

Embodiment 47. The method of any one of embodiments 38 to 46, wherein the locus of interest is a gene selected from the group consisting of Erythropoietin Receptor (EPOR), Hemoglobin Subunit Beta (HBB), C-C Motif Chemokine Receptor 5 (CCR5), Interleukin 2 Receptor Subunit Gamma (IL2RG), Hemoglobin Subunit Alpha 1 (HBA1), Stimulator Of Interferon Response cGAMP Interactor 1 (STING1) and Cystic Fibrosis Transmembrane Conductance Regulator (CFTR).

Embodiment 48. A method of treating a genetic disorder in a human subject in need thereof, the method comprising:
(i) providing an isolated primary cell from the subject;
(ii) genetically modifying the primary cell using the method of any one of embodiments 37 to 47, wherein the integration of the homologous donor template at the locus of interest in the cell alters an allele at the locus that is associated with the genetic disorder or leads to the expression of a therapeutic protein in the cell that is absent or deficient in the subject; and
(iii) reintroducing the genetically modified cell into the subject.

Embodiment 49. The method of embodiment 48, wherein the genetic disorder is beta-thalassemia, sickle cell disease (SCD), severe combined immunodeficiency (SCID), mucopolysaccharidosis type 1, Cystic Fibrosis, Gaucher disease, Krabbe disease, X-linked chronic granulomatous disease (X-CGD), or a combination thereof.

Embodiment 50. A method of generating a population of red blood cell in vitro, the method comprising:
(i) editing one or more HSPCs to express at least one of the chimeric transmembrane receptor polypeptides of any one of embodiments I to 15; and
(ii) contacting the one or more HSPCs with a dimerization signal.

Embodiment 51. The method of embodiment 50, wherein the dimerization signal is a small molecule dimerization signal.

Embodiment 52. The method of embodiment 51, wherein the small molecule dimerization signal is AP20187 (BB dimerizer).

Embodiment 53. The method of any one of embodiments 50 to 52, wherein step (ii) further comprises contacting the one or more HSPCs with erythropoietin (EPO).

Embodiment 54. The method of any one of embodiments 50 to 53, wherein the method is conducted in a bioreactor.

What is claimed is:

1. A chimeric transmembrane receptor polypeptide comprising:
an extramembrane dimerizer domain, wherein the extramembrane dimerizer domain induces dimerization of the chimeric transmembrane receptor polypeptide upon recognition of a dimerization signal;
a transmembrane domain; and
an intramembrane domain, wherein the intramembrane domain is configured to induce activation of one or more intramembrane signal pathways upon dimerization of the chimeric transmembrane receptor polypeptide in a modified primary human cell comprising the chimeric transmembrane receptor polypeptide, and wherein the one or more intramembrane signaling pathways promote survival, proliferation, and/or differentiation of the modified primary human cell.

2. The chimeric transmembrane receptor polypeptide of claim 1, wherein the extramembrane dimerizer domain comprises an FKBP domain, an mFRB domain, an HSV-TK dimerization domain, a rapamycin-inducible dimerization domain, a rapalogue-inducible dimerization domain, or a combination thereof.

3. The chimeric transmembrane receptor polypeptide of claim 1 or 2, wherein the dimerization signal is a pharmaceutically acceptable small molecule dimerization signal.

4. The chimeric transmembrane receptor polypeptide of claim 3, wherein the extramembrane dimerizer domain comprises an FKBP domain and wherein the small molecule dimerization signal comprises AP20187.

5. The chimeric transmembrane receptor polypeptide of claim 1, wherein the intramembrane domain comprises an EPOR intracellular domain, a c-KIT/stem cell factor (SCF) receptor intracellular domain, a thrombopoietin receptor (TPOR) intracellular domain, an epidermal growth factor (EGFR) intracellular domain, an RET intracellular domain, a CSF1R intracellular domain, an IGF1R intracellular domain, or a combination thereof.

6. The chimeric transmembrane receptor polypeptide of claim 1, wherein the intramembrane domain comprises an EPOR intracellular domain and wherein dimerization of the chimeric transmembrane receptor polypeptide induces activation of intramembrane signal pathways resulting in increased survival, increased proliferation, and/or increased erythroid differentiation of the modified primary human cell upon recognition of the dimerization signal.

7. The chimeric transmembrane receptor polypeptide of claim 1, wherein the intramembrane domain comprises a TPOR intracellular domain and wherein dimerization of the chimeric transmembrane receptor polypeptide induces activation of intramembrane signal pathways resulting in increased survival, increased proliferation, and/or increased erythroid differentiation of the modified primary human cell upon recognition of the dimerization signal.

8. The chimeric transmembrane receptor polypeptide of claim 1, wherein the intramembrane domain comprises an SCF intracellular domain and wherein dimerization of the chimeric transmembrane receptor polypeptide induces activation of intramembrane signal pathways resulting in increased survival, increased proliferation, and/or increased erythroid differentiation of the modified primary human cell upon recognition of the dimerization signal.

9. The chimeric transmembrane receptor polypeptide of claim 1, wherein the extramembrane dimerizer domain is immediately adjacent to the transmembrane domain.

10. The chimeric transmembrane receptor polypeptide of claim 1, wherein the chimeric transmembrane receptor polypeptide further comprises a signal peptide.

11. The chimeric transmembrane receptor polypeptide of claim 10, wherein the signal peptide promotes membrane localization of the chimeric transmembrane receptor polypeptide.

12. The chimeric transmembrane receptor polypeptide of claim 10 or 11, wherein the signal peptide comprises an 1L6 signal peptide, an EPOR signal peptide, a lysozyme C signal peptide, an angiotensinogen signal peptide, an RNASE1 signal peptide, an RNASE3 signal peptide, or a modified human albumin signal peptide.

13. The chimeric transmembrane receptor polypeptide of claim 1, wherein the chimeric transmembrane receptor polypeptide further comprises a linker peptide.

14. The chimeric transmembrane receptor polypeptide of claim 13, wherein the linker peptide comprises the amino acid sequence GGGGS.

15. The chimeric transmembrane receptor polypeptide of claim 1, wherein the chimeric transmembrane receptor polypeptide comprises an amino acid sequence having at least 80% identity to any one of SEQ ID NOS: 1 to 3 and 7 to 11.

16. A recombinant nucleic acid encoding the chimeric transmembrane receptor polypeptide of claim 1.

17. A DNA construct comprising a promoter operably linked to the recombinant nucleic acid of claim 16.

18. The DNA construct of claim 17, wherein the promoter is an endogenous EPOR promoter, an endogenous HBA1 promoter, an endogenous TPOR promoter, a constitutive SFFV promoter, a constitutive PGK promoter, or a constitutive UbC promoter.

19. A vector comprising the recombinant nucleic acid of claim 16 or the DNA construct of claim 17.

20. A host cell comprising the recombinant nucleic acid of claim 16, the DNA construct of claim 17, or the vector of claim 19.

21. The host cell of claim 20, wherein the recombinant nucleic acid, DNA construct, or vector is integrated into the CCR5 locus, the HBA1 locus, or the EPOR locus.

22. The host cell of claim 20, wherein the host cell is a eukaryotic cell.

23. The host cell of claim 20 or 21, wherein the host cell is a primary human cell.

24. The host cell of claim 20, wherein the host cell is a hematopoietic stem cell, a hematopoietic progenitor cell, a T cell, a B cell, an airway basal stem cell, or a pluripotent stem cell (PSC).

25. The host cell of claim 20, wherein the host cell is a hematopoietic stem and progenitor cell (HSPC).

26. The host cell of claim 20, wherein the host cell was derived from a patient who is a carrier of an allele that causes a genetic disorder.

27. The host cell of claim 26, wherein the genetic disorder is beta-thalassemia, sickle cell disease (SCD), severe combined immunodeficiency (SCID), mucopolysaccharidosis type 1, Cystic Fibrosis, Gaucher disease, Krabbe disease. X-linked chronic granulomatous disease (X-CGD), or a combination thereof.

28. The host cell of claim 26 or 27, wherein the genetic disorder is a hemoglobinopathy.

29. The host cell of claim 28, wherein the hemoglobinopathy is beta-thalassemia or sickle cell disease.

30. The host cell of claim 25, wherein the genome of the host cell is edited to alter the allele associated with the genetic disorder.

31. A method of inducing erythroid differentiation of an HSPC, the method comprising contacting the HSPC of claim 25 with the dimerization signal.

32. A method of tuning red blood cell levels in a patient, the method comprising:
(i) editing an HSPC so that it can express at least one of the chimeric transmembrane receptor polypeptides of claim 1;
(ii) transferring the resulting HSPC to the patient;
(iii) administering a first quantity of the dimerization signal to the patient;
(iv) monitoring red blood cell levels in the patient; and
(v) administering a second quantity of the dimerization signal to the patient that is the same, less, or more than the first quantity of dimerization signal.

33. A method of increasing the proportion of red blood cells with an altered version of an allele associated with a genetic disorder, the method comprising:
(i) creating an edited HSPC by introducing an altered version of the allele associated with a genetic disorder and a polynucleotide that encodes at least one of the chimeric transmembrane receptor polypeptides of claim 1 into an HSPC;
(ii) transferring the edited HSPC to the patient; and
(iii) administering the dimerization signal to the patient.

34. The method of claim 32 or 33, wherein the HSPC is derived from the patient's own cells.

35. The method of claim 32 or 33, wherein the HSPC is derived from an allogeneic donor's cells.

36. The method of claim 35, wherein the genetic disorder is beta-thalassemia, sickle cell disease (SCD), severe combined immunodeficiency (SCID), mucopolysaccharidosis type 1, Cystic Fibrosis, Gaucher disease, Krabbe disease. X-linked chronic granulomatous disease (X-CGD), or a combination thereof.

37. A method of genetically modifying a primary human cell, the method comprising introducing into the cell a chimeric transmembrane receptor polypeptide of claim 1.

38. The method of claim 37, the method further comprising:
(i) introducing into the cell a site-directed nuclease (SDN) targeted to a cleavage site at a genetic locus of interest; and
(ii) introducing a homologous repair template into the cell, wherein the homologous repair template comprises a nucleotide sequence that is homologous to the locus of interest,
wherein the site-directed nuclease cleaves the locus at the cleavage site, and the homologous repair template is integrated at the site of the cleaved locus by homology directed repair (HDR).

39. The method of claim 38, wherein the SDN is an RNA-guided nuclease and the method further comprises introducing into the cell a single guide RNA (sgRNA) targeting the cleavage site, wherein the sgRNA directs the RNA-guided nuclease to the cleavage site.

40. The method of claim 39, wherein the sgRNA comprises 2′-O-methyl-3′-phosphorothioate (MS) modifications at one or more nucleotides.

41. The method of claim 40, wherein the MS modifications are present at the terminal nucleotides of the 5′ and 3′ ends.

42. The method of claim 39, wherein the RNA-guided nuclease is Cas9.

43. The method of claim 39, wherein the sgRNA and RNA-guided nuclease are introduced into the cell as a ribonucleoprotein (RNP).

44. The method of claim 43, wherein the RNP is introduced into the cell by electroporation.

45. The method of claim 38, wherein the homologous repair template is introduced into the cell using an adeno-associated virus serotype 6 (AAV6) vector.

46. The method of claim 38, wherein the primary human cell is a hematopoietic stem cell, a hematopoietic progenitor cell, a T cell, a B cell, an airway basal stem cell, or a pluripotent stem cell (PSC).

47. The method of claim 38, wherein the locus of interest is a gene selected from the group consisting of Erythropoietin Receptor (EPOR), Hemoglobin Subunit Beta (HBB), C-C Motif Chemokine Receptor 5 ((CCR5), Interleukin 2 Receptor Subunit Gamma (IL2RG), Hemoglobin Subunit Alpha 1 (HBA1), Stimulator Of Interferon Response cGAMP Interactor 1 (STING1) and Cystic Fibrosis Transmembrane Conductance Regulator (CFTR).

48. A method of treating a genetic disorder in a human subject in need thereof, the method comprising:
(i) providing an isolated primary cell from the subject;
(ii) genetically modifying the primary cell using the method of claim 37, wherein the integration of the homologous donor template at the locus of interest in the cell alters an allele at the locus that is associated with the genetic disorder or leads to the expression of a therapeutic protein in the cell that is absent or deficient in the subject; and
(iii) reintroducing the genetically modified cell into the subject.

49. The method of claim 48, wherein the genetic disorder is beta-thalassemia, sickle cell disease (SCD), severe combined immunodeficiency (SCID), mucopolysaccharidosis type 1, Cystic Fibrosis, Gaucher disease, Krabbe disease, X-linked chronic granulomatous disease (X-CGD), or a combination thereof.

50. A method of generating a population of red blood cell in vitro, the method comprising:
(i) editing one or more HSPCs to express at least one of the chimeric transmembrane receptor polypeptides of claim 1; and
(ii) contacting the one or more HSPCs with a dimerization signal.

51. The method of claim 50, wherein the dimerization signal is a small molecule dimerization signal.

52. The method of claim 51, wherein the small molecule dimerization signal is AP20187 (BB dimerizer).

53. The method of claim 50, wherein step (ii) further comprises contacting the one or more HSPCs with erythropoietin (EPO).

54. The method of claim 50, wherein the method is conducted in a bioreactor.


SEQUENCE LISTING (43 sequences)

SEQ ID NO: 1 - protein, 628 aa, synthetic construct
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPGGGGSMLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 2 - protein, 374 aa, synthetic construct
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 3 - protein, 628 aa, synthetic construct
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVGGGGSMLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 4 - protein, 29 aa, synthetic construct
MNSFSTSAFGPVAFSLGLLLVLPAAFPAP

SEQ ID NO: 5 - protein, 24 aa, synthetic construct
MDHLGASLWPQVGSLCLLLAGAAW

SEQ ID NO: 6 - protein, 147 aa, synthetic construct
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 7 - protein, 408 aa, synthetic construct
MNSFSTSAFGPVAFSLGLLLVLPAAFPAPGGGGSMLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 8 - protein, 602 aa, synthetic construct
MNSFSTSAFGPVAFSLGLLLVLPAAFPAPGGGGSMLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 9 - protein, 294 aa, synthetic construct
MNSFSTSAFGPVAFSLGLLLVLPAAFPAPGGGGSMLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 10 - protein, 715 aa, synthetic construct
MNSFSTSAFGPVAFSLGLLLVLPAAFPAPGGGGSMLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 11 - protein, 338 aa, synthetic construct
MNSFSTSAFGPVAFSLGLLLVLPAAFPAPGGGGSMLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRP

SEQ ID NO: 12 - DNA, 946 bp, synthetic construct
  source 1..946: mol_type = other DNA
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccacc

SEQ ID NO: 13 - DNA, 879 bp, synthetic construct
  source 1..879: mol_type = other DNA
tggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 14 - DNA, 400 bp, synthetic construct
  source 1..400: mol_type = other DNA
aagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttc

SEQ ID NO: 15 - DNA, 400 bp, synthetic construct
  source 1..400: mol_type = other DNA
tgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaa

SEQ ID NO: 16 - DNA, 900 bp, synthetic construct
  source 1..900: mol_type = other DNA
attagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacacatatatatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacacc

SEQ ID NO: 17 - DNA, 900 bp, synthetic construct
  source 1..900: mol_type = other DNA
ggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaa

SEQ ID NO: 18 - DNA, 964 bp, synthetic construct
  source 1..964: mol_type = other DNA
gagatgggggtctcactatgtttcctaggctggtcttgaactcctgggttcaaatgatcctcccacctcagcctcccaaagtactgggattatagtgctggtgtaaaccactgcacctggccatggccaggattaaagggagaatgaccaaggtatattgaactcctatgcacccttcaataccctgttccatttacccttttgtagggccttgctgatgcttcagccaaaacccctgtcccctggccctgatgtactcctctgcctccattgtgatcacagggaccaagtgtatctgtgcctctatgactgggagtggagggggaattggtgagtattcaatgagtcatatctatgtaactatttatattggcttcaacagggctctgaagcagaagatctggcctggcatcccgagcccagagagcgagtttgaaggcctcttcaccacccacaagggtaacttccaggtaggtggcctggttgtcccctcagtgcctgggcttccctgcttcttgcagccaaactgcaggcctctctgagcaggttggtgctatttcttcagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtcca

SEQ ID NO: 19 - DNA, 962 bp, synthetic construct
  source 1..962: mol_type = other DNA
acactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttaggacaccaggctgcagatgatcagggatccaatatgactcagagaaccagtgcagactcaagacttatggaacagggatggcgaggcctctctcaggagcaggggcattgctgattttgtctgcccaatccatcctgctcaggaaaccacaaccttgcagtatttttaaatatgtatagtttttttttgtatctatatatatatatacacatatgtatgtaagtttttctaccatgatttctacaaacaccctttaagtcccatcttcccctgggcataggccatagggatagaagttaaagttcttgagcttattcagaagctggatctgcaatctgaatgctactcataacataacaaaatagtatgttaaacagctcttaaatcttactggcttaccacattaaatgatttctctctcctaactcagctcaaatgggcagccatccatgggatgagtcagaggttcagactcttccagtctgtagctctaccttctcttagggtacttagatggatcccctgttctacaaactgccagtcagcaagggaagaaaaagggcagcaatgaccctcaatgggccatttgagggatctggcctggaaatgggcttcctctcttcttctcacacctcactggctggaaacagtcacatgaccccagtcacatgaaaggccaggaaacttagtttagctgtacacccaggaagggcaaagctgtttaagggccactagctagtctctgccactaat

SEQ ID NO: 20 - DNA, 1527 bp, synthetic construct
  source 1..1527: mol_type = other DNA
atggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttag

SEQ ID NO: 21 - DNA, 1314 bp, synthetic construct
  source 1..1314: mol_type = other DNA
atggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtcca

SEQ ID NO: 22 - DNA, 912 bp, synthetic construct
  source 1..912: mol_type = other DNA
atgcttgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggaggaggaggtagtcctctgattctcaccctgagcctgattctggtcgtgattctcgtcctcctcacagtcctggccctcctgagccatcggagagccctcaaacaaaaaatttggcccggaattccctcccccgaatccgaattcgagggactgtttacaacacataaaggcaattttcaactctggctctatcaaaacgacggatgtctctggtggtccccttgtacaccttttaccgaagatccccccgccagcctcgaggtgctgagcgaacggtgttggggcaccatgcaagccgtcgaacccggcaccgacgacgaaggacctctcctcgaacccgtcggaagcgaacacgctcaagacacatacctcgtcctcgataagtggctgctccctagaaatccccctagcgaagatctgcccggccccggcggaagcgtcgatatcgtcgctatggacgagggaagcgaggccagcagctgtagcagcgccctggctagcaaaccttcccccgaaggcgctagcgccgcttccttcgaatataccattctcgatccttccagccaactgctgcggccc

SEQ ID NO: 23 - DNA, 20 bp, synthetic construct
  source 1..20: mol_type = other DNA
ggcaagaagcatggccaccg

SEQ ID NO: 24 - DNA, 20 bp, synthetic construct
  source 1..20: mol_type = other DNA
gcagcatagtgagcccagaa

SEQ ID NO: 25 - DNA, 20 bp, synthetic construct
  source 1..20: mol_type = other DNA
gggtgggaaaatagaccaat

SEQ ID NO: 26 - DNA, 20 bp, synthetic construct
  source 1..20: mol_type = other DNA
tatggttaagttcatgtcat

SEQ ID NO: 27 - DNA, 20 bp, synthetic construct
  source 1..20: mol_type = other DNA
taggaaggggataagtaaca

SEQ ID NO: 28 - DNA, 20 bp, synthetic construct
  source 1..20: mol_type = other DNA
agctcagggcacagtgtcca

SEQ ID NO: 29 - DNA, 587 bp, synthetic construct
  source 1..587: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 30 - DNA, 78 bp, synthetic construct
  source 1..78: mol_type = other DNA
cgcaagcgccgcagcggcagcggcgagggccgcggcagcctgctgacctgcggcgacgtggaggagaaccccggcccc

SEQ ID NO: 31 - DNA, 78 bp, synthetic construct
  source 1..78: mol_type = other DNA
cgcaaaagacgctccggttctggagagggcagggggagtcttcttacatgcggggatgttgaggagaatcccggaccc

SEQ ID NO: 32 - DNA, 1014 bp, synthetic construct
  source 1..1014: mol_type = other DNA
atgaactccttctccacaagcgccttcggtccagttgccttctccctggggctgctcctggtgttgcctgctgccttccctgccccaggcggcggcggaagcatgcttgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggaggaggaggtagtcccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtcca

SEQ ID NO: 33 - DNA, 2631 bp, synthetic construct
  source 1..2631: mol_type = other DNA
aagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttcattaccctgttatccctagggaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagctgtatttccaaaacctaggttaattaagcttaaggatccatcgccaccatgaactccttctccacaagcgccttcggtccagttgccttctccctggggctgctcctggtgttgcctgctgccttccctgccccaggcggcggcggaagcatgcttgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggaggaggaggtagtcccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccataactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaa

SEQ ID NO: 34 - DNA, 2842 bp, synthetic construct
  source 1..2842: mol_type = other DNA
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccaccatgaactccttctccacaagcgccttcggtccagttgccttctccctggggctgctcctggtgttgcctgctgccttccctgccccaggcggcggcggaagcatgcttgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggaggaggaggtagtcccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccataatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 35 - DNA, 3246 bp, synthetic construct
  source 1..3246: mol_type = other DNA
gagatgggggtctcactatgtttcctaggctggtcttgaactcctgggttcaaatgatcctcccacctcagcctcccaaagtactgggattatagtgctggtgtaaaccactgcacctggccatggccaggattaaagggagaatgaccaaggtatattgaactcctatgcacccttcaataccctgttccatttacccttttgtagggccttgctgatgcttcagccaaaacccctgtcccctggccctgatgtactcctctgcctccattgtgatcacagggaccaagtgtatctgtgcctctatgactgggagtggagggggaattggtgagtattcaatgagtcatatctatgtaactatttatattggcttcaacagggctctgaagcagaagatctggcctggcatcccgagcccagagagcgagtttgaaggcctcttcaccacccacaagggtaacttccaggtaggtggcctggttgtcccctcagtgcctgggcttccctgcttcttgcagccaaactgcaggcctctctgagcaggttggtgctatttcttcagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccacgcaaaagacgctccggttctggagagggcagggggagtcttcttacatgcggggatgttgaggagaatcccggacccatgaactccttctccacaagcgccttcggtccagttgccttctccctggggctgctcctggtgttgcctgctgccttccctgccccaggcggcggcggaagcatgcttgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggaggaggaggtagtcctctgattctgaccctgagcctgatcctggtggtgatcctggtgctgctgacagtgctggccctgctgagccacagaagagccctgaagcagaagatctggcctggcatcccctctcccgagagcgagttcgagggactgtttaccacacacaagggcaacttccagctgtggctgtaccagaacgacggctgcctgtggtggtccccctgcacacctttcaccgaagatcctccagcctccctcgaggtgctgagcgaacggtgctggggcaccatgcaggccgtggaacccggaaccgacgacgaaggccccctgctggaacctgtgggctccgaacacgcccaggacacctacctggtcctggataagtggctgcttccccggaatcctccaagcgaggatctgcctggccctggcggatctgtggacatcgtggccatggacgagggcagcgaggccagctcttgtagcagcgccctggcttctaaacctagcccagagggcgccagcgctgctagcttcgagtacaccatcctggaccctagcagccaactgctgagaccttaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggacactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttaggacaccaggctgcagatgatcagggatccaatatgactcagagaaccagtgcagactcaagacttatggaacagggatggcgaggcctctctcaggagcaggggcattgctgattttgtctgcccaatccatcctgctcaggaaaccacaaccttgcagtatttttaaatatgtatagtttttttttgtatctatatatatatatacacatatgtatgtaagtttttctaccatgatttctacaaacaccctttaagtcccatcttcccctgggcataggccatagggatagaagttaaagttcttgagcttattcagaagctggatctgcaatctgaatgctactcataacataacaaaatagtatgttaaacagctcttaaatcttactggcttaccacattaaatgatttctctctcctaactcagctcaaatgggcagccatccatgggatgagtcagaggttcagactcttccagtctgtagctctaccttctcttagggtacttagatggatcccctgttctacaaactgccagtcagcaagggaagaaaaagggcagcaatgaccctcaatgggccatttgagggatctggcctggaaatgggcttcctctcttcttctcacacctcactggctggaaacagtcacatgaccccagtcacatgaaaggccaggaaacttagtttagctgtacacccaggaagggcaaagctgtttaagggccactagctagtctctgccactaat

SEQ ID NO: 36 - protein, 5 aa, synthetic construct
GGGGS

SEQ ID NO: 37 - protein, 9 aa, synthetic construct
GGSGGSGGS

SEQ ID NO: 38 - protein, 6 aa, synthetic construct
GSGSGS

SEQ ID NO: 39 - protein, 12 aa, synthetic construct
ESKYGPPCPPCP

SEQ ID NO: 40 - protein, 12 aa, synthetic construct
ESKYGPPAPPAP

SEQ ID NO: 41 - DNA, 23 bp, synthetic construct
  source 1..23: mol_type = other DNA
atatgcagccactcctagagctc

SEQ ID NO: 42 - DNA, 23 bp, synthetic construct
  source 1..23: mol_type = other DNA
ctggttcagagaaatgatgggca

SEQ ID NO: 43 - DNA, 20 bp, synthetic construct
  source 1..20: mol_type = other DNA
aggaaaccggagaaagggta